(19) United States
(12) Patent Application Publication
(10) Pub. No.: US 2011/0082158 A1
(43) Pub. Date: Apr. 7, 2011

(54) SELECTIVE PROTON COUPLED FOLATE TRANSPORTER AND FOLATE RECEPTOR, AND GARFTASE AND/OR OTHER FOLATE METABOLIZING ENZYMES INHIBITOR COMPOUNDS AND METHODS OF USING THE SAME

(76) Inventors: Aleem Gangjee, Allison Park, PA (US); Larry H. Matherly, Farmington Hills, MI (US)

(21) Appl. No.: 12/902,310

(22) Filed: Oct. 12, 2010

Related U.S. Application Data
(63) Continuation-in-part of application No. 12/242,988, filed on Oct. 1, 2008.

(51) Int. Cl. A61K 31/519; C07D 487/04; C07D 491/048; A61P 35/00; A61P 37/06; C12N 5/09; C12N 9/99

(52) U.S. Cl. 514/260.1; 544/280; 514/265.1; 544/278; 435/375; 435/184

(57) ABSTRACT

Fused cyclic pyrimidine compounds, including tautomers thereof, and pharmaceutically acceptable salts, prodrugs, solvates and hydrates thereof, are disclosed having the general Formula II:

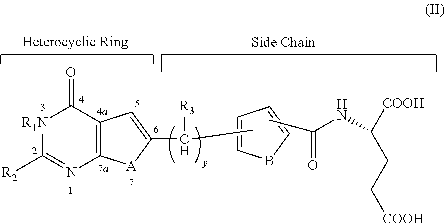

These compounds are useful in methods for treating cancer, selectively targeting cancerous cells via the proton coupled folate transporter, folate receptor alpha, and/or folate receptor beta pathways, inhibiting GARFTase and/or other folate metabolizing enzymes in cancerous cells, and selectively targeting activated macrophages in a patient having an autoimmune disease, such as rheumatoid arthritis.

### CROSS-REFERENCE TO RELATED APPLICATION

This utility patent application is a continuation-in-part patent application of U.S. patent application Ser. No. 12/242,988, filed on Oct. 1, 2008, and claims the benefit of priority thereto. The entirety of U.S. patent application Ser. No. 12/242,988 is incorporated by reference herein.

### GOVERNMENT INTEREST

This invention was supported in part by the National Institutes of Health, U.S. Department of Health and Human Services under Contract No. R01 CA125153. The Government may have certain rights in this invention.

### FIELD OF THE INVENTION

The present invention relates to selective proton coupled folate transporter (PCFT) and alpha folate receptor (FR alpha), beta folate receptor (FR beta), and glycinamide ribonucleotide formyltransferase (GARFTase) enzyme inhibitor compounds, and their methods of use. Preferably, these compounds have heterocycloalkyl-carbonyl-L-glutamate substituents or heterocycloaryl-carbonyl-L-glutamate substituents. The compounds of this invention may be made into salts that are water soluble for providing orally active selective antitumor agents.

### BACKGROUND OF THE INVENTION

Known cancer chemotherapy agents target both normal and cancerous tumor cells. This lack of selectivity for tumor cells results in cytotoxicity to the normal cells and is one of the major causes of chemotherapeutic failure in the treatment of cancer. Further, advanced stage and chemotherapeutic agent resistant tumors may be difficult to treat with know chemotherapeutic agents such as for example but not limited to carboplatin or paclitaxel (docitaxel).

Folates are members of the B Class of vitamins that are cofactors for the synthesis of nucleotide precursors. serine and methionine in one-carbon transfer reactions. Since mammals cannot synthesize folates de novo, cellular uptake of these derivatives is essential for cell growth and tissue regeneration. Reflecting their hydrophilic anionic character, folates do not cross biological membranes by diffusion alone. Accordingly, mammalian cells have evolved sophisticated membrane transport systems for facilitating accumulation of folates.

The ubiquitously expressed reduced folate carrier (RFC) is the major transport system for folates in mammalian cells and mediates concentrative uptake of folate substrates. RFC is a member of the major facilitator superfamily of transporters and is an integral transmembrane protein with micromolar affinity for its physiologic substrate, 5-methyl tetrahydrofolate. Importantly, RFC is also the primary transporter of clinically relevant antifolate drugs used for cancer including methotrexate (MTX), raltitrexed (ZD1694, Tomudex) (RTX), and pemetrexed (LY231514, Alimta) (PMX). Loss of RFC levels or function is a common mode of antifolate resistance. While a previously unrecognized proton-coupled folate transporter (PCFT) was recently reported to contribute to folate absorption in the duodenum, its tissue-specificity and overall role in folate homeostasis are not clear yet.

The family of folate receptors (FRs) represents yet another mode of folate uptake into mammalian cells. The FRs are high affinity folate binding proteins encoded by three distinct genes, designated FR alpha, FR beta and FR gamma, localized to chromosome 11q13.3-q13.5. In contrast to RFC and PCFT, FR alpha and FR beta are anchored in plasma membranes by a glycosyl phosphatidylinositol (GPI) anchor. FR gamma contains no GPI anchor and is secreted. Whereas FR alpha and FR beta (but not FR gamma) mediate cellular accumulation of folate at low (nanomolar) concentrations by receptor-mediated endocytosis, these homologous proteins show differences in binding affinities for reduced folate substrates.

The high affinity FRs offer a potential means of selective tumor targeting, given their restricted pattern of tissue expression and function. For instance, FR alpha is expressed on the apical membrane surface of normal tissues such as kidney, placenta, and choroid plexus, whereas FR beta is expressed in placenta, spleen, and thymus. Importantly, FR alpha is overexpressed in a number of carcinomas including up to 90% of ovarian cancers. Close associations were reported between FR alpha expression levels with grade and differentiation status of ovarian tumors. FR alpha in normal tissues (unlike tumors) is reported to be inaccessible to the circulation. FR beta is expressed in a wide range of myeloid leukemia cells. FR beta in normal hematopoetic cells differs from that in leukemia cells in its inability to bind folate ligand.

Folate-conjugated cytotoxins, liposomes, or radionuclides, or cytoxic antifolates have all been used to target FRs. Unfortunately, for most folate-based therapeutics such as classical antifolates (including RTX, PMX, and lometrexol (LMX)), tumor selectivity is lost since substrates are shared between FRs and the ubiquitously expressed RFC. Indeed, this likely explains the severe myelosupression encountered in phase 1 studies with LMX.

If, a FR-targeted ligand were itself cytotoxic without RFC activity, selective tumor targeting would ensue. Antifolates that selectively target FRs over RFC have been described including CB3717 and, more recently, cyclopenta[g]quinazoline antifolates BGC638 and BGC945, all of which potently inhibit thymidylate synthase (TS) within cells. When BGC945 was tested in mice, there was no toxicity to normal tissues, as reflected in weight loss, nor were there any macroscopic signs of toxicity to major organs, consistent with the premise that FR targeting is highly selective.

As is known by those skilled in the art, FRs such as FR alpha and FR beta are overexpressed on a substantial amount of certain surfaces of a number of types of cancerous tumors. FR alpha is known to be overexpressed in ovarian, endometrial, kidney, lung, mesothelioma, breast and brain tumors. FR beta is known to be overexpressed in acute myeloid leukemias

In most normal cells, the FRs are not present. In most normal cells, folic acid is not taken up by way of a reduced folate carrier (RFC) system. Uptake of folates and antifolates by tissues and tumors is primarily by the ubiquitously expressed RFC system. In light of the specificity of folic acid, conjugates of folic acid have been used to selectively deliver toxins, liposomes, imaging agents, and cytotoxic agents to FR expressing tumors. The major limitation of the folic acid conjugates is that they require cleavage from the folic acid moiety to release, for example, the cytotoxic agent. Cleavage of the cytotoxic agent moiety from the folic acid conjugate is often difficult to achieve and the anti-tumor activity is hindered or is nonexistent as a result of the inability or reduced ability to release the cytotoxic agent. Another limitation of the folic acid conjugates entails premature release of the cytotoxic agent during transport and before reaching the cancerous tumor. The premature release thus leads to undesired toxicity in normal cells.

The FRs alpha and beta represent another mode of folate uptake and are considered by those skilled in the art to be potential chemotherapeutic targets for selective tumor uptake. US Patent Application Publication No. US 2008/0045710 A1, published Feb. 21, 2008 (Aleem Gangjee) describes compounds for treating cancer tumors wherein fused cyclic pyrimidines are used to selectively target FRs of cancerous tumors that express FR alpha and FR beta and that inhibit glycinamide ribonucleotide formyltransferase (GARFTase) enzyme. The compounds are not significantly taken up by a cell or tissue using the RFC system.

There is a need for single compounds having potent anti-tumor activity that selectively target FR alpha and FR beta of cancerous cells, that inhibit GARFTase in cancerous cells, and that have a negligible substrate activity for RFC.

### SUMMARY OF THE INVENTION

The present invention meets the above need by providing selective proton coupled folate transporter (PCFT) and alpha and beta FR, and GARFTase enzyme and/or other folate metabolizing enzymes inhibitor compounds. Other folate metabolizing enzymes are such as for example but not limited to, thymidylate synthase (TS), dihydrofolate reductase (DHFR), and AICARFTase (5-amino-4-imidazole carboxamide ribonucleotide formyltransferase)

The present invention provides a compound comprising Formula I:

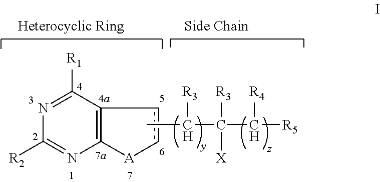

wherein R₁ comprises one of (a) a hydrogen (H)), (b) an OH, (c) CH₃, and (d) NHR wherein R is either a H or an alkyl group having from 1 to 6 carbon atoms, and tautomers of (b) and (d); R₂ comprises one of (a) a hydrogen (H), (b) a CH₃, (c) an OH, and (d) NHR wherein R is either a H or an alkyl group having from 1 to 6 carbon atoms; A comprises one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 may either be a single or a double bond; wherein the five membered ring has a side chain attached at positions 5, 6 or 7, and wherein when said side chain attachment is at position 7 then A comprises one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof; and R₃ comprises one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group, then R₄ is a hydrogen or a bond; wherein R₅ is the same as R₃ except that R₅ is not a bond; y is an integer ranging from zero up to and including 7; z is an integer ranging from zero up to and including seven, wherein the sum total of integers y and z is equal to or less than seven.

Another embodiment of this invention comprises the compound of Formula I, as described herein, wherein the side chain attachment is at carbon atom position 6 and wherein A is CR′R″, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, the compound of Formula I, as described herein, is provided comprising wherein the side chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In yet another embodiment of this invention, a compound of Formula I, as described herein, is provided comprising wherein said side chain attachment is at carbon atom position 5 and wherein and wherein A is CR′R″, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

Another embodiment of this invention provides a compound of Formula I, as described herein, comprising wherein the side chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, the compound of Formula I, as described herein, comprises the side chain having one or more carbon to carbon double or triple bonds between the carbon atoms of (C)_{y} and (C)_{z}.

In a preferred embodiment of this invention, the compound of Formula I, as described herein, is provided comprising wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group. The heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof. The heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

In another embodiment of this invention, the compound of Formula I, as described herein, provides wherein the side chain of Formula I comprises zero or one or more double bonds comprising E-isomers and Z-isomers.

Other embodiments of this invention provide for the R and S optical isomers of the heterocyclic compounds of the present invention when the double bond of the ring system is broken.

Other embodiments of this invention provide a pharmaceutical composition having a therapeutically effective amount of a compound comprising Formula I, and a pharmaceutically acceptable salt, prodrug, solvate or hydrate of the compound comprising Formula I, as described herein.

Further embodiments of this invention provide methods for treating cancer, targeting cancerous cells via the proton coupled folate transporter pathway, inhibiting GARFTase in cancerous cells, and selectively targeting activated macrophages in a patient having an autoimmune disease, such as rheumatoid arthritis.

A preferred embodiment of the present invention provides for a compound comprising Formula II:

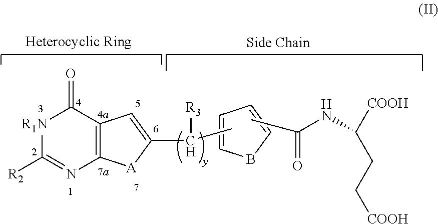

wherein R₁ is one of a hydrogen (H) or an alkyl group having from 1 to 6 carbon atoms;

R₂ is one of (a) a hydrogen (H), (b) a CH₃, (c) an OH, and (d) NHR wherein R is either a H or an alkyl group having from 1 to 6 carbon atoms;

A is one of (a) CR′R″, (b) NR′, wherein R′ and R″ are the same or different and are either a H or an alkyl group having from 1 to 6 carbon atoms, (c) a sulfur (S), and (d) an oxygen (O);

wherein the bond at position 5-6 is a double bond;

wherein the five membered ring of the Heterocyclic Ring of Formula II has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof; and

R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;

B is one of (a) a sulfur (S) atom, (b) an oxygen (O) atom, (c) CH₂, or (d) a NR′; and

y is an integer ranging from zero up to and including 8, and

wherein the (CH)_{y} of the Side Chain of Formula II is attached to the five membered ring of the Side Chain of Formula II at any one of positions 2, 3, 4, and 5 of the five membered ring of the Side Chain of Formula II (numbering clockwise from element B as position I of the five membered ring of the Side Chain of Formula II), and wherein the carbonyl-L-glutamate substituent of the Side Chain of Formula II is attached to the five membered ring of the Side Chain of Formula II at any one of the positions 2, 3, 4, and 5, except that the (CH)_{y} and the carbonyl-L-glutamate are attached at different positions of the five membered ring of the Side Chain of Formula II (i.e. such that the CH_{y} group and the carbonyl-L-glutamate substituent never occupy the same position in any given combinations).

In another embodiment of the present invention, the compound of Formula II, as described herein includes wherein the Side Chain of Formula II has one or more carbon to carbon double or triple bonds between the carbon atoms of (C)_{y 1-8}.

Another embodiment of the present invention provides the compound of Formula II including wherein the Side chain of Formula II comprises zero or one or more double bonds comprising E-isomers and Z-isomers.

Yet other embodiments of the present invention provide for a pharmaceutically acceptable salt, prodrug, solvate, or hydrate of the compound of Formula II.

Another embodiment of this invention provides the compounds of Formula II including tautomers of the Heterocyclic Ring of Formula II. These tautomers include such as for example the keto-enol form, or a lactam-lactim form of the compounds.

Other embodiments of the present invention provide the compounds of Formula II including positional regioisomers, geometric isomers, optical isomers, and conformational isomers of Formula II.

Another embodiment of the present invention includes a compound comprising Formula III:

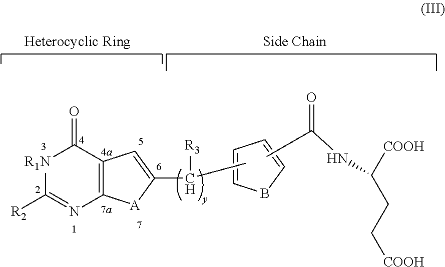

wherein R₁ comprises one of a hydrogen (H) or an alkyl group having from 1 to 6 carbon atoms; R₂ comprises one of (a) a hydrogen (H), (b) a CH₃, (c) an OH, and (d) NHR wherein R is either a H or an alkyl group having from 1 to 6 carbon atoms; A comprises one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 is a double bond; wherein the five membered ring of the Heterocyclic Ring of Formula III has a side chain attached at position 6, and optionally includes wherein the carbon atoms at positions 5 and 6, independently, have attached thereto either (a) one hydrogen atom, or (b) an alkyl group having from one to six carbon atoms, and combinations thereof, and R₃ comprises one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; B is one of (a) a sulfur (S) atom, (b) an oxygen (O) atom, (c) CH₂, or (d) a NR′; and y is an integer ranging from zero up to and including 8; and wherein the (CH)_{y} of the Side Chain of Formula III is attached to the five membered ring of the Side Chain of Formula III at any one of positions 2, 3, 4, and 5 of the five membered ring of the Side Chain of Formula III (numbering clockwise from element B as position 1 of the five membered ring of the Side Chain of Formula III), and wherein the carbonyl-L-glutamate substituent of the Side Chain of Formula III is attached to the five membered ring of the Side Chain of Formula III at any one of said positions 2, 3, 4, and 5, except that the (CH)_{y} and the carbonyl-L-glutamate are attached at different positions of the five membered ring of the Side Chain of Formula III (i.e. such that the CH_{y} group and the carbonyl-L-glutamate substituent never occupy the same position in any given combinations).

Other embodiments of the present invention provide for the compounds of Formula III wherein the Side Chain has one or more carbon to carbon double or triple bonds between the carbon atoms of (C)_{y 1-8}; wherein the Side Chain of Formula III comprises zero or one or more double bonds comprising E-isomers and Z-isomers; wherein a pharmaceutically acceptable salt, prodrug, solvate, or hydrate of Formula III is provided; wherein tautomers of the Heterocyclic Ring of Formula III are provided, which include for example but not limited to the keto-enol form, or a lactam-lactim form of Formula III; wherein positional regioisomers, geometric isomers, optical isomers, and conformational isomers of Formula III are provided; and wherein a pharmaceutical composition comprising a therapeutically effective amount of a compound of Formula III is provided.

Another embodiment of the present invention provides a compound comprising Formula IV:

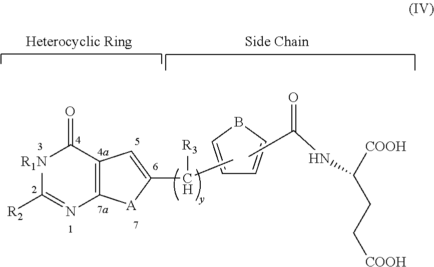

wherein R₁ is one of a hydrogen (H) or an alkyl group having from 1 to 6 carbon atoms; R₂ comprises one of (a) a hydrogen (H), (b) a CH₃, (c) an OH, and (d) NHR wherein R is either a H or an alkyl group having from 1 to 6 carbon atoms; A is one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 is a double bond;

wherein the five membered ring of the Heterocyclic Ring of Formula IV has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof, and R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; B is one of (a) a sulfur (S) atom, (b) an oxygen (O) atom, (c) CH₂, or (d) a NR′; and y is an integer ranging from zero up to and including 8; and wherein the (CH)_{y} of the Side Chain of Formula IV is attached to the five membered ring of the Side Chain of Formula IV at any one of positions 2, 3, 4, and 5 of the five membered ring of the Side Chain of Formula IV (numbering clockwise from element B as position 1 of the five membered ring of the Side Chain of Formula IV), and wherein the carbonyl-L-glutamate substituent of the Side Chain of Formula IV is attached to the five membered ring of the Side Chain of Formula IV at any one of said positions 2, 3, 4, and 5, except that said (CH)_{y} and the carbonyl-L-glutamate are attached at different positions of the five membered ring of the Side Chain of Formula IV (i.e. such that the CH_{y} group and the carbonyl-L-glutamate substituent never occupy the same position in any given combinations).

Other embodiments of the present invention provide wherein the compound of Formula IV includes wherein the Side Chain has one or more carbon to carbon double or triple bonds between the carbon atoms of (C)_{y 1-8}; wherein the Side Chain of Formula IV has zero or one or more double bonds comprising E-isomers and Z-isomers; wherein one of a pharmaceutically acceptable salt, prodrug, solvate, or hydrate of the compound of Formula IV is provided; wherein tautomers of the Heterocyclic Ring of the compound of Formula IV are provided, including such as for example the keto-enol form, or a lactam-lactim form of the compounds of Formula IV; wherein positional regioisomers, geometric isomers, optical isomers, and conformational isomers of the compounds of Formula IV are provided; and wherein a pharmaceutical composition comprising a therapeutically effective amount of a compound of Formula IV is provided.

In yet another embodiment of the present invention, a compound is provided comprising Formula V:

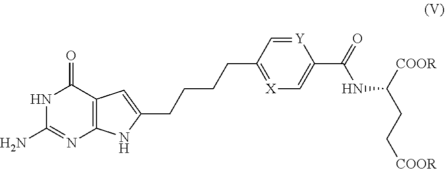

wherein X is N or CH; Y is N when X is CH and Y is CH when X is N; and

R is H. Other embodiments provide a pharmaceutically acceptable salt, prodrug, solvate, or hydrate of the compound of Formula V; tautomers of the heterocyclic ring of the compound of Formula V, including such as for example but not limited to tautomers of the keto-enol form, or a lactam-lactim form; positional regioisomers, geometric isomers, optical isomers, and conformational isomers of the compound of Formula V; and a pharmaceutical composition comprising a therapeutically effective amount of a compound of Formula V.

Another embodiment provides a compound comprising Formula VI:

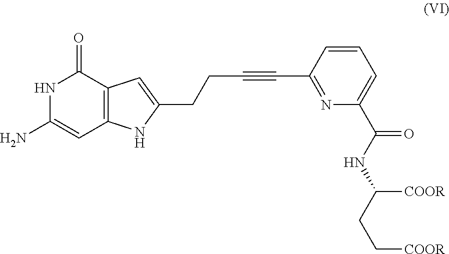

wherein R is H. Other embodiments provide a pharmaceutically acceptable salt, prodrug, solvate, or hydrate of the compound of Formula V; tautomers of the heterocyclic ring of Formula VI, including for example but not limited to tautomers of a keto-enol form, or a lactam-lactim form; positional regioisomers, geometric isomers, optical isomers, and conformational isomers of the compound of Formula VI; and a pharmaceutical composition comprising a therapeutically effective amount of a compound of Formula VI.

Another embodiment provides for a compound comprising Formula VII:

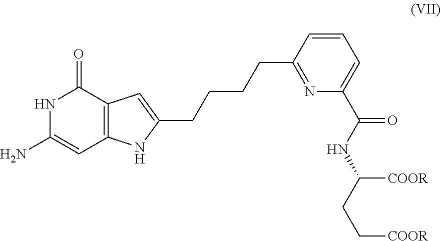

wherein R is H. Other embodiments provide for a pharmaceutically acceptable salt, prodrug, solvate, or hydrate of the compound of Formula VII; tautomers of the heterocyclic ring of the compounds of Formula VII, including such as for example but not limited to tautomers of a keto-enol form, or a lactam-lactim form; positional regioisomers, geometric isomers, optical isomers, and conformational isomers of the compounds of Formula VII; and a pharmaceutical composition comprising a therapeutically effective amount of a compound of Formula VII.

In yet another embodiment of this invention, a compound comprising Formula VIII:

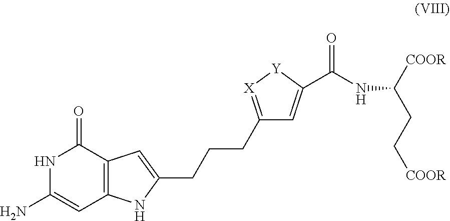

wherein X is CH or S; wherein Y is S when X is CH and Y is CH when X is S; and wherein R is H. Other embodiments include a pharmaceutically acceptable salt, prodrug, solvate, or hydrate of the compounds of Formula VIII; tautomers of the heterocyclic ring of the compound of Formula VIII, including such as for example but not limited to the keto-enol form, or a lactam-lactim form; positional regioisomers, geometric isomers, optical isomers, and conformational isomers of the compounds of Formula VIII; and pharmaceutical composition comprising a therapeutically effective amount of a compound of Formula VIII.

Another embodiment provides a compound comprising Formula IX:

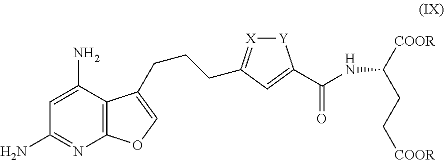

wherein X is CH or S; wherein Y is S when X is CH and Y is CH when X is S; and wherein R is H. Other embodiments provide a pharmaceutically acceptable salt, prodrug, solvate, or hydrate of the compounds of Formula IX; tautomers of the heterocyclic ring of the compound of Formula IX, including such as for example but not limited to tautomers of a keto-enol form, or a lactam-lactim form; positional regioisomers, geometric isomers, optical isomers, and conformational isomers of the compounds of Formula IX; and a pharmaceutical composition comprising a therapeutically effective amount of a compound of Formula IX.

Another embodiment of the present invention provides a compound comprising Formula X:

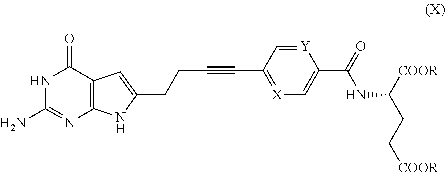

wherein X is N or CH; wherein Y is CH when X is N and wherein Y is N when X is CH; and wherein R is H. Other embodiments provide a pharmaceutically acceptable salt, prodrug, solvate, or hydrate of the compounds of Formula X; tautomers of the heterocyclic ring of the compound of Formula X, including such as for example but not limited to tautomers of a keto-enol form, or a lactam-lactim form; positional regioisomers, geometric isomers, optical isomers, and conformational isomers of the compounds of Formula X; and a pharmaceutical composition comprising a therapeutically effective amount of a compound of Formula X.

### BRIEF DESCRIPTION OF THE DRAWINGS

A full understanding of the invention may be gained from the following description of the preferred embodiments of the when read in conjunction with the accompanying drawings in which:

FIG. 1 shows the chemical structures of examples of the compounds of the present invention, namely, sample IDs G104, G106, G108, G119, G120, G121, G122, and G123.

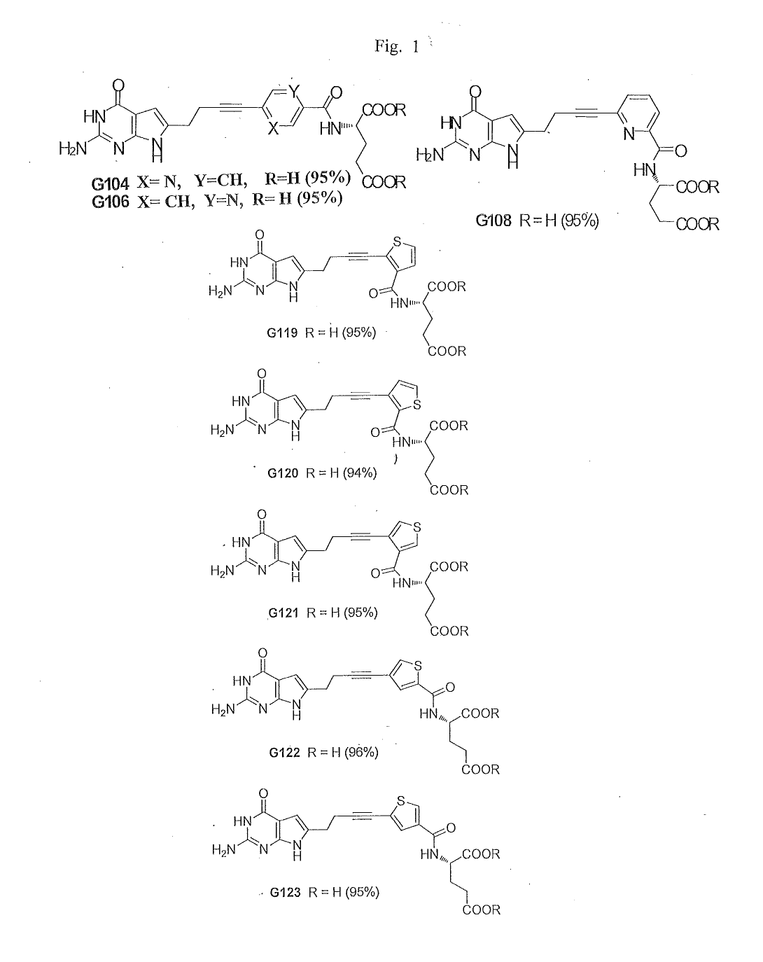

FIG. 2 shows the chemical structures of examples the compounds of the present invention, namely, sample IDs G105, G107, G109, G114, G115, G116, G117, and 0118.

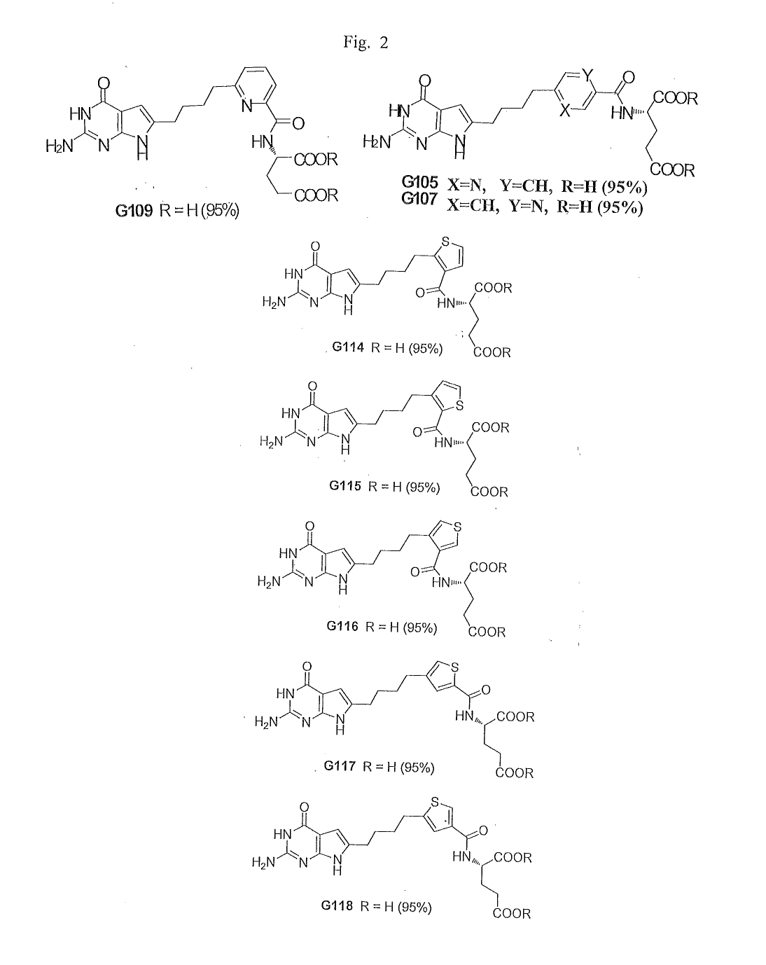

FIG. 3 shows the chemical structures of examples of the compounds of the present invention, namely, sample IDs G150 and G154.

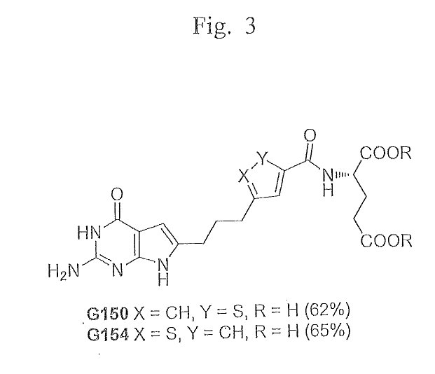

FIG. 4 shows the chemical structures of examples of the compounds of the present invention, namely, sample IDs G152 and G155.

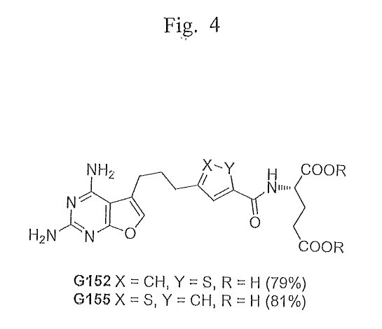

FIG. 5 shows the biological effects of compounds G104-G109 of the instant invention on hRFC, hPCFT, and FR-Expressing cell lines.

FIG. 6 shows the biological effects of compounds G114-G123 of the instant invention on hRFC, hPCFT, and FR-Expressing cell lines.

FIG. 7 shows the biological effects of compounds G150, G152, G154, and G155 of the instant invention on hRFC, hPCFT, and FR-Expressing cell lines.

### DESCRIPTION OF THE PREFERRED EMBODIMENTS

The present invention provides a compound comprising Formula I:

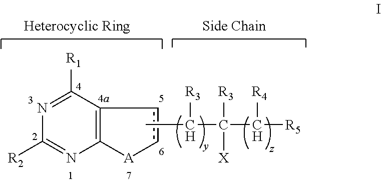

wherein R₁ comprises one of (a) a hydrogen (H)), (b) an OH, (c) CH₃, and (d) NHR wherein R is either a H or an alkyl group having from 1 to 6 carbon atoms, and tautomers of (b) and (d); R₂ comprises one of (a) a hydrogen (H), (b) a CH₃, (c) an OH, and (d) NHR wherein R is either a H or an alkyl group having from 1 to 6 carbon atoms; A comprises one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 may either be a single or a double bond; wherein the five membered ring has a side chain attached at positions 5, 6 or 7, and wherein when said side chain attachment is at position 7 then A comprises one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof; and R₃ comprises one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond; wherein R₅ is the same as R₃ except that R₅ is not a bond; y is an integer ranging from zero up to and including 7; z is an integer ranging from zero up to and including seven, wherein the sum total of integers y and z is equal to or less than seven.

Another embodiment of this invention comprises the compound of Formula I, as described herein, wherein the side chain attachment is at carbon atom position 6 and wherein A is the CR′R″, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, the compound of Formula I, as described herein, is provided comprising wherein the side chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In yet another embodiment of this invention, a compound of Formula I, as described herein, is provided comprising wherein said side chain attachment is at carbon atom position 5 and wherein and wherein A is the CR′R″, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

Another embodiment of this invention provides a compound of Formula I, as described herein, comprising wherein the side chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

The heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

The heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

In another embodiment of this invention, the compound of Formula I, as described herein, comprises the side chain having one or more carbon to carbon double or triple bonds between the carbon atoms of (C)_{y} and (C)_{z}.

In a preferred embodiment of this invention, the compound of Formula I, as described herein, is provided comprising wherein A is NR′ and R′ is a hydrogen atom, and wherein y is from one to seven carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

In another embodiment of this invention, the compound of Formula I, as described herein, provides wherein the Side Chain of Formula I comprises zero or one or more double bonds comprising E-isomers and Z-isomers.

Another embodiment of this invention provides a pharmaceutically acceptable salt, prodrug, solvate or hydrate of the compound of Formula I, as described herein.

In yet another embodiment of this invention, a pharmaceutical composition is provided comprising a therapeutically effective amount of a compound comprising Formula I:

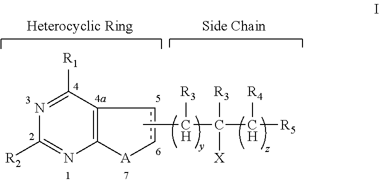

wherein R₁ is one of (a) a hydrogen (H)), (b) an OH, (c) CH₃, and (d) NHR wherein R is either a H or an alkyl group having from 1 to 6 carbon atoms, and tautomers of (b) and (d); R₂ is one of (a) a hydrogen (H), (b) a CH₃, (c) an OH, and (d) NHR wherein R is either a H or an alkyl group having from 1 to 6 carbon atoms; A is one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 may either be a single or a double bond; wherein the five membered ring has a side chain attached at positions 5, 6 or 7, and wherein when said side chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof; and R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; X is either a heterocycloalkyl-carbonyl-L-glutamate group or a heterocycloaryl-carbonyl-L-glutamate group or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond; wherein R₅ is the same as R₃ except that R₅ is not a bond; y is an integer ranging from zero up to and including 7; z is an integer ranging from zero up to and including seven, wherein the sum total of integers y and z is equal to or less than seven.

In another embodiment of this invention, the pharmaceutical composition comprises wherein the side chain attachment is at carbon atom position 6 and wherein A is CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, the pharmaceutical composition of Formula I comprises wherein the side chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

Another embodiment of this invention provides the pharmaceutical composition of Formula I comprising wherein the side chain attachment is at carbon atom position 5 and wherein and wherein A is CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

A further embodiment of this invention provides the pharmaceutical composition of Formula I comprising wherein the side chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

Another embodiment of this invention provides the pharmaceutical composition of Formula I comprising the side chain having one or more carbon to carbon double or triple bonds between the carbon atoms of (C)_{y} and (C)_{z}.

In a preferred embodiment of this invention, the pharmaceutical composition of Formula I comprises wherein A is NR′ and R′ is a hydrogen atom, and wherein y is from one to seven carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

In another embodiment of this invention, the pharmaceutical composition of Formula I comprises wherein said the side chain of Formula I comprises zero or one or more double bonds comprising E-isomers and Z-isomers.

This invention provides for a pharmaceutically acceptable salt, prodrug, solvate or hydrate of the pharmaceutical composition of Formula I, as described herein.

A method of treating a patient diagnosed with cancer is provided in this invention comprising administering to a patient a therapeutically effective amount of a compound of Formula I:

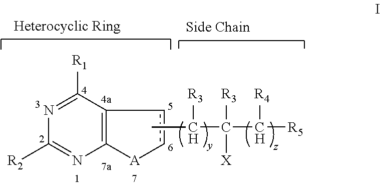

wherein R₁ is one of (a) a hydrogen (H)), (b) an OH, (c) CH₃, and (d) NHR wherein R is either a H or an alkyl group having from 1 to 6 carbon atoms, and tautomers of (b) and (d); R₂ is one of (a) a hydrogen (H), (b) a CH₃, (c) an OH, and (d) NHR wherein R is either a H or an alkyl group having from 1 to 6 carbon atoms; A is one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 may either be a single or a double bond; wherein the five membered ring has a side chain attached at positions 5, 6 or 7, and wherein when said side chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof; and R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond; wherein R₅ is the same as R₃ except that R₅ is not a bond; y is an integer ranging from zero up to and including 7; and z is an integer ranging from zero up to and including seven, wherein the sum total of integers y and z is equal to or less than seven.

In another embodiment of this invention, the method of treating a patient with cancer, as described herein, includes administering to the patient a compound of Formula I comprising wherein the side chain attachment is at carbon atom position 6 and wherein A is CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

Another embodiment of this invention provides a method of treating a patient with cancer, as described herein, including administering to the patient a compound of Formula I comprising wherein the side chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, a method of treating a patient with cancer, as described herein, includes administering to the patient a compound of Formula I wherein the side chain attachment is at carbon atom position 5 and wherein A is CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

In another embodiment of this invention, a method of treating a patient with cancer, as described herein, includes administering to a patient a compound of Formula I wherein the side chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

The methods of treating a patient with cancer, as described herein, include wherein the heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof, and wherein the heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

The methods of treating a patient with cancer, as described herein, include administering to the patient an effective amount of the compound of Formula I wherein the side chain has one or more carbon to carbon double or triple bonds between the carbon atoms of (C)_{y} and (C)_{z}.

Preferably, the method of treating a patient with cancer, as described herein, includes administering to the patient an effective amount of the compound of Formula I wherein A is NR′ and R′ is a hydrogen atom, and wherein y is from one to seven carbon atoms, z is zero, R₃, and R₅ are each hydrogen atoms, and X is selected from the group consisting of a heterocycloalkyl-carbonyl-L-glutamate group or a heterocycloaryl-carbonyl-L-glutamate group, as described herein. The method of treating a patient with cancer, as described herein, includes administering to the patient an effective amount of a compound of Formula I wherein the side chain of Formula I comprises zero or one or more double bonds comprising E-isomers and Z-isomers.

All of the methods of treating a patient with cancer, as described herein, include administering to the patient an effective amount of the Compound of Formula I, as described herein, or a pharmaceutically acceptable salt, prodrug, solvate or hydrate of the compound of Formula I, as described herein.

A method for targeting cancerous cells via the proton coupled folate transporter pathway is provided comprising:

(a) providing a compound comprising Formula I:

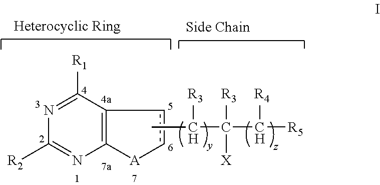

wherein R₁ is one of (a) a hydrogen (H)), (b) an OH, (c) CH₃, and (d) NHR wherein R is either a H or an alkyl group having from 1 to 6 carbon atoms, and tautomers of (b) and (d);

R₂ is one of (a) a hydrogen (H), (b) a CH₃, (c) an OH, and (d) NHR wherein R is either a H or an alkyl group having from 1 to 6 carbon atoms;

A is one of (a) CR′R″, (b) NR′, wherein R′ and R″ are the same or different and are either a H or an alkyl group having from 1 to 6 carbon atoms, (c) a sulfur (S), and (d) an oxygen (O);

wherein the bond at position 5-6 may either be a single or a double bond;

wherein the five membered ring has a side chain attached at positions 5, 6 or 7, and wherein when said side chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and

R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;

X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;

wherein R₅ is the same as R₃ except that R₅ is not a bond;

y is an integer ranging from zero up to and including 7;

z is an integer ranging from zero up to and including seven, wherein the sum total of integers y and z is equal to or less than seven;

(b) subjecting cancerous cells expressing a human proton coupled folate transporter (PCFT) to said compound of Formula I;

(c) establishing selective binding of said compound of Formula Ito said human PCFT; and

(d) effecting the selective transport of said compound of Formula I bound to said human PCFT to a target cancerous cell wherein said compound of Formula I acts as a growth inhibitor of said target cancerous cells and inhibits GARFTase within said target cancerous cells.

Another embodiment of this method for targeting cancerous cells of this invention, as described herein, include wherein the compound of Formula I is selective for receptors of FR alpha and human PCFT associated with expressing cancerous cells. In this method of targeting cancerous cell, the compound of Formula I is not significantly taken up by tissues or cells using the reduced folate carrier (RFC) system.

Other embodiments of this method for targeting cancerous cells comprise employing any of the various compounds of Formula I, or a pharmaceutically acceptable salt, prodrug, solvate or hydrate of a compound of Formula I, as described herein, thus it will be understood by those skilled in the art that any of the positions for attaching the side chain, as described herein, are embodiments of this invention. These methods for targeting cancer cells include wherein the compound targets cancerous cells selected from the group consisting of ovarian, breast, cervical, and kidney brain tumors.

A method for inhibiting GARFTase in cancerous cells is provided comprising:

(a) providing a compound of Formula I having a cytotoxic affect:

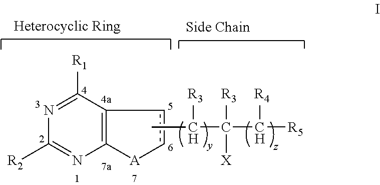

wherein R₁ is one of (a) a hydrogen (H)), (b) an OH, (c) CH₃, and (d) NHR wherein R is either a H or an alkyl group having from 1 to 6 carbon atoms, and tautomers of (b) and (d);

R₂ is one of (a) a hydrogen (H), (b) a CH₃, (c) an OH, and (d) NHR wherein R is either a H or an alkyl group having from 1 to 6 carbon atoms;

A is one of (a) CR′R″, (b) NR′, wherein R′ and R″ are the same or different and are either a H or an alkyl group having from 1 to 6 carbon atoms, (c) a sulfur (S), and (d) an oxygen (O);

wherein the bond at position 5-6 may either be a single or a double bond;

wherein the five membered ring has a side chain attached at positions 5, 6 or 7, and wherein when said side chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and

R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;

X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;

wherein R₅ is the same as R₃ except that R₅ is not a bond;

y is an integer ranging from zero up to and including 7;

z is an integer ranging from zero up to and including seven, wherein the sum total of integers y and z is equal to or less than seven;

(b) selectively delivering said compound to said cancerous cell;

(c) effecting the entry of said compound into said cancerous cell;

(d) retaining said compound in said cancerous cell for a sufficient amount of time for effecting binding of said compound with a GARFTase enzyme; and

(e) lysing of said cancerous cell via said binding of said compound with said GARFTase enzyme and inhibiting the DNA replication of said cancerous cell.

Preferably, the method, of this invention, of inhibiting GARFTase, as described herein, comprises wherein the compound of Formula I or a pharmaceutically acceptable salt, prodrug, solvate or hydrate of the compound of Formula I is selective for receptors of FR alpha associated with expressing cancerous cells.

Other embodiments of this invention of inhibiting GARTase, as described herein, include employing any one of the various embodiments of the compound of Formula I or its pharmaceutically acceptable salt, prodrug, solvate or hydrate, as described herein, including comprising the side chain attachment at various positions 5, 6 or 7, as described herein.

Another embodiment of this invention provides for the inhibition of AICARFTase when A is equal to a sulfur atom in the compound of Formula I.

Rheumatoid arthritis is an autoimmune disease that affects the quality of life of millions of patients worldwide. Rheumatoid arthritis is characterized by inflammation of a patient's joints and destruction of the cartilage and bone of the patient. While the pathology of rheumatoid arthritis is complex, it is known to involve the infiltration and activation of immune cells along with the release of destructive inflammatory mediators into a patient's synovium of affected joints. Paulos, Chrystal M., et al., “Folate receptor-mediated targeting of therapeutic and imaging agents to activated macrophages in rheumatoid arthritis”, Advanced Drug Delivery Reviews, Vol, 56, pages 1205-1217 (2004), describe the discovery of folate receptor expression on activated macrophage cells in patient models (human and animal) with naturally occurring rheumatoid arthritis, and is incorporated herein by reference, specifically section 3, page 1208 and section 5, pages 1212-1214,

The present invention provides a method for selectively targeting activated macrophages in a patient having an autoimmune disease comprising:

(a) providing a compound comprising Formula I:

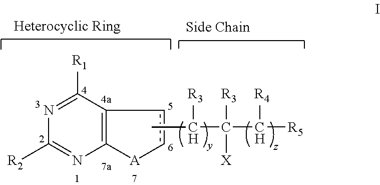

wherein R₁ is one of (a) a hydrogen (H), (b) an OH, (c) CH₃, and (d) NHR wherein R is either a H or an alkyl group having from 1 to 6 carbon atoms, and tautomers of (b) and (d);

R₂ is one of (a) a hydrogen (H), (b) a CH₃, (c) an OH, and (d) NHR wherein R is either a H or an alkyl group having from 1 to 6 carbon atoms;

A is one of (a) CR′R″, (b) NR′, wherein R′ and R″ are the same or different and are either a H or an alkyl group having from 1 to 6 carbon atoms, (c) a sulfur (S), and (d) an oxygen (O);

wherein the bond at position 5-6 may either be a single or a double bond;

wherein the five membered ring has a side chain attached at positions 5, 6 or 7, and wherein when said side chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and

R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;

X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;

wherein R₅ is the same as R₃ except that R₅ is not a bond;

y is an integer ranging from zero up to and including 7;

z is an integer ranging from zero up to and including seven, wherein the sum total of integers y and z is equal to or less than seven;

(b) subjecting an activated macrophage expressing a folate receptor (FR) to said compound of Formula I;

(c) establishing selective binding of said compound of Formula Ito said FR; and

(d) effecting the selective transport of said compound of Formula I bound to said FR to a target activated macrophage of the autoimmune disease wherein said compound of Formula I acts as an inhibitor of said activated macrophage's release of destructive inflammatory mediators.

The method for selectively targeting activated macrophages of the present invention includes wherein the compound of Formula I is selective for receptors of FR alpha and human proton coupled folate transporter (PCFT) associated with expressing macrophage cells.

Preferably, the method for selectively targeting activated macrophages in a patient having an autoimmune disease, as described herein, includes wherein the activated macrophage cell expressing the FR is rheumatoid arthritis.

Other embodiments of the method for targeting activated macrophage cells in a patient with an autoimmune disease, include wherein the compound of Formula I, or its pharmaceutically acceptable salts, prodrugs, solvates or hydrates of the compound of Formula I, include any of the various embodiments, as described herein, of the compound of Formula I, including attachment of the side chain at any of the positions 5, 6, or 7, as described herein.

Preferably, the method of selectively targeting an activated macrophage in a patient having an autoimmune disease that is rheumatoid arthritis includes delivering the compound of Formula I or a pharmaceutically acceptable salt, prodrug, solvate or hydrate of the compound of Formula I by injection into a joint or synovial fluid of a patient.

A preferred embodiment of the present invention provides for a compound comprising Formula II:

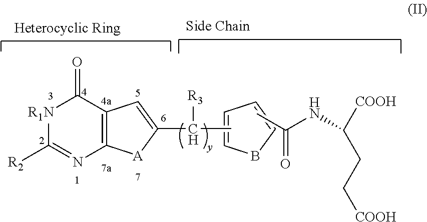

wherein R₁ is one of a hydrogen (H) or an alkyl group having from 1 to 6 carbon atoms;

R₂ is one of (a) a hydrogen (H), (b) a CH₃, (c) an OH, and (d) NHR wherein R is either a H or an alkyl group having from 1 to 6 carbon atoms;

A is one of (a) CR′R″, (b) NR′, wherein R′ and R″ are the same or different and are either a H or an alkyl group having from 1 to 6 carbon atoms, (c) a sulfur (S), and (d) an oxygen (O);

wherein the bond at position 5-6 is a double bond;

wherein the five membered ring of the Heterocyclic Ring has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof; and

R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;

B is one of (a) a sulfur (S) atom, (b) an oxygen (O) atom, (c) CH₂, or (d) a NR′; and

y is an integer ranging from zero up to and including 8, and

wherein the (CH)_{y} of the Side Chain of Formula II is attached to the five membered ring of the Side Chain of Formula II at any one of positions 2, 3, 4, and 5 of the five membered ring of the Side Chain of Formula II (numbering clockwise from element B as position 1 of the five membered ring of the Side Chain of Formula II), and wherein the carbonyl-L-glutamate substituent of the Side Chain of Formula II is attached to the five membered ring of the Side Chain of Formula II at any one of the positions 2, 3, 4, and 5, except that the (CH)_{y} and the carbonyl-L-glutamate are attached at different positions of the five membered ring of the Side Chain of Formula II (i.e. such that a carbon atom of the CH_{y} group and the carbon atom of the carbonyl-L-glutamate substituent attached to the five membered ring of the Side Chain are never each attached at the same position on the five membered ring in any given combinations). Formula II shows that the substituent attachments of the five membered ring of the Side Chain are in many possible positional combinations, such as for example but not limited to, at the 2 and 5 positions (numbering clockwise with “B” being at position 1), at the 2 and 3 positions, at the 2 and 4 positions, at the 3 and 4 positions, at the 3 and 5 positions, and at the 4 and 5 positions.

Another embodiment of this invention provides the compound of Formula II comprising wherein the side chain has one or more carbon to carbon double or triple bonds between the carbon atoms of (C)_{y 1-8}. In another embodiment of this invention the compound of Formula II comprises wherein the side chain comprises zero or one or more double bonds comprising E-isomers and Z-isomers. Another embodiment provides the compound of Formula II comprising one of a pharmaceutically acceptable salt, prodrug, solvate, or hydrate thereof. A pharmaceutical composition comprising a therapeutically effective amount of a compound of Formula II is also provided. Other preferred embodiments of the present invention provide for methods as described herein for treating cancer, selectively targeting cancerous cells via the proton coupled folate transporter, folate receptor alpha, and/or folate receptor beta pathways, inhibiting GARFTase in cancerous cells, and selectively targeting activated macrophages in a patient having an autoimmune disease employing the compounds of Formula II as the preferred tautomer provided by the compound of Formula I.

Another embodiment of this invention provides the compounds of Formula II including tautomers of the Heterocyclic Ring of Formula II. These tautomers include such as for example the keto-enol form, or a lactam-lactim form of the compounds.

Other embodiments of the present invention provide the compounds of Formula II including positional regioisomers, geometric isomers, optical isomers, and conformational isomers of Formula II.

Another embodiment of the present invention includes a compound comprising Formula III:

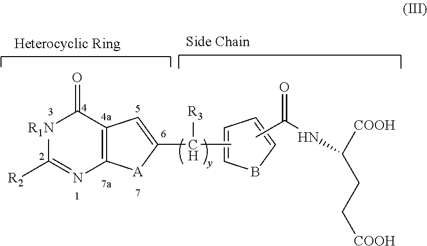

wherein R₁ is one of a hydrogen (H) or an alkyl group having from 1 to 6 carbon atoms;

R₂ is one of (a) a hydrogen (H), (b) a CH₃, (c) an OH, and (d) NHR wherein R is either a H or an alkyl group having from 1 to 6 carbon atoms; A is one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 is a double bond;

wherein the five membered ring of the Heterocyclic Ring of Formula III has a side chain attached at position 6, and optionally includes wherein the carbon atoms at positions 5 and 6, independently, have attached thereto either (a) one hydrogen atom, or (b) an alkyl group having from one to six carbon atoms, and combinations thereof, and R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; B is one of (a) a sulfur (S) atom, (b) an oxygen (O) atom, (c) CH₂, or (d) a NR′; and y is an integer ranging from zero up to and including 8; and wherein the (CH)_{y} of the Side Chain of Formula III is attached to the five membered ring of the Side Chain of Formula III at any one of positions 2, 3, 4, and 5 of the five membered ring of the Side Chain of Formula III (numbering clockwise from element B as position 1 of the five membered ring of the Side Chain of Formula III), and wherein the carbonyl-L-glutamate substituent of the Side Chain of Formula III is attached to the five membered ring of the Side Chain of Formula III at any one of said positions 2, 3, 4, and 5, except that the (CH)_{y} and the carbonyl-L-glutamate are attached at different positions of the five membered ring of the Side Chain of Formula III (i.e. such that a carbon atom of the CH_{y} group and the carbon atom of the carbonyl-L-glutamate substituent attached to the five membered ring of the Side Chain are never each attached at the same position on the five membered ring in any given combinations). Formula III shows that the substituent attachments of the five membered ring of the Side Chain are in many possible positional combinations, such as for example but not limited to, at the 2 and 5 positions (numbering clockwise with “B” being at position 1), at the 2 and 3 positions, at the 2 and 4 positions, at the 3 and 4 positions, at the 3 and 5 positions, and at the 4 and 5 positions.

Other embodiments of the present invention provide for the compounds of Formula III wherein the Side Chain has one or more carbon to carbon double or triple bonds between the carbon atoms of (C)_{y 1-8}; wherein the Side Chain of Formula III comprises zero or one or more double bonds comprising E-isomers and Z-isomers; wherein a pharmaceutically acceptable salt, prodrug, solvate, or hydrate of Formula III is provided; wherein tautomers of the Heterocyclic Ring of Formula III are provided, which include for example but not limited to the keto-enol form, or a lactam-lactim form of Formula III; wherein positional regioisomers, geometric isomers, optical isomers, and conformational isomers of Formula III are provided; and wherein a pharmaceutical composition comprising a therapeutically effective amount of a compound of Formula III is provided.

Another embodiment of the present invention provides a compound comprising Formula (IV):

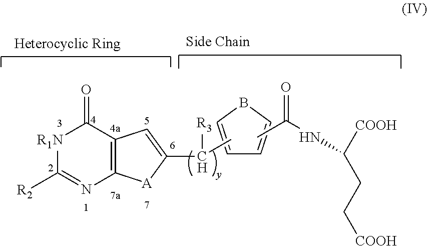

wherein R₁ is one of a hydrogen (H) or an alkyl group having from 1 to 6 carbon atoms; R₂ comprises one of (a) a hydrogen (H), (b) a CH₃, (c) an OH, and (d) NHR wherein R is either a H or an alkyl group having from 1 to 6 carbon atoms; A is one of (a) CR′R″, (b) NR′, wherein R′ and R″ are the same or different and are either a H or an alkyl group having from 1 to 6 carbon atoms, (c) a sulfur (S), and (d) an oxygen (O); wherein the bond at position 5-6 is a double bond;

wherein the five membered ring of the Heterocyclic Ring of Formula IV has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof, and R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond; B is one of (a) a sulfur (S) atom, (b) an oxygen (O) atom, (c) CH₂, or (d) a NR′; and y is an integer ranging from zero up to and including 8; and wherein the (CH)_{y} of the Side Chain of Formula IV is attached to the five membered ring of the Side Chain of Formula IV at any one of positions 2, 3, 4, and 5 of the five membered ring of the Side Chain of Formula IV (numbering clockwise from element B as position 1 of the five membered ring of the Side Chain of Formula IV), and wherein the carbonyl-L-glutamate substituent of the Side Chain of Formula IV is attached to the five membered ring of the Side Chain of Formula IV at any one of said positions 2, 3, 4, and 5, except that the (CH)_{y} and the carbonyl-L-glutamate are attached at different positions of the five membered ring of the Side Chain of Formula IV (i.e. such that a carbon atom of the CH_{y} group and the carbon atom of the carbonyl-L-glutamate substituent attached to the five membered ring of the Side Chain are never each attached at the same position on the five membered ring in any given combinations), Formula IV shows that the substituent attachments of the five membered ring of the Side Chain are in many possible positional combinations, such as for example but not limited to, at the 2 and 5 positions (numbering clockwise with “B” being at position 1), at the 2 and 3 positions, at the 2 and 4 positions, at the 3 and 4 positions, at the 3 and 5 positions, and at the 4 and 5 positions.

Other embodiments of the present invention provide wherein the compound of Formula IV includes wherein the Side Chain has one or more carbon to carbon double or triple bonds between the carbon atoms of (C)_{y 1-8}; wherein the Side Chain of Formula IV has zero or one or more double bonds comprising E-isomers and Z-isomers; wherein one of a pharmaceutically acceptable salt, prodrug, solvate, or hydrate of the compound of Formula IV is provided; wherein tautomers of the Heterocyclic Ring of the compound of Formula IV are provided, including such as for example the keto-enol form, or a lactam-lactim form of the compounds of Formula IV; wherein positional regioisomers, geometric isomers, optical isomers, and conformational isomers of the compounds of Formula IV are provided; and wherein a pharmaceutical composition comprising a therapeutically effective amount of a compound of Formula IV is provided.

In yet another embodiment of the present invention, a compound is provided comprising Formula V:

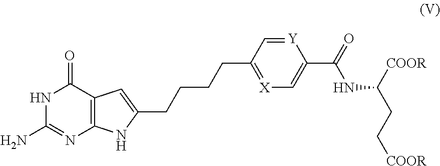

wherein X is N or CH; Y is N when X is CH and Y is CH when X is N; and R is H. Other embodiments provide a pharmaceutically acceptable salt, prodrug, solvate, or hydrate of the compound of Formula V; tautomers of the heterocyclic ring of the compound of Formula V, including such as for example but not limited to tautomers of the keto-enol form, or a lactam-lactim form; positional regioisomers, geometric isomers, optical isomers, and conformational isomers of the compound of Formula V; and a pharmaceutical composition comprising a therapeutically effective amount of a compound of Formula V.

Another embodiment provides a compound comprising Formula VI:

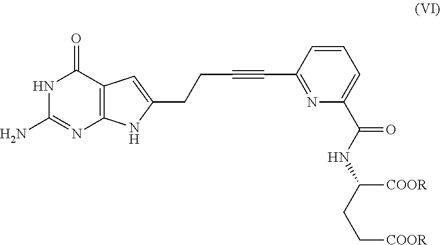

wherein R is H. Other embodiments provide a pharmaceutically acceptable salt, prodrug, solvate, or hydrate of the compound of Formula V; tautomers of the heterocyclic ring of Formula VI, including for example but not limited to tautomers of a keto-enol form, or a lactam-lactim form; positional regioisomers, geometric isomers, optical isomers, and conformational isomers of the compound of Formula VI; and a pharmaceutical composition comprising a therapeutically effective amount of a compound of Formula VI.

Another embodiment provides for a compound comprising Formula VII:

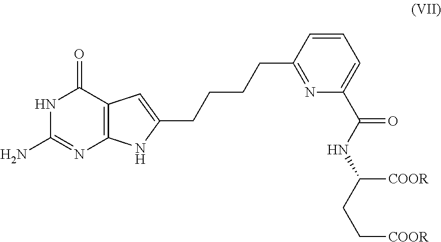

wherein R is H. Other embodiments provide for a pharmaceutically acceptable salt, prodrug, solvate, or hydrate of the compound of Formula VII; tautomers of the heterocyclic ring of the compounds of Formula VII, including such as for example but not limited to tautomers of a keto-enol form, or a lactam-lactim form; positional regioisomers, geometric isomers, optical isomers, and conformational isomers of the compounds of Formula VII; and a pharmaceutical composition comprising a therapeutically effective amount of a compound of Formula VII.

In yet another embodiment of this invention, a compound comprising Formula VIII:

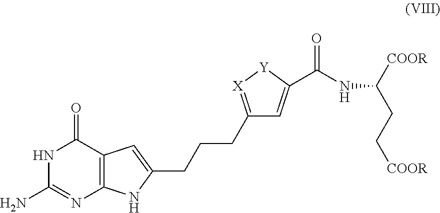

wherein X is CH or S; wherein Y is S when X is CH and Y is CH when X is S; and wherein R is H. Other embodiments include a pharmaceutically acceptable salt, prodrug, solvate, or hydrate of the compounds of Formula VIII; tautomers of the heterocyclic ring of the compound of Formula VIII, including such as for example but not limited to the keto-enol form, or a lactam-lactim form; positional regioisomers, geometric isomers, optical isomers, and conformational isomers of the compounds of Formula VIII; and pharmaceutical composition comprising a therapeutically effective amount of a compound of Formula VIII.

Another embodiment provides a compound comprising Formula IX:

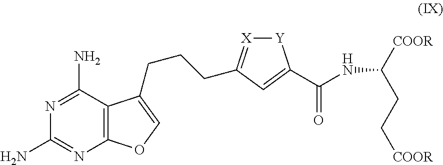

wherein X is CH or S; wherein Y is S when X is CH and Y is CH when X is S; and wherein R is H. Other embodiments provide a pharmaceutically acceptable salt, prodrug, solvate, or hydrate of the compounds of Formula IX; tautomers of the heterocyclic ring of the compound of Formula IX, including such as fro example but not limited to tautomers of a keto-enol form, or a lactam-lactim form; positional regioisomers, geometric isomers, optical isomers, and conformational isomers of the compounds of Formula IX; and a pharmaceutical composition comprising a therapeutically effective amount of a compound of Formula IX.

Another embodiment provides for a compound comprising Formula X:

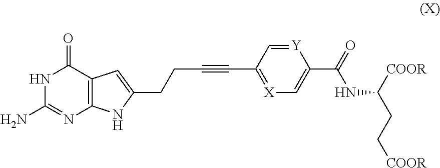

wherein X is N or CH; wherein Y is CH when X is N and Y is N when X is CH; and wherein R is H. Other embodiments provide a pharmaceutically acceptable salt, prodrug, solvate, or hydrate of the compounds of Formula X; tautomers of the heterocyclic ring of the compound of Formula X, including such as for example but not limited to tautomers of a keto-enol form, or a lactam-lactim form; positional regioisomers, geometric isomers, optical isomers, and conformational isomers of the compounds of Formula IX; and a pharmaceutical composition comprising a therapeutically effective amount of a compound of Formula X.

As used herein, the term “patient” means members of the animal kingdom, including, but not limited to, human beings. As used herein, the term “having cancer” means that the patient has been diagnosed with cancer.

As used herein, the term “therapeutically effective amount” refers to that amount of any of the present compounds required to bring about a desired effect in a patient. The desired effect will vary depending on the illness being treated. For example, the desired effect may be reducing tumor size, destroying cancerous cells, and/or preventing metastasis, any one of which may be the desired therapeutic response. On its most basic level, a therapeutically effective amount is that amount needed to inhibit the mitosis of a cancerous cell.

Compounds of the present invention covered under Formulae I through IX, and pharmaceutically acceptable salts, prodrugs, solvates or hydrates thereof, may also be administered with one or more additional treatment agents, i.e., a chemotherapeutic agent. Suitable candidates for the additional chemotherapeutic agent include for example but are not limited to, paclitaxel, docetaxel, vinca alkaloids, colchicines, colcemid, cisplatin, and nocadazol.

As used herein, the term “lower alkyl” group refers to those lower alkyl groups having one to about ten carbon atoms, such as for example methyl, ethyl, propyl, butyl, pentyl, hexyl, cyclopropyl, cyclobutyl, cyclohexyl, cyclopropylmethyl or cyclobutylmethyl groups. Alkyl groups sharing one to about six carbon atoms are preferred. These lower alkyl groups are straight chain, branched chain or cyclic (alicyclic hydrocarbon) arrangements. The carbon atoms of these straight chain, branched chain or cyclic arranged alkyl groups may have one or more substituents for the hydrogens attached to the carbon atoms.

As used herein, the term “heteroalkyl” refers to alkyl chains from one to about 3 atoms where one or more of the carbons has been replaced with nitrogen, oxygen or sulfur, Thus “heteroalkyl” groups will include, for example, C—C—N, C—S, S—C, C—O, C—C—O, O—C, N—C—C, N—C⁻C and other various combinations, as will be apparent to one skilled in the art. The above list is not meant to be exhaustive, and many combinations are contemplated as within the scope of the present invention.

The term “aryl” groups, as used herein, refers to compounds whose molecules have an aromatic ring structure, such as the six-carbon ring of benzene, or multiple rings which are either fused or unfused, such as condensed six-carbon rings of other aromatic derivatives. The term “aryl” is also defined to include diaryl, triaryl and polyaryl groups, which would have two, three or more rings, respectively. Thus, suitable aryl groups would include, for example, phenyl, biphenyl, naphthyl, phenanthrene, anthracene groups and aryl oxyaryl groups. This list is not meant to be exhaustive, and any aryl group, as these terms are defined above and commonly understood in the art, are within the scope of the present invention.

The term “heteroaryl” refers to aromatic ring structures having at least one atom in the ring which is not carbon, such as oxygen, nitrogen or sulfur. “Heteroaryls” as used herein also refers to aromatic ring structures that are part of larger ring structures, such as two or three member ring systems, which may be fused or unfused, in which one of the rings is as described above. Thus, “heteroaryl” refers to ring systems in which one or more rings contain a heteroatom and one or more rings do not. It will be understood that this list is not meant to be exhaustive, and that any heteroaryl group, as these terms are defined above and commonly understood in the art, are within the scope of the present invention. The heteroaryl ring systems may be fused ring systems or unfused. Examples of heteroaryl ring systems include, for example but are not limited to, pyridine, quinoline, isoquinoloine, pyrrole, thiophenes, furans, imidazoles, and the like, as well as fused ring structures having rings of different sizes, such as benzofurans, indoles, purines, and the like.

Also included within the scope of the present invention are alicyclic groups, as that term is understood in the art, and heterocyclic groups. As used herein, the term “heterocyclic group” refers to non-aromatic cyclic substituents in which one or more members of the ring is not carbon, for example oxygen, sulfur or nitrogen.

The terms “alkylaryl” (or “alkaryl”) or “alkylheteroaryl” as used herein refer to groups having an alkyl moiety attached to an aryl or heteroaryl ring. The alkyl moiety is preferably a straight, branched or cyclic alkyl group having one to about six carbon atoms. This alkyl moiety may also contain oxygen, nitrogen or sulfur, and therefore may be an alkoxy group. The aryl or heteroaryl moiety of the alkylaryl group is a substituted or unsubstituted aryl or heteroaryl group, as these terms are described above. As used herein, the terms “alkylaryl” or “alkylheteroaryl” will also be used to refer to arylalkyl groups or heteroarylalkyl groups, as those terms are understood in the art, and denotes attachment of such a substituent at either the alkyl or the aryl portion of the group. Thus, for example, a benzyl group would be embraced by the term “alkylaryl”.

Any of the cyclic substituents described above, such as the aryl, heteroaryl, alkylaryl, alkylheteroaryl, alicyclic, or heterocyclic groups are optionally substituted with one or more substituents as listed above. In the case of more than one substituent, the substituents are independently selected. “Alkoxy groups” and “alkyl groups” include straight or branched chains having up to about ten members. “Halogen” refers to chlorine, bromine, iodine and fluorine. “Aryl and heteroaryl groups” are as described above. When a carboxylic acid is a substituent, it will be appreciated that the moiety represents an acid such as benzoic acid. As used herein, the term heterocycloaryl-carbonyl-L-glutamate group may include for example a thiophene-carbonyl-L-glutamate group, a furan-carbonyl-L-glutamate group, a pyrrole-carbonyl-L-glutamate group, and a pyridine-carbonyl-L-glutamate group, and the term heterocycloalkyl-carbonyl-L-glutamate group may include for example a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof, as those terms are understood by one skilled in the art.

As used herein, the terms “aroyl” or “heteroaroyl”, such as when used within the term p-aroyl-L-glutamate, refers to benzoyl, napthoyl, thiophenoyl, furophenoyl, pyrroyl, and any other “aroyl” or “heteroaroyl” as these terms are understood by one skilled in the art, “Aroyl” and “heteroaroyl” are generally defined in the art as an aromatic or heteroaromatic compound having a carbonyl moiety. As used herein, the term “glutamate” will be understood as representing both the ester form (glutamate) and the acid form (glutamic acid).

Those skilled in the art shall understand that chemical structure of Formula II is a preferred example of this invention and that Formula II is a tautomer of an embodiment of a compound of Formula I. Those skilled in the art understand that chemical structures are often drawn as one tautomeric form over another. This invention provides for several tautomeric forms as covered by the description of Formulae I through X. The tautomeric forms taught by Formulae I through X of the present invention provide several structural embodiments that will be appreciated by those skilled in the art.

Proliferative diseases and/or disorders that may be treated according to the methods of the present invention include, without limitation, ovarian cancer, endometrial and cervical cancer, renal cancer, and breast cancer, and autoimmune diseases such as for example rheumatoid arthritis.

It is especially advantageous to formulate parenteral compositions in dosage unit form for ease of administration and uniformity of dosage. Dosage unit form as used herein refers to physically discrete units suited as unitary dosages for the patients being treated, each unit containing a predetermined quantity or effective amount of a compound of the present invention to produce the desired effect in association with a pharmaceutical carrier. The specification for the dosage unit forms of the invention are dictated by and directly dependent on the particular compound and the particular effect, or therapeutic response, that is desired to be achieved.

Compounds of Formulae I through X, or pharmaceutically acceptable salts, prodrugs, solvates, or hydrates thereof, can be administered to a patient (an animal or human) via various routes including parenterally, orally or intraperitoneally. Parenteral administration includes the following routes that are outside the alimentary canal (digestive tract): intravenous; intramuscular; interstitial, intraarterial; subcutaneous; intraocular; intracranial; intraventricular; intrasynovial; transepithelial, including transdermal, pulmonary via inhalation, ophthalmic, sublingual and buccal; topical, including dermal, ocular, rectal, or nasal inhalation via insufflation or nebulization. Specific modes of administration shall depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered to a patient shall depend on the characteristics of the patient being treated, including for example, but not limited to, the patient's age, weight, health, and types and frequency of concurrent treatment, if any, of any other chemotherapeutic agent(s), all of which is determined by the clinician as one skilled in the art.

Compounds of Formulae I through X, or a pharmaceutically acceptable salt, prodrug, solvate or hydrate thereof, that are orally administered can be enclosed in hard or soft shell gelatin capsules, or compressed into tablets. Compounds also can be incorporated with an excipient and used in the form of ingestible tablets, buccal tablets, troches, capsules, sachets, lozenges, elixirs, suspensions, syrups, wafers and the like. Compounds of Formulae I through X can be in the form of a powder or granule, a solution or suspension in an aqueous liquid or non-aqueous liquid, or in an oil-in-water emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. Additionally, the compounds of Formulae I-X, or a pharmaceutically acceptable salt, prodrug, solvate or hydrate of Formulae I through X, can be incorporated into sustained-release preparations and formulations.

The compounds of Formulae I through X, or a pharmaceutically acceptable salt, prodrug, solvate or hydrate thereof, can be administered to the central nervous system, parenterally or intraperitoneally. Solutions of the compound as a free base or a pharmaceutically acceptable salt can be prepared in water mixed with a suitable surfactant, such as hydroxypropylcellulose. Dispersions also can be prepared in glycerol, liquid polyethylene glycols and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include, without limitation, sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi.

Compounds of the present invention may be contained within, mixed with, or associated with, a suitable (acceptable) pharmaceutical carrier for administration to a patient according to the particular route of administration desired. Suitable or acceptable pharmaceutical carriers refer to any pharmaceutical carrier that will solubilize the compounds of the present invention and that will not give rise to incompatability problems, and includes any and all solvents, dispersion media, coatings, antibacterial and antifungal agents, isotonic agents, absorption delaying agents, and the like. The use of such suitable or acceptable pharmaceutical carriers are well known by those skilled in the art. Preferred carriers include sterile water, physiologic saline, and five percent dextrose in water. Examples of other suitable or acceptable pharmaceutical carriers include, but are not limited to, ethanol, polyol (such as propylene glycol and liquid polyethylene glycol), suitable mixtures thereof, or vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating a compound of Formulae I-X in the required amount in the appropriate solvent with various of the other ingredients enumerated above, as required, followed by filtered sterilization. Generally, dispersions are prepared by incorporating the sterilized compound of any of the Formulae I-X into a sterile vehicle that contains the basic dispersion medium and any of the other ingredients from those enumerated above. In the case of sterile powders for the preparation of sterile injectable solutions, the preferred methods of preparation are vacuum drying and freeze drying.

Pharmaceutical compositions which are suitable for administration to the nose and buccal cavity include, without limitation, self-propelling and spray formulations, such as aerosol, atomizers and nebulizers.

The therapeutic compounds of Formulae I through X, as described herein, can be administered to a patient alone or in combination with pharmaceutically acceptable carriers or as pharmaceutically acceptable salts, solvates or hydrates thereof, the proportion of which is determined by the solubility and chemical nature of the compound, chosen route of administration to the patient and standard pharmaceutical practice.

The present invention is more particularly described in the following non-limiting examples, which are intended to be illustrative only, as numerous modifications and variations therein will be apparent to those skilled in the art.

### Examples

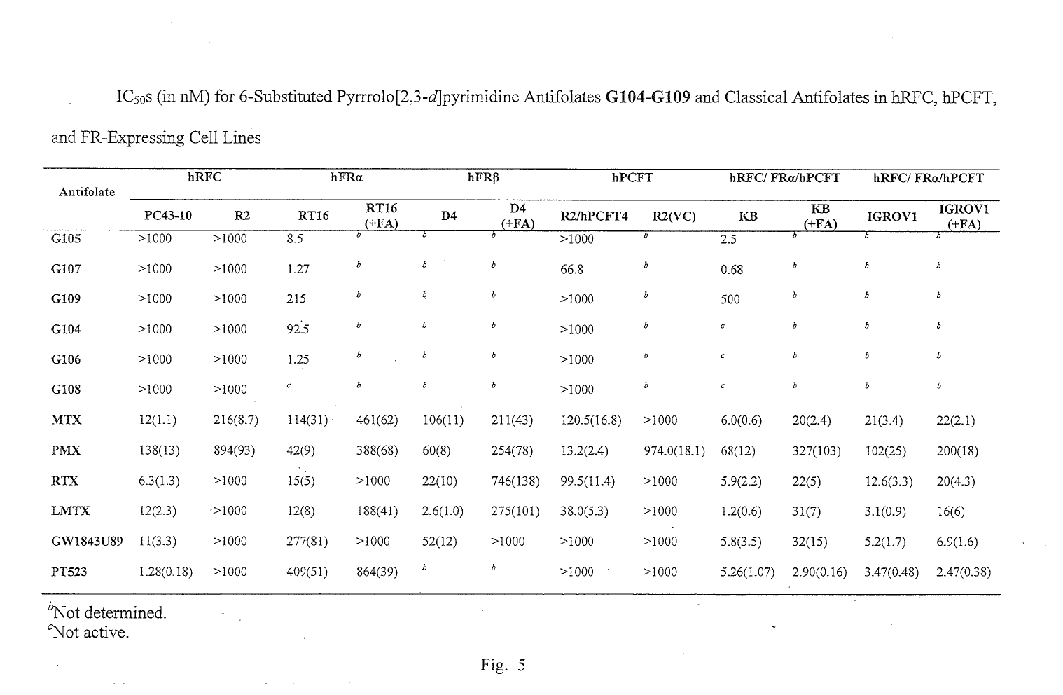

FIGS. 5, 6, and 7 show the biological effects of various compounds of the present invention, namely, Samples: G105, G107, G109, G104, G106, G108, G114-G123, and G150, G152, G154 and G155. These compounds were evaluated for cytotoxicity towards assorted cell lines, namely, KB human tumor cells expressing FRs and RFC, PC43-10, and Chinese hamster ovary (CHO) expressing RFC, and RT16 Chinese hamster ovary cells expressing FRs but no RFC. FIGS. 5, 6 and 7 show the IC₅₀ of each of the Sample compounds of the present invention towards each cancer cell line. The IC₅₀ is the inhibitory concentration required to effectuate fifty percent inhibition of cell growth.

Following the inventor's initial studies of antifolates MTX, PMX, RTX, LMTX GW1843U89 (Glaxo Wellcome) and PT523 to identify FR targeted agents with low level transport by human reduced folate carrier (hRFC) (Deng et al., 2008), we tested compounds of the present invention, namely, example compounds 1G104-G109, G114-G123, and G150, G152, G154 and G155 in KB human tumor cells (see FIGS. 5, 6, and 7). The compounds were initially tested for their growth inhibitory effects against KB human tumor cells which express FR alpha and hRFC but insignificant levels human proton coupled folate transporter (hPCFT), using a fluorescence-based (“Cell Titer-blue”) cytotoxicity scree. In KB cells, IC50s of 0.68, 0.14, 0.13, and 0.31 nM were measured from compounds G107, G117, G118, and G150. These unexpected results show that the compounds of the present invention have from a 10 to 20 fold increase, and in some cases a 100 fold increase, in the IC50s of currently used compounds in clinical practice, such as MTX, RTX, PMX, and LMTX. FR-targeted activity was confirmed by-co-treatments with folic acid (200 nM) which completely reversed growth inhibition of these agents. the compounds were also tested in isogenic Chinese hamster ovary (CHO) sublines, engineered to express human FR-(RT16) or hRFC (PC43-10). For PC43-10, results were compared to those for hRFC- and FR-null R2CHO cells from which they were derived, where as those for RT16 cells were compared to those for a parallel incubation in the presence of an elevated concentration of folic acid, as with the KB cells. The data shown in FIGS. 5-7 are mean values from 2-10 experiments (plus/minus SEM in parenthesis). Results are presented as IC50 values corresponding to the concentration that inhibit growth by 50% relative to cells incubated without drug (antifolate compound). Growth inhibition assays were performed for CHO sublines engineered to express hRFC (PC43-10), FR(RT16, D4), or hPCFT (R2/hPCFT4), for comparison with transporter null [R2, R2(VC)] CHO cells, and for the KB and IGROV1 (ovary) tumor sublines (expressing hRFC, FRR, and hPCFT). For the FR experiments, growth inhibition assays were performed in the presence and absence of 200 nM folic acid (FA).

### FIG. 5

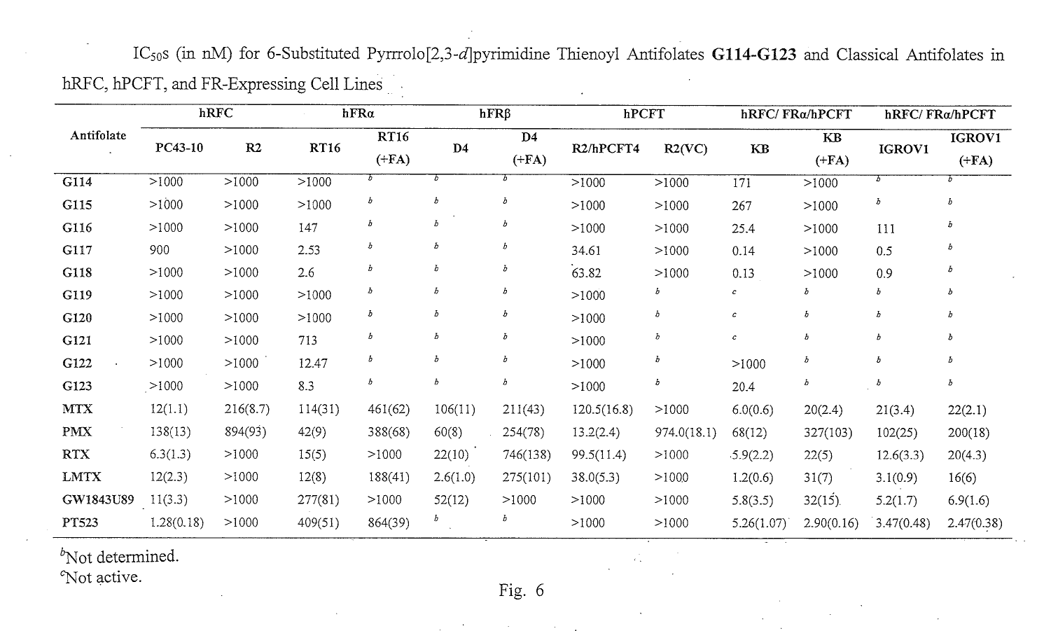

FIG. 5 shows that compounds G104-G109 (except G108) are selectively transported and inhibit cells (RT16) containing human FRα (folate receptor α) as the only transporter of folate/antifolates. Values range from 1.25-215 nM and compounds G105, G106 and G107 are much better inhibitors than the known clinical compounds MTX, PMX, etc. Compound G107 is the most potent analog evaluated in KB tumor cells and is 100-fold more potent than PMX (pemetrexed). In addition, except for LMTX, both G105 and G107 are better against KB tumor cells than MTX, PMX, RTX, GW1843U89 and PT523. These compounds are not transported by PC43-10 cells that contain only RFC and hence G104-G109 (except G108) are expected to selectively inhibit tumor cells that express folate receptor α and β. FIG. 6

FIG. 6 shows that compounds G114-G123 are not active against PC43-10 (RFC) containing folate/antifolate transporter but G116-G118 and G122-G123 are significantly potent against cells containing FRα (RT16) and G117 and G118 are also potent against PCFT expressing cells. Thus G116-G118 and G122-G123 are selective against tumor expressing folate/antifolate transporters. The standard compounds MTX, PMX, RTX are active against cells expressing hRFC (PC43-10 cells) hence are not selective. Compounds G116-G118 and particularly G117 and G118 are significantly more potent against KB tumor cells and ovarian cancer cells (IGROV1) compared to standards. Hence, G117-G118 will be nontoxic to normal cells expressing RFC and MTX, PMX and RTX do not possess this selectivity.

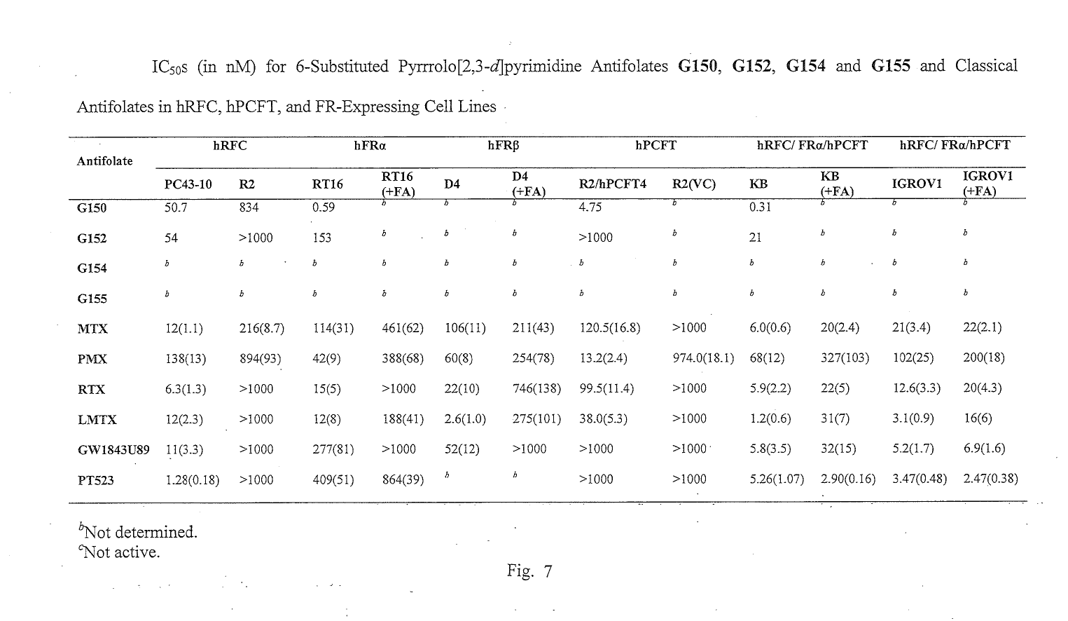

### FIG. 7

FIG. 7 shows that compound G150 is a potent inhibitor of KB cells much more than the standard compounds MTX, PMX, RTX etc. It is also about 100-fold more selective for hFRα (RT16) than hRFC (PC43-10). The compound also has transport via PCFT.

Preliminary (one-week) in-vivo studies evaluation of the compounds of the present invention in mice support that the compounds of the present invention suppress tumor growth and that the compounds of the present invention have an inhibitory effect on tumor growth compared to control (no compound administration). Thus, significant antitumor activity was detected after the first week of administration of the compounds of the present invention to mice.

The development of novel small molecule cytotoxins such as the compounds of Formulae I through X of the present invention that are selectively transported by hPCFT provide exciting new therapeutic applications for solid tumor targeting. This is based on the notion of effectively “highjacking” an essential biological characteristic of solid tumors, namely their acidic microenvironment, for selective delivery of the cytotoxic compounds of the present invention.

### Synthesis of Compounds

### Chemistry

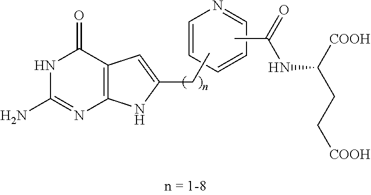

### 1. The synthesis of 2-amino-4-oxo-pyrrolo[2,3-d]pyrimidines with a pyridoyl side chain (4 carbon bridge)

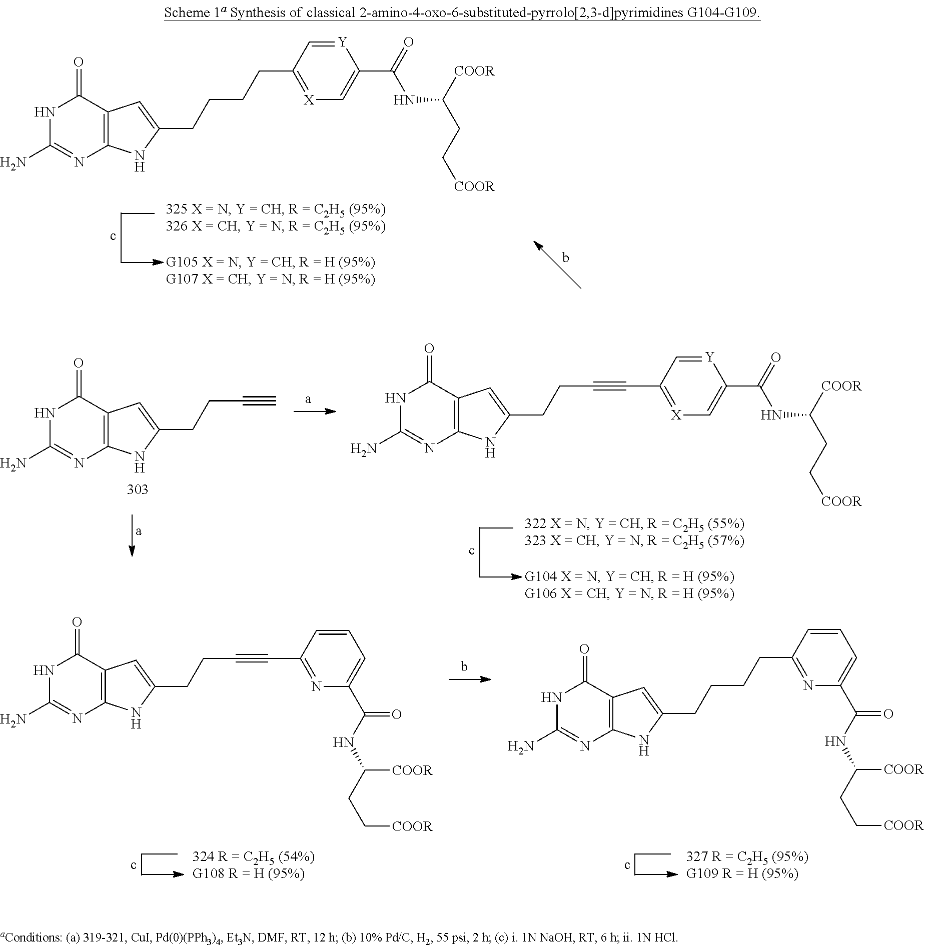

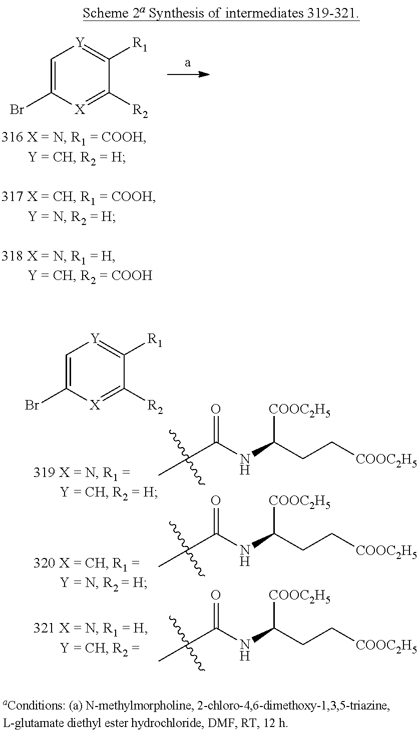

The synthesis of target compounds G104-G109 (Scheme 1) started from the reported intermediate 303¹. A Sonogashira coupling of 303 with 319-321 afforded 322-324 in 54-57% yield. Subsequent hydrogenation and saponification of 322-324 afforded target compounds G105, G107 and G109, respectively. Direct hydrolysis of intermediate 322-324 provided G104, G106 and G108 in 95% yield. Compounds 319-321 (Scheme 2) were synthesized by a peptide coupling of the commercially available bromo-substituted pyrido-carboxylic acids 316-318 with L-glutamate diethyl ester hydrochloride.

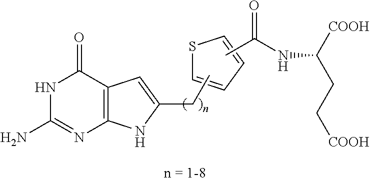

### 2. The synthesis of 2-amino-4-oxo-pyrrolo[2,3-d]pyrimidines with a thienoyl side chain (4 carbon bridge)

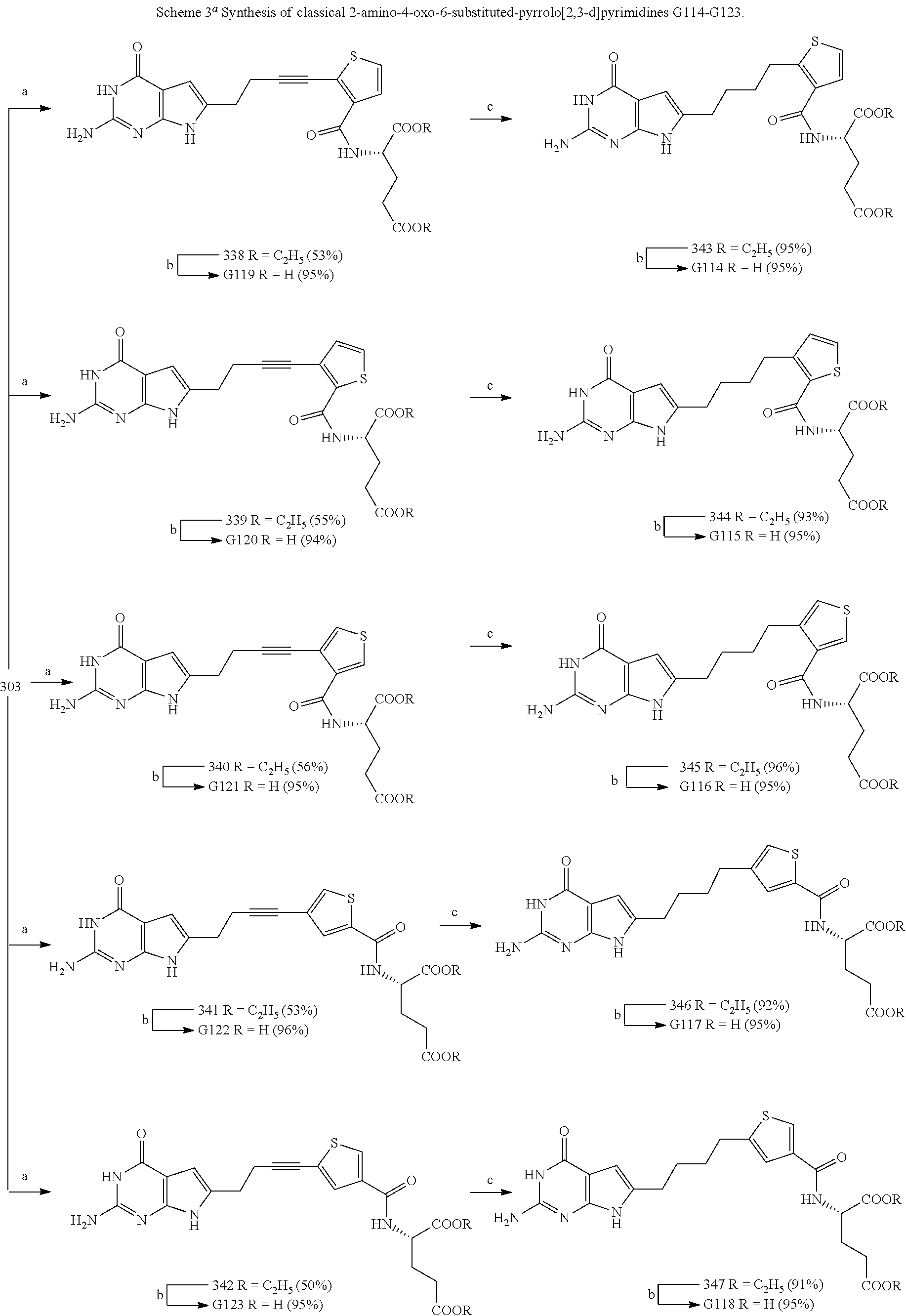

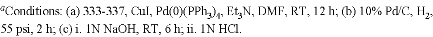

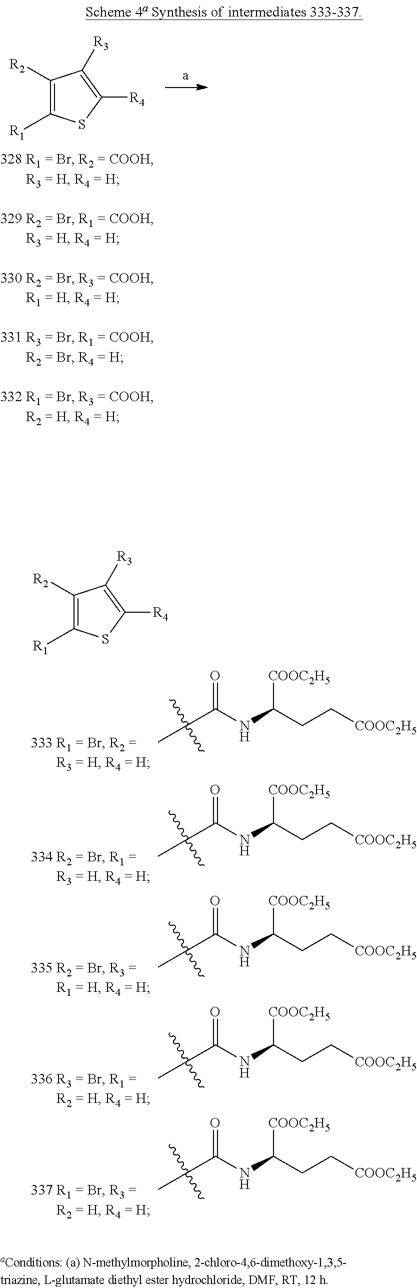

The synthesis of target compounds G114-G123 (Scheme 3) started from the reported intermediate 303¹, A Sonogashira coupling of 303 with 333-337 afforded 338-342 in 50-56% yield. Subsequent hydrogenation and saponification of 338-342 afforded target compounds G114-G118. Direct hydrolysis of intermediate 338-342 provided G119-G123 in 95% yield. Compounds 333-337 (Scheme 4) were synthesized by a peptide coupling of the commercially available bromo-substituted thiophene-carboxylic acids 328-332 with L-glutamate diethyl ester hydrochloride.

### 3. The synthesis of 2-amino-4-oxo-pyrrolo[2,3-d]pyrimidines with a thienoyl side chain (3 carbon bridge)

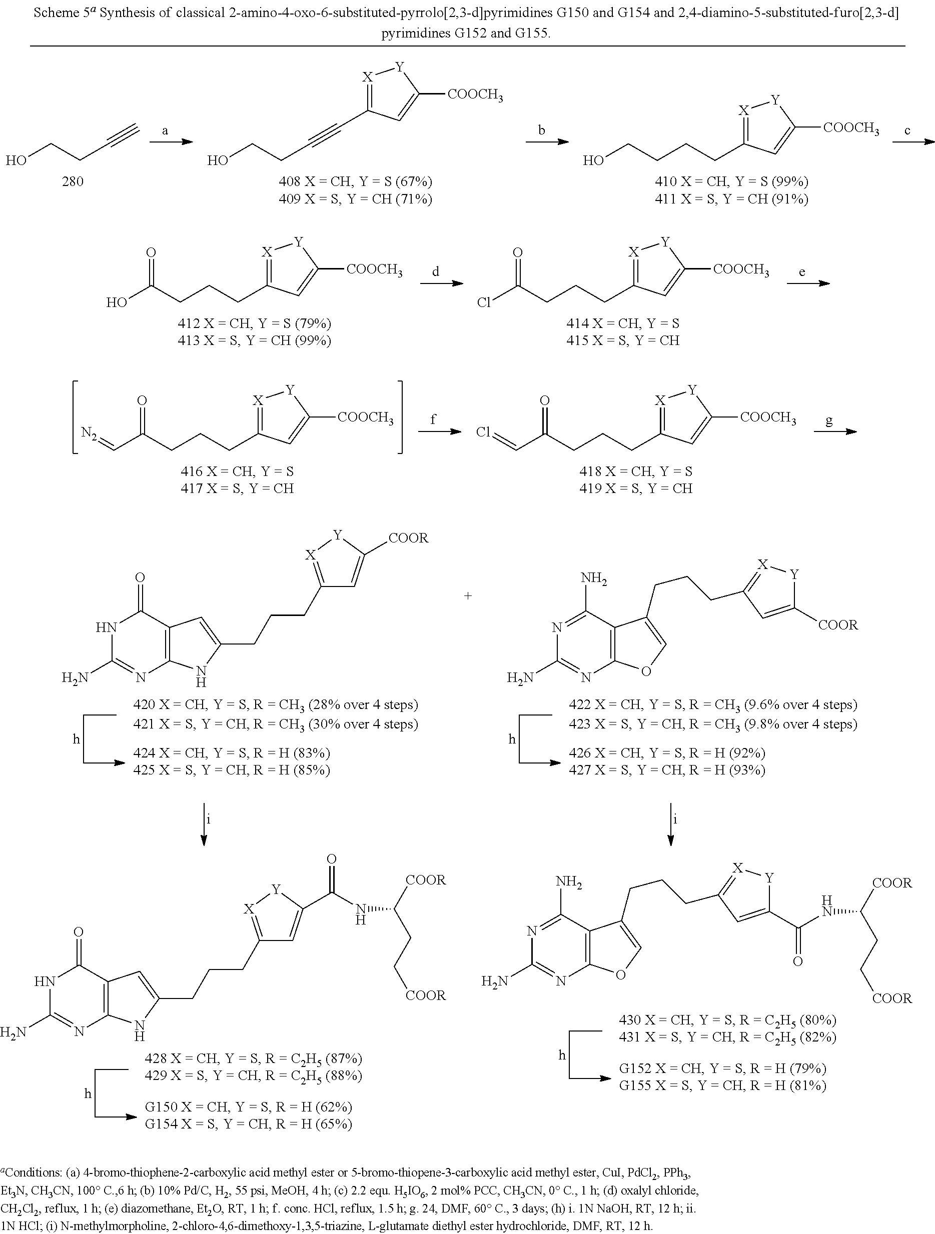

The synthesis of the target compounds was shown in Scheme 5. A palladium-catalyzed Sonogashira coupling of 4-bromo-thiophene-2-carboxylic acid methyl ester or 5-bromo-thiophene-3-carboxylic acid methyl ester with but-3-yn-1-ol, 280, afforded thiophenebutynyl alcohol 408-409, which was catalytically hydrogenated to give the saturated alcohol 410-411. Subsequent oxidation of 410-411 using perioidic acid and PCC as the oxidant afforded the carboxylic acid 412-413, which was converted to the acid chloride 414-415 and immediately reacted with diazomethane followed by cone. HCl to give the desired α-chloromethylketones 418-419. Condensation of 2,6-diamino-3H-pyrimidin-4-one, 24, with 418-419 at 60° C. for 3 days afforded 2-amino-4-oxo-pyrrolo[2,3-d]pyrimidines 420-421 and 2,4-diamino-furo[2,3-d]pyrimidines 422-423, respectively. Hydrolysis of 420-421 and 422-423 afforded the corresponding free acids 424-425 and 426-427. Subsequent coupling with L-glutamate diethyl ester using 2-chloro-4,6-dimethoxy-1,3,5-triazine as the activating agent afforded the diesters 428-429 and 430-431. Final saponification of the diesters gave the target compounds G150, G152, G154 and G155.

### Experimental

All evaporations were carried out in vacuum with a rotary evaporator. Analytical samples were dried in vacuo (0.2 mmHg) in a CHEM-DRY drying apparatus over P₂O₅ at 60° C. Melting points were determined on a MEL-TEMP II melting point apparatus with FLUKE 51 K/J electronic thermometer and are uncorrected. Nuclear magnetic resonance spectra for proton (¹H NMR) were recorded on Bruker Avance II 400 (400 MHz) and 500 (500 MHz) spectrometer. The chemical shift values are expressed in ppm (parts per million) relative to tetramethylsilane as an internal standard: s, singlet; d, doublet; t, triplet; q, quartet; m, multiplet; br, broad singlet. Thin-layer chromatography (TLC) was performed on Whatman Sil G/UV254 silica gel plates with a fluorescent indicator, and the spots were visualized under 254 and 366 nm illumination. Proportions of solvents used for TLC are by volume. Column chromatography was performed on a 230-400 mesh silica gel (Fisher, Somerville, N.J.) column. The amount (weight) of silica gel for column chromatography was in the range of 50-100 times the amount (weight) of the crude compounds being separated. Columns were dry-packed unless specified otherwise. Elemental analyses were performed by Atlantic Microlab, Inc., Norcross, Ga. Element compositions are within ±0.4% of the calculated values. Fractional moles of water or organic solvents frequently found in some analytical samples of antifolates could not be prevented despite 24-48 h of drying in vacuo and were confirmed where possible by their presence in the ¹H NMR spectra. High-resolution mass spectrometry (HRMS) was performed on a Waters Q-TOF (API-US) by Department of Chemistry, University of Pittsburgh, Pittsburgh, Pa. All solvents and chemicals were purchased from Aldrich Chemical Co. and Fisher Scientific and were used as received.

### General Procedure for the Synthesis of Compounds 322-324.

To a 250-mL round-bottomed flask, equipped with a magnetic stirrer and gas inlet, was added a mixture of tetrakis(triphenylphosphine)palladium(0) (370 mg, 0.32 mmol), triethylamine (2.02 g, 20 mmol), 319, 320 or 321 (1.13 g, 3 mmol) and anhydrous DMF (20 mL). To the stirred mixture, under N₂, were added copper(I) iodide (61 mg, 0.32 mmol) and 303 (404 mg, 2 mmol). The reaction mixture was then stirred at room temperature overnight (17-18 h). To the reaction maturate was added silica gel (1 g), and the solvent was evaporated reduced pressure. The resulting plug was loaded on to a silica gel column (2×12 cm) and eluted with CHCl₃ followed by 3% MeOH in CHCl₃ and then 5% MeOH in CHCl₃. Fractions with desired R_{f} (TLC) were pooled and evaporated to afford 322-324.

(S)-2-({6-[4-(2-Amino-4-oxo-4,7-dihydro-3H-pyrrolo[2,3-d]pyrimidin-6-yl)-but-1-ynyl]-pyridine-3-carbonyl}-amino)-pentanedioic acid diethyl ester (322): Compound 322 was prepared using the general method described for the preparation of 322, from 303 (404 mg, 2 mmol) and (S)-2-[(6-bromo-pyridine-3-carbonyl)-amino]-pentanedioic acid diethyl ester, 319 (1.16 g, 3 mmol) to give 559 mg (55%) of 322 as a brown powder. mp 81-82° C.; TLC R_{f} 0.53 (CHCl₃/MeOH 5:1); ¹H NMR (DMSO-d₆): δ 1.15-1.18 (m, 6H, COOCH₂CH₃), 1.97-2.10 (m, 2H, β-CH₂), 2.42-2.46 (m, 2H, γ-CH₂), 2.80 (m, 4H, CH₂CH₂), 4.01-4.13 (m, 4H, COOCH₂CH₃), 4.44 (m, 1H, α-CH), 6.01 (s, 3H, C5-CH, 2-NH₂, exch), 7.53-7.55 (d, J=8.0 Hz, 1H, Ar), 8.16-8.18 (d, J=8.0 Hz, 1H, CONH, exch), 8.93-8.97 (m, 2H, Ar), 10.16 (s, 1H, 3-NH, exch), 10.90 (s, 1H, 7-NH, exch).

(S)-2-({5-[4-(2-Amino-4-oxo-4,7-dihydro-3H-pyrrolo[2,3-d]pyrimidin-6-yl)-but-1-ynyl]-pyridine-2-carbonyl}-amino)-pentanedioic acid diethyl ester (323): Compound 323 was prepared using the general method described for the preparation of 322-324, from 303 (404 mg, 2 mmol) and (S)-2-[(5-bromo-pyridine-2-carbonyl)-amino]-pentanedioic acid diethyl ester, 320 (1.16 g, 3 mmol) to give 579 mg (57%) of 323 as a brown powder, mp 79-80° C.; TLC R_{f} 0.53 (CHCl₃/MeOH 5:1); ¹H NMR (DMSO-d₆): δ 1.12-1.18 (m, 6H, COOCH₂CH₃), 2.03-2.17 (m, 2H, β-CH₂), 2.33-2.38 (m, 2H, γ-CH₂), 2.79 (m, 4H, CH₂CH₂), 3.97-4.13 (m, 4H, COOCH₂CH₃), 4.46-4.52 (m, 1H, α-CH), 6.01 (s, 3H, C5-CH, 2-NH₂, exch), 7.96 (s, 2H, Ar), 8.62 (s, 1H, Ar), 8.98-9.00 (d, J=7.6 Hz, 1H, CONH, exch), 10.15 (s, 1H, 3-NH, exch), 10.89 (s, 1H, 7-NH, exch).

(S)-2-({6-[4-(2-Amino-4-oxo-4,7-dihydro-3H-pyrrolo[2,3-d]pyrimidin-6-yl)-but-1-ynyl]-pyridine-2-carbonyl}-amino)-pentanedioic acid diethyl ester (324): Compound 24 was prepared using the general method described for the preparation of 322-324, from 303 (404 mg, 2 mmol) and (S)-2-[(6-bromo-pyridine-2-carbonyl)-amino]-pentanedioic acid diethyl ester, 321 (1.16 g, 3 mmol) to give 548 mg (54%) of 324 as a brown powder. mp 80-81° C.; TLC R_{f} 0.52 (CHCl₃/MeOH 5:1); ¹H NMR (DMSO-d₆): δ 1.14-1.21 (m, 6H, COOCH₂CH₃), 2.07-2.22 (m, 2H, β-CH₂), 2.36-2.39 (t, J=8.0 Hz, 2H, γ-CH₂), 2.80-2.85 (m, 4H, CH₂CH₂), 4.00-4.05 (q, J=7.0 Hz, 2H, COOCH₂CH₃), 4.10-4.16 (m, 2H, COOCH₂CH₃), 4.52-4.56 (m, 1H, α-CH), 6.01 (s, 2H, 2-NH₂, exch), 6.04 (s, 1H, C5-CH), 7.65-7.67 (m, 1H, Ar), 7.95-8.0 (m, 2H, Ar), 8.83-8.85 (d, J=8.0 Hz, 1H, CONH, exch), 10.16 (s, 1H, 3-NH, exch), 10.91 (s, 1H, 7-NH, exch).

### General Procedure for the Synthesis of Compounds 325-327.

To a Parr flask was added 322, 323 or 324 (200 mg, 0.39 mmol), 10% palladium on activated carbon (100 mg), and MeOH (50 mL). Hydrogenation was carried out at 55 psi of H₂ for 4 h. The reaction mixture was filtered through Celite, washed with MeOH (100 mL) and concentrated under reduced pressure to give 325-327.

(S)-2-({6-[4-(2-Amino-4-oxo-4,7-dihydro-3H-pyrrolo[2,3-d]pyrimidin-6-yl)-butyl]-pyridine-3-carbonyl}-amino)-pentanedioic acid diethyl ester 325): Compound 325 was prepared using the general method described for the preparation of 325-327, from 322 (200 mg, 0.40 mmol) to give G115 mg (95%) of 325 as a light yellow powder. mp 81-82° C.; TLC R_{f} 0.54 (CHCl₃/MeOH 5:1); ¹H NMR (DMSO-d₆): δ 1.17-1.20 (m, 6H, COOCH₂CH₃), 1.60 (m, 2H, CH₂), 1.71 (m, 2H, CH₂), 2.01-2.11 (m, β-CH₂), 2.44-2.47 (m, 2H, γ-CH₂), 2.53 (m, 2H, CH₂), 2.86-2.89 (m, 2H, CH₂), 4.03-4.13 (m, 4H, COOCH₂CH₃), 4.46 (m, 1H, α-CH), 5.91 (s, 1H, C5-CH), 6.46 (s, 2H, 2-NH₂, exch), 7.55 (d, J=8.0 Hz, 1H, Ar), 8.31 (d, J=8.0 Hz, 1H, CONH, exch), 8.99 (m, 2H, Ar), 10.57 (s, 1H, 3-NH, exch), 11.07 (s, 1H, 7-NH, exch).

(S)-2-({5-[4-(2-Amino-4-oxo-4,7-dihydro-3H-pyrrolo[2,3-d]pyrimidin-6-yl)-butyl]-pyridine-2-carbonyl}-amino)-pentanedioic acid diethyl ester (326): Compound 326 was prepared using the general method described for the preparation of 325-327, from 323 (200 mg, 0.40 mmol) to give G115 mg (95%) of 326 as a light yellow powder. mp 77-78° C.; TLC R_{f} 0.53 (CHCl₃/MeOH 5:1); ¹H NMR (DMSO-d₆): δ 1.10-1.19 (m, 6H, COOCH₂CH₃), 1.60 (m, 4H, CH₂CH₂), 2.03-2.17 (m, 2H, β-CH₂), 2.34-2.38 (t, J=8.0 Hz, 2H, γ-CH₂), 2.52-2.54 (m, 2H, CH₂), 2.69-2.72 (m, 2H, CH₂), 3.97-4.13 (m, 4H, COOCH₂CH₃), 4.48-4.53 (m, 1H, α-CH), 5.92 (s, 1H, C5-CH), 6.53 (s, 2H, 2-NH₂, exch), 7.81-7.83 (dd, J₁=2.0 Hz, J₁=8.0 Hz, 1H, Ar), 7.92-7.94 (d, J=8.0 Hz, 1H, Ar), 8.51 (s, 1H, Ar), 8.88-8.90 (d, J=8.0 Hz, 1H, CONH, exch), 10.61 (s, 1H, 3-NH, exch), 11.09 (s, 1H, 7-NH, exch).

(S)-2-({6-[4-(2-Amino-4-oxo-4,7-dihydro-3H-pyrrolo[2,3-d]pyrimidin-6-yl)-butyl]-pyridine-2-carbonyl}-amino)-pentanedioic acid diethyl ester (327): Compound 327 was prepared using the general method described for the preparation of 325-327, from 324 (200 mg, 0.40 mmol) to give G115 mg (95%) of 327 as a light yellow powder. mp 80-81° C.; TLC R_{f} 0.54 (CHCl₃/MeOH 5:1); ¹H NMR (DMSO-d₆): δ 1.11-1.14 (m, 6H, COOCH₂CH₃), 1.62-1.79 (m, 4H, CH₂CH₂), 2.05-2.22 (m, 2H, β-CH₂), 2.37-2.40 (t, J=8.0 Hz, 2H, γ-CH₂), 2.53-2.56 (t, J=7.5 Hz, 2H, CH₂), 2.84-2.87 (t, J=7.5 Hz, 2H, CH₂), 3.99-4.03 (q, J=7.0 Hz, 2H, COOCH₂CH₃), 4.10-4.17 (m, 2H, COOCH₂CH₃), 4.52-4.57 (m, 1H, α-CH), 5.87 (s, 1H, C5-CH), 5.99 (s, 2H, 2-NH₂, exch), 7.49-7.50 (d, J=7.5 Hz, 1H, Ar), 8.83-7.92 (m, 2H, Ar), 8.77-8.79 (d, J=8.0 Hz, 1H, CONH, exch), 10.14 (s, 1H, 3-NH, exch), 10.81 (s, 1H, 7-NH, exch).

### General Procedure for the Synthesis of Compounds G104-G109.

To a solution of 322-327 (100 mg, 0.19 mmol) in MeOH (10 mL) was added 1 N NaOH (5 mL) and the mixture was stirred under N₂ at room temperature for 16 h. TLC showed the disappearance of the starting material (R_{f} 0.54, CHCl₃/MeOH 5:1) and one major spot at the origin. The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford G104-G109,

(S)-2-({6-[4-(2-Amino-4-oxo-4,7-dihydro-3H-pyrrolo[2,3-d]pyrimidin-6-yl)-butyl]-pyridine-3-carbonyl}-amino)-pentanedioic acid (G105): Compound G105 was prepared using the general method described for the preparation of G104-G109, from 325 (100 mg, 0.19 mmol) to give 82 mg (95%) of G105 as a light yellow powder. mp 177-178° C.; ¹H NMR (DMSO-d₆): δ 1.54-1.71 (m, 4H, CH₂CH₂), 1.90-2.12 (m, 2H, β-CH₂), 2.33-2.37 (t, J=7.6 Hz, 2H, γ-CH₂), 2.51 (m, 2H, CH₂), 2.77-2.81 (m, 2H, CH₂), 4.37-4.42 (m, 1H, α-CH), 5.83 (s, 1H, C5-CH), 5.95 (s, 2H, 2-NH₂, exch), 7.34-7.36 (d, J=8.4 Hz, 1H, Ar), 8.10-8.12 (d, J=8.4 Hz, 1H, Ar), 8.72-8.74 (d, J=7.2 Hz, 1H, CONH, exch), 8.91 (s, 1H, Ar), 10.11 (s, 1H, 3-NH, exch), 10.78 (s, 1H, 7-NH, exch), 12.44 (br, 2H, COOH, exch). Anal, calcd for (C₂₁H₂₄N₆O₆.0.7H₂O): C, 53.77; H, 5.46; N, 17.92; Found: C, 53.81; H, 5.50; N, 17.82.

(S)-2-({5-[4-(2-Amino-4-oxo-4,7-dihydro-3H-pyrrolo[2,3-d]pyrimidin-6-yl)-butyl]-pyridine-2-carbonyl}-amino)-pentanedioic acid (G107): Compound G107 was prepared using the general method described for the preparation of G104-G109, from 326 (100 mg, 0.19 mmol) to give 82 mg (95%) of G107 as a light yellow powder. mp 178-179° C.; ¹H NMR (DMSO-d₆): δ 1.56 (m, 4H, CH₂CH₂), 1.97-2.14 (m, 2H, β-CH₂), 2.25-2.29 (t, J=7.6 Hz, 2H, γ-CH₂), 2.51 (m, 2H, CH₂), 2.68-2.71 (m, 2H, CH₂), 4.41-4.46 (m, 1H, α-CH), 5.85 (s, 1H, C5-CH), 5.95 (s, 2H, 2-NH₂, exch), 7.34-7.36 (d, J=8.4 Hz, 1H, Ar), 7.80-7.82 (m, 1H, Ar), 8.49 (s, 1H, Ar), 8.75-8.77 (d, J=8.0 Hz, 1H, CONH, exch), 10.12 (s, 1H, 3-NH, exch), 10.78 (s, 1H, 7-NH, exch), 12.59 (br, 2H, COOH, exch). Anal. calcd for (C₂₁H₂₄N₆O₆.1.2H₂O): C, 52.76; H, 5.57; N, 17.58; Found: C, 52.90; H, 5.43; N, 17.43.

(S)-2-({6-[4-(2-Amino-4-oxo-4,7-dihydro-3H-pyrrolo[2,3-d]pyrimidin-6-yl)-butyl]-pyridine-2-carbonyl}-amino)-pentanedioic acid (G109): Compound G109 was prepared using the general method described for the preparation of G104-G109, from 327 (100 mg, 0.19 mmol) to give 82 mg (95%) of G109 as a light yellow powder. mp 175-176° C.; ¹H NMR (DMSO-d₆): δ 1.62-1.79 (m, 4H, CH₂CH₂), 1.99-2.20 (m, 2H, β-CH₂), 2.29-2.32 (t, J=7.5 Hz, 2H, γ-CH₂), 2.53-2.56 (t, J=12.0 Hz, 2H, CH₂), 2.84-2.87 (t, J=7.5 Hz, 2H, CH₂), 4.48-4.53 (m, 1H, α-CH), 5.88 (s, 1H, C5-CH), 5.96 (s, 2H, 2-NH₂, exch), 7.48-7.50 (dd, Jᵢ=1.0 Hz, J₂=7.5 Hz, 1H, Ar), 7.84-7.92 (m, 2H, Ar), 8.71-8.73 (d, J=8.5 Hz, 1H, CONH, exch), 10.14 (s, 1H, 3-NH, exch), 10.80 (s, 1H, 7-NH, exch), 12.51 (br, 2H, COOH, exch), Anal. calcd for (C₂₁H₂₄N₆O₆. 1.0 H₂O): C, 53.16; H, 5.52; N, 17.71; Found: C, 53.14; H, 5.46; N, 17.65.

(S)-2-({6-[4-(2-Amino-4-oxo-4,7-dihydro-3H-pyrrolo[2,3-d]pyrimidin-6-yl)-but-1-ynyl]-pyridine-3-carbonyl}-amino)-pentanedioic acid (G104): Compound G104 was prepared using the general method described for the preparation of G104-G109, from 322 (100 mg, 0.20 mmol) to give 84 mg (95%) of G104 as a light yellow powder. mp 178-179° C.; ¹H NMR (DMSO-d₆): δ 1.94-2.07 (m, 2H, β-CH₂), 2.33-2.37 (m, 2H, γ-CH₂), 2.66 (m, 2H, CH₂), 2.94 (m, 2H, CH₂), 4.40 (m, 1H, α-CH), 6.00 (s, 3H, C5-CH, 2-NH₂, exch), 7.52-7.54 (d, J=8.0 Hz, 1H, Ar), 8.16-8.19 (d, J=8.0 Hz, 1H, CONH, exch), 8.94-9.02 (m, 2H, Ar), 10.16 (s, 1H, 3-NH, exch), 10.89 (s, 1H, 7-NH, exch), 12.55 (br, 2H, COOH, exch). Anal. calcd for (C₂₁H₂₀N₆O₆.0.8H₂O): C, 54.03; H, 4.66; N, 18.00; Found: C, 53.99; H, 4.60; N, 18.10.

(S)-2-({5-[4-(2-Amino-4-oxo-4,7-dihydro-3H-pyrrolo[2,3-d]pyrimidin-6-yl)-but-1-ynyl]-pyridine-2-carbonyl}-amino)-pentanedioic acid (G106): Compound G106 was prepared using the general method described for the preparation of G104-G109, from 323 (100 mg, 0.20 mmol) to give 84 mg (95%) of G106 as a light yellow powder. mp 181-182° C.; ¹H NMR (DMSO-d₆): δ 2.02-2.11 (m, 2H, β-CH₂), 2.27 (m, 2H, γ-CH₂), 2.80 (m, 4H, CH₂CH₂), 4.44 (m, 1H, α-CH), 6.01 (s, 3H, C5-CH, 2-NH₂, exch), 7.97 (s, 2H, Ar), 8.62 (s, 1H, Ar), 8.84 (s, 1H, CONH, exch), 10.16 (s, 1H, 3-NH, exch), 10.89 (s, 1H, 7-NH, exch), 12.57 (br, 2H, COOH, exch). Anal. calcd for (C₂₁H₂₀N₆O₆. 0.2 CHCl₃): C, 53.46; H, 4.27; N, 17.64; Found: C, 53.62; H, 4.60; N, 17.29.

(S)-2-({6-[4-(2-Amino-4-oxo-4,7-dihydro-3H-pyrrolo[2,3-d]pyrimidin-6-yl)-but-1-ynyl]-pyridine-2-carbonyl}-amino)-pentanedioic acid (G108); Compound G108 was prepared using the general method described for the preparation of G104-G109, from 324 (100 mg, 0.20 mmol) to give 84 mg (95%) of G108 as a light yellow powder. mp 180-181° C.; ¹H NMR (DMSO-d₆): δ 1.94-2.20 (m, 2H, β-CH₂), 2.28-2.31 (t, J=7.5 Hz, 2H, γ-CH₂), 2.79-2.86 (m, 4H, CH₂CH₂), 4.40 (m, 1H, α-CH), 6.00 (s, 1H, C5-CH), 6.04 (s, 2H, 2-NH₂, exch), 7.64-7.66 (dd, J₁=2.5 Hz, J₂=8.5 Hz, 1H, Ar), 7.96-8.00 (m, 2H, Ar), 8.70-8.21 (d, J=8.5 Hz, 1H, CONH, exch), 10.17 (s, 1H, 7-NH, exch), 10.91 (s, 1H, 7-NH, exch), 12.53 (br, 2H, COOH, exch). Anal. calcd for (C₂₁H₂₀N₆O₆.1.5H₂O): C, 52.61; H, 4.84; N, 17.53; Found: C, 52.50; H, 4.79; N, 17.43.

### General Procedure for the Synthesis of Compounds 338-342.

To a 250-mL round-bottomed flask, equipped with a magnetic stirrer and gas inlet, was added a mixture of tetrakis(triphenylphosphine)palladium(0) (277 mg, 0.24 mmol), triethylamine (1.52 g, 15 mmol), 333-337 (882 mg, 2.25 mmol) and anhydrous DMF (20 mL). To the stirred mixture, under N₂, was added copper(I) iodide (46 mg, 0.24 mmol) and 303 (303 mg, 1.5 mmol), and the reaction mixture was stirred at room temperature overnight (17-18 h). Silica gel (0.5 g) was then added, and the solvent was evaporated under reduced pressure. The resulting plug was loaded on to a silica gel column (1.5×12 cm) and eluted with CHCl₃ followed by 3% MeOH in CHCl₃ and then 5% MeOH in CHCl₃. Fractions with desired R_{f} (TLC) were pooled and evaporated to afford 338-342.

(S)-2-({2-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid diethyl ester (338): Compound 338 was prepared using the general method described for the preparation of 338-342, from 303 (303 mg, 1.5 mmol) and (S)-2-[(2-bromo-thiophene-3-carbonyl)-amino]-pentanedioic acid diethyl ester, 333 (882 mg, 2.25 mmol) to give 412 mg (53%) of 338 as a brown powder. mp 81-82° C.; TLC R_{f} 0.53 (CHCl₃/MeOH 5:1); ¹H NMR (DMSO-d₆): δ 1.16-1.22 (m, 6H, COOCH₂CH₃), 1.92-2.14 (m, 2H, β-CH₂), 2.43-2.46 (t, J=8 Hz, 2H, γ-CH₂), 2.80 (m, 4H, CH₂CH₂), 4.01-4.52 (q, J=7 Hz, 2H, COOCH₂CH₃), 4.10-4.16 (m, 2H, COOCH₂CH₃), 4.43-4.48 (m, 1H, α-CH), 5.99 (s, 1H, C5-CH), 6.01 (s, 2H, 2-NH₂, exch), 7.35-7.36 (d, J=5.5 Hz, 1H, Ar), 7.53-7.54 (d, J=5.5 Hz, 1H, Ar), 8.34-8.36 (d, J=7.5 Hz, 1H, CONH, exch), 10.16 (s, 1H, 3-NH, exch), 10.88 (s, 1H, 7-NH, exch).

(S)-2-({3-[4-(2-Amino-4-oxo-4,7-dihydro-3H-pyrrolo[2,3-d]pyrimidin-6-yl)-but-1-ynyl]-thiophene-2-carbonyl}-amino)-pentanedioic acid diethyl ester (339): Compound 339 was prepared using the general method described for the preparation of 338-342, from 303 (303 mg, 1.5 mmol) and (S)-2-[(3-bromo-thiophene-2-carbonyl)-amino]-pentanedioic acid diethyl ester, 334 (882 mg, 2.25 mmol) to give 423 mg (55%) of 339 as a brown powder. mp 77-78° C.; TLC R_{f} 0.52 (CHCl₃/MeOH 5:1); ¹H NMR (DMSO-d₆): δ 1.16-1.22 (m, 6H, COOCH₂CH₃), 1.92-2.18 (m, 2H, β-CH₂), 2.39-2.43 (m, 2H, γ-CH₂), 2.84 (m, 4H, CH₂CH₂), 3.99-4.03 (q, J=7 Hz, 2H, COOCH₂CH₃), 4.14-4.18 (q, J=7 Hz, 2H, COOCH₂CH₃), 4.53-4.57 (m, 1H, α-CH), 6.00 (s, 1H, C5-CH), 6.01 (s, 2H, 2-NH₂, exch), 7.16-7.17 (d, J=5.0 Hz, 1H, Ar), 7.81-7.82 (d, J=5.0 Hz, 1H, Ar), 8.19-8.20 (d, J=7.5 Hz, 1H, CONH, exch), 10.16 (s, 1H, 3-NH, exch), 10.91 (s, 1H, 7-NH, exch).

(S)-2-({4-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid diethyl ester (340): Compound 340 was prepared using the general method described for the preparation of 338-342, from 303 (303 mg, 1.5 mmol) and (S)-2-[(4-bromo-thiophene-3-carbonyl)-amino]-pentanedioic acid diethyl ester, 335 (882 mg, 2.25 mmol) to give 433 mg (56%) of 340 as a brown powder. mp 80-81° C.; TLC R_{f} 0.53 (CHCl₃/MeOH 5:1); ¹H NMR (DMSO-d₆): δ 1.16-1.22 (m, 6H, COOCH₂CH₃), 1.92-2.14 (m, 2H, β-CH₂), 2.42-2.46 (m, 2H, γ-CH₂), 2.69-2.78 (m, 4H, CH₂CH₂), 4.01-4.05 (q, J=7 Hz, 2H, COOCH₂CH₃), 4.11-4.16 (m, 2H, COOCH₂CH₃), 4.45-4.50 (m, 1H, α-CH), 5.99 (s, 1H, C5-CH), 6.00 (s, 2H, 2-NH₂, exch), 7,16-7.17 (d, J=3.5 Hz, 1H, Ar), 8.08-8.09 (d, J=3.5 Hz, 1H, Ar), 8.40-8.41 (d, J=7.5 Hz, 1H, CONH, exch), 10.16 (s, 1H, 3-NH, exch), 10.88 (s, 1H, 7-NH, exch),

(S)-2-({4-[4-(2-Amino-4-oxo-4,7-dihydro-3H-pyrrolo[2,3-d]pyrimidin-6-yl)-but-1-ynyl]-thiophene-2-carbonyl}-amino)-pentanedioic acid diethyl ester (341): Compound 341 was prepared using the general method described for the preparation of 338-342, from 303 (303 mg, 1.5 mmol) and (S)-2-[(4-bromo-thiophene-2-carbonyl)-amino]-pentanedioic acid diethyl ester, 336 (882 mg, 2.25 mmol) to give 406 mg (53%) of 341 as a brown powder. mp 79-80° C.; TLC R_{f} 0.53 (CHCl₃/MeOH 5:1); ¹H NMR (DMSO-d₆): δ 1.16-1.22 (m, 6H, COOCH₂CH₃), 1.92-2.12 (m, 2H, β-CH₂), 2.42-2.45 (t, J=7.5 Hz, 2H, γ-CH₂), 2.71-2.78 (m, 4H, CH₂CH₂), 4.03-4.07 (q, J=7 Hz, 2H, COOCH₂CH₃), 4.09-4.13 (q, J=6.5 Hz, 2H, COOCH₂CH₃), 4.36-4.40 (m, 1H, α-CH), 6.00 (s, 1H, C5-CH), 6.02 (s, 2H, 2-NH₂, exch), 7.87-7.88 (m, 2H, Ar), 8.76-8.77 (d, J=7.5 Hz, 1H, CONH, exch), 10.17 (s, 1H, 3-NH, exch), 10.90 (s, 1H, 7-NH, exch).

(S)-2-({5-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid diethyl ester (342): Compound 342 was prepared using the general method described for the preparation of 338-342, from 303 (303 mg, 1.5 mmol) and (S)-2-[(5-bromo-thiophene-3-carbonyl)-amino]-pentanedioic acid diethyl ester, 337 (882 mg, 2.25 mmol) to give 387 mg (50%) of 342 as a brown powder, mp 81-82° C.; TLC R_{f} 0.53 (CHCl₃/MeOH 5:1); NMR (DMSO-d₆): δ 1.16-1.22 (m, 6H, COOCH₂CH₃), 1.92-2.12 (m, 2H, β-CH₂), 2.41-2.44 (t, J=7.5 Hz, 2H, γ-CH₂), 2.78 (m, 4H, CH₂CH₂), 4.03-4.07 (q, J=7 Hz, 2H, COOCH₂CH₃), 4.09-4.13 (m, 2H, COOCH₂CH₃), 4.36-4.41 (m, 1H, α-CH), 6.00 (s, 1H, C5-CH), 6.01 (s, 2H, 2-NH₂, exch), 7.58-7.59 (d, J=1.5 Hz, 1H, Ar), 8.12-8.13 (d, J=1.5 Hz, 1H, Ar), 8.56-8.58 (d, J=7.5 Hz, 1H, CONH, exch), 10.16 (s, 1H, 3-NH, exch), 10.88 (s, 1H, 7-NH, exch).

### General Procedure for the Synthesis of Compounds 343-347.

To a Parr flask was added 338-342 (200 mg, 0.39 mmol), 10% palladium on activated carbon (100 mg), and MeOH (50 mL). Hydrogenation was carried out at 55 psi of H₂ for 4 h. The reaction mixture was filtered through Celite, washed with MeOH (100 mL) and concentrated under reduced pressure to give 343-347.

(S)-2-({2-[4-(2-Amino-4-oxo-4,7-dihydro-3H-pyrrolo[2,3-d]pyrimidin-6-yl)-butyl]-thiophene-3-carbonyl}-amino)-pentanedioic acid diethyl ester (343): Compound 343 was prepared using the general method described for the preparation of 343-347, from 338 (200 mg, 0.39 mmol) to give 191 mg (95%) of 343 as a light yellow powder. mp 81-82° C.; TLC R_{f} 0.54 (CHCl₃/MeOH 5:1); ¹H NMR (DMSO-d₆): δ 1.15-1.17 (m, 6H, COOCH₂CH₃), 1.61 (m, 4H, CH₂CH₂), 1.91-2.12 (m, 2H, β-CH₂), 2.41-2.44 (t, J=7.0 Hz, 2H, γ-CH₂), 2.51-2.53 (m, 4H, CH₂CH₂), 4.03-4.07 (q, J=7 Hz, 2H, COOCH₂CH₃), 4.08-4.13 (m, 2H, COOCH₂CH₃), 4.35-4.40 (m, 1H, α-CH), 5.96 (s, 3H, C5-CH, 2-NH₂, exch), 7.35 (s, 2H, Ar), 8.40-8.42 (d, J=7.5 Hz, 1H, CONH, exch), 10.99 (s, 1H, 3-NH, exch), 11.35 (s, 1H, 7-NH, exch).

(S)-2-({3-[4-(2-Amino-4-oxo-4,7-dihydro-3H-pyrrolo[2,3-d]pyrimidin-6-yl)-butyl]-thiophene-2-carbonyl}-amino)-pentanedioic acid diethyl ester (344): Compound 344 was prepared using the general method described for the preparation of 343-347, from 339 (200 mg, 0.39 mmol) to give 187 mg (93%) of 344 as a light yellow powder. mp 80-81° C.; TLC R_{f} 0.53 (CHCl₃/MeOH 5:1); ¹H NMR (DMSO-d₆): δ 1.15-1.20 (m, 6H, COOCH₂CH₃), 1.57 (m, 4H, CH₂CH₂), 1.93-2.12 (m, 2H, β-CH₂), 2.40-2.43 (t, J=8.0 Hz, 2H, γ-CH₂), 2.51 (m, 2H, CH₂), 2.87 (m, 2H, CH₂), 4.03-4.07 (q, J=7.0 Hz, 2H, COOCH₂CH₃), 4.08-4.14 (m, 2H, COOCH₂CH₃), 4.34-4.39 (m, 1H, α-CH), 5.96 (m, 3H, C5-CH, 2-NH₂, exch), 7.02-7.03 (d, J=5.0 Hz, 1H, Ar), 7.59-7.60 (d, J=5.0 Hz, 1H, Ar), 8.39-8.40 (d, J=7.5 Hz, 1H, CONH, exch), 11.09 (s, 1H, 3-NH, exch), 11.41 (s, 1H, 7-NH, exch).

(S)-2-({4-[4-(2-Amino-4-oxo-4,7-dihydro-3H-pyrrolo[2,3-d]pyrimidin-6-yl)-butyl]-thiophene-3-carbonyl}-amino)-pentanedioic acid diethyl ester (345): Compound 345 was prepared using the general method described for the preparation of 343-347, from 340 (200 mg, 0.39 mmol) to give 193 mg (96%) of 345 as a light yellow powder. mp 79-80° C.; TLC R_{f} 0.54 (CHCl₃/MeOH 5:1); ¹H NMR (DMSO-d₆): δ 1.17-1.18 (m, 6H, COOCH₂CH₃), 1.56 (m, 4H, CH₂CH₂), 1.91-2.11 (m, 2H, β-CH₂), 2.43-2.46 (t, J=7.5 Hz, 2H, γ-CH₂), 2.46-2.49 (m, 2H, CH₂), 2.75-2.77 (m, 2H, CH₂), 4.03-4.08 (q, J=7.0 Hz, 2H, COOCH₂CH₃), 4.08-4.13 (m, 2H, COOCH₂CH₃), 4.35-4.39 (m, 1H, α-CH), 5.83 (s, 1H, C5-CH), 5.98 (s, 2H, 2-NH₂, exch), 7.20-7.21 (d, J=3.5 Hz, 1H, Ar), 7.92-7.93 (d, J=3.5 Hz, 1H, Ar), 8.53-8.54 (d, J=7.5 Hz, 1H, CONH, exch), 10.13 (s, 1H, 3-NH, exch), 10.78 (s, 1H, 7-NH, exch).

(S)-2-({4-[4-(2-Amino-4-oxo-4,7-dihydro-3H-pyrrolo[2,3-d]pyrimidin-6-yl)-butyl]-thiophene-2-carbonyl}-amino)-pentanedioic acid diethyl ester (346): Compound 346 was prepared using the general method described for the preparation of 343-347, from 341 (200 mg, 0.39 mmol) to give 185 mg (92%) of 346 as a light yellow powder. mp 81-82° C.; TLC R_{f} 0.55 (CHCl₃/MeOH 5:1); ¹H NMR (DMSO-d₆): δ 1.17-1.19 (m, 6H, COOCH₂CH₃), 1.62 (m, 4H, CH₂CH₂), 1.95-2.12 (m, 2H, β-CH₂), 2.41-2.45 (t, J=7.5 Hz, 2H, γ-CH₂), 2.54-2.62 (m, 4H, CH₂CH₂), 4.03-4.07 (q, J=7.0 Hz, 2H, COOCH₂CH₃), 4.09-4.13 (m, 2H, COOCH₂CH₃), 4.36-4.40 (m, 1H, α-CH), 5.99 (s, 3H, C5-CH, 2-NH₂, exch), 7.41 (s, 1H, Ar), 7.76 (s, 1H, Ar), 8.67-8.68 (d, J=7.5 Hz, 1H, CONH, exch), 11.00 (s, 1H, 3-NH, exch), 11.39 (s, 1H, 7-NH, exch).

(S)-2-({5-[4-(2-Amino-4-oxo-4,7-dihydro-3H-pyrrolo[2,3-d]pyrimidin-6-yl)-butyl]-thiophene-3-carbonyl}-amino)-pentanedioic acid diethyl ester (347): Compound 347 was prepared using the general method described for the preparation of 343-347, from 342 (200 mg, 0.39 mmol) to give 183 mg (91%) of 347 as a light yellow powder. mp 82-83° C.; TLC R_{f} 0.54 (CHCl₃/MeOH 5:1); ¹H NMR (DMSO-d₆): δ 1.15-1.18 (m, 6H, COOCH₂CH₃), 1.64 (m, 4H, CH₂CH₂), 1.93-2.12 (m, 2H, β-CH₂), 2.41-2.44 (t, J=7.5 Hz, 2H, γ-CH₂), 2.55 (m, 2H, CH₂), 2.81 (m, 2H, CH₂), 4.03-4.07 (q, J=7.0 Hz, 2H, COOCH₂CH₃), 4.08-4.12 (q, J=7.0 Hz, 2H, COOCH₂CH₃), 4.36-4.40 (m, 1H, α-CH), 5.96 (s, 3H, C5-CH, 2-NH₂, exch), 7.26 (s, 1H, Ar), 7.98 (s, 1H, Ar), 8.46-8.48 (d, J=8.0 Hz, 1H, CONH, exch), 10.76 (s, 1H, 3-NH, exch), 11.22 (s, 1H, 7-NH, exch).

### General Procedure for the Synthesis of Compounds G114-G123.

To a solution of 338-347 (100 mg, 0.19 mmol) in MeOH (10 mL) was added 1 N NaOH (5 mL) and the mixture was stirred under N₂ at room temperature for 16 h. TLC showed the disappearance of the starting material (R_{f} 0.54, CHCl₃/MeOH 5:1) and one major spot at the origin. The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford G114-G123.

(S)-2-({2-[4-(2-Amino-4-oxo-4,7-dihydro-3H-pyrrolo[2,3-d]pyrimidin-6-yl)-butyl]-thiophene-3-carbonyl}-amino)-pentanedioic acid (G114): Compound G114 was prepared using the general method described for the preparation of G114-G123, from 343 (100 mg, 0.19 mmol) to give 83 mg (95%) of G114 as a light yellow powder. mp 181-182° C.; ¹H NMR (DMSO-d₆): δ 1.61 (m, 4H, CH₂CH₂), 1.87-2.10 (m, 2H, β-CH₂), 2.34-2.37 (t, J=7.5 Hz, 2H, γ-CH₂), 2.47 (m, 2H, CH₂), 3.09-3.11 (m, 2H, CH₂), 4.32-4.37 (m, 1H, α-CH), 5.86 (s, 1H, C5-CH), 5.98 (s, 2H, 2-NH₂, exch), 7.33-7.34 (d, J=5.0 Hz, 1H, Ar), 7.35-7.36 (d, J=5.0 Hz, 1H, Ar), 8.28-8.29 (d, J=7.5 Hz, 1H, CONH, exch), 10.16 (s, 1H, 3-NH, exch), 10.80 (s, 1H, 7-NH, exch), 12.36 (br, 2H, COOH, exch). Anal. calcd for (C₂₀H₂₃N₅O₆S.1.85H₂O): C, 48.55; H, 5.44; N, 14.15; S, 6.48; Found: C, 48.60; H, 5.09; N, 13.90; S, 6.23.

(S)-2-({3-[4-(2-Amino-4-oxo-4,7-dihydro-3H-pyrrolo[2,3-d]pyrimidin-6-yl)-butyl]-thiophene-2-carbonyl}-amino)-pentanedioic acid (G115): Compound G115 was prepared using the general method described for the preparation of G114-G123, from 344 (100 mg, 0.19 mmol) to give 83 mg (95%) of G115 as a light yellow powder. mp 180-181° C.; ¹H NMR (DMSO-d₆): δ 1.56 (m, 4H, CH₂CH₂), 1.88-2.11 (m, 2H, β-CH₂), 2.33-2.36 (t, J=7.5 Hz, 2H, γ-CH₂), 2.48 (m, 2H, CH₂), 2.86-2.88 (m, 2H, CH₂), 4.31-4.36 (m, 1H, α-CH), 5.85 (s, 1H, C5-CH), 5.98 (s, 2H, 2-NH₂, exch), 7.01-7.02 (d, J=5.0 Hz, 1H, Ar), 7.58-7.59 (d, J=5.0 Hz, 1H, Ar), 8.24-8.26 (d, J=7.5 Hz, 1H, CONH, exch), 10.16 (s, 1H, 3-NH, exch), 10.79 (s, 1H, 7-NH, exch), 12.38 (br, 2H, COOH, exch). Anal. calcd for (C₂₀H₂₃N₅O₆S.1.0H₂O): C, 50.10; H, 5.26; N, 14.61; S, 6.69; Found: C, 50.12; H, 5.18; N, 14.41; S, 6.43.

(S)-2-({4-[4-(2-Amino-4-oxo-4,7-dihydro-3H-pyrrolo[2,3-d]pyrimidin-6-371)-butyl]-thiophene-3-carbonyl}-amino)-pentanedioic acid (G116): Compound G116 was prepared using the general method described for the preparation of G114-G123, from 345 (100 mg, 0.19 mmol) to give 83 mg (95%) of G116 as a light yellow powder, mp 179-180° C.; NMR (DMSO-d₆): δ 1.56 (m, 4H, CH₂CH₂), 1.87-2.10 (m, 2H,)₃—CH₂), 2.35-2.38 (t, J=8.0 Hz, 2H, γ-CH₂), 2.47-2.48 (m, 2H, CH₂), 2.75-2.81 (m, 2H, CH₂), 4.32-4.36 (m, 1H, α-CH), 5.85 (s, 1H, C5-CH), 5.96 (s, 2H, 2-NH₂, exch), 7.19-7.20 (d, J=3.0 Hz, 1H, Ar), 7.92-7.93 (d, J=3.0 Hz, 1H, Ar), 8.40-8.41 (d, J=8.0 Hz, 1H, CONH, exch), 10.14 (s, 1H, 3-NH, exch), 10.78 (s, 1H, 7-NH, exch), 12.31 (br, 2H, COOH, exch). Anal, calcd for (C₂₀H₂₃N₅O₆S.0.4CHCl₃): C, 48.11; H, 4.63; N, 13.75; S, 6.30; Found: C, 48.15; H, 4.79; N, 13.66; S, 5.95.

(S)-2-({4-[4-(2-Amino-4-oxo-4,7-dihydro-3H-pyrrolo[2,3-d]pyrimidin-6-yl)-butyl]-thiophene-2-carbonyl}-amino)-pentanedioic acid (G117): Compound G117 was prepared using the general method described for the preparation of G114-G123, from 346 (100 mg, 0.19 mmol) to give 83 mg (95%) of G117 as a light yellow powder, mp 181-182° C.; ¹H NMR (DMSO-d₆): δ 1.61 (m, 4H, CH₂CH₂), 1.88-2.11 (m, 2H, β-CH₂), 2.33-2.36 (t, J=7.5 Hz, 2H, γ-CH₂), 2.56 (m, 4H, CH₂CH₂), 4.32-4.37 (m, 1H, α-CH), 5.88 (s, 1H, C5-CH), 5.96 (s, 2H, 2-NH₂, exch), 7.39 (s, 1H, Ar), 7.75 (s, 1H, Ar), 8.54-8.55 (d, J=8.0 Hz, 1H, CONH, exch), 10.13 (s, 1H, 3-NH, exch), 10.81 (s, 1H, 7-NH, exch), 12.45 (br, 2H, COOH, exch). Anal. calcd for (C₂₀H₂₃N₅O₆S.0.31CHCl₃): C, 48.93; H, 4.71; N, 14.04; S, 6.43; Found: C, 49.01; H, 5.04; N, 13.64; S, 6.31.

(S)-2-({5-[4-(2-Amino-4-oxo-4,7-dihydro-3H-pyrrolo[2,3-d]pyrimidin-6-yl)-butyl]-thiophene-3-carbonyl}-amino)-pentanedioic acid (G118): Compound G118 was prepared using the general method described for the preparation of G114-G123, from 347 (100 mg, 0.19 mmol) to give 83 mg (95%) of G118 as a light yellow powder. mp 177-178° C.; ¹H NMR (DMSO-d₆): δ 1.64 (m, 4H, CH₂CH₂), 1.87-2.10 (m, 2H, β-CH₂), 2.32-2.35 (t, J=7.5 Hz, 2H, γ-CH₂), 2.55 (m, 2H, CH₂), 2.80-2.82 (m, 2H, CH₂), 4.33-4.37 (m, 1H, α-CH), 5.88 (s, 1H, C5-CH), 5.96 (s, 2H, 2-NH₂, exch), 7.27 (s, 1H, Ar), 7.96 (s, 1H, Ar), 8.31-8.33 (d, J=8.0 Hz, 1H, CONH, exch), 10.12 (s, 1H, 3-NH, exch), 10.80 (s, 1H, 7-NH, exch), 12.43 (br, 2H, COOH, exch). Anal. calcd for (C₂₀H₂₃N₅O₆S.1.5H₂O): C, 49.17; H, 5.36; N, 14.34; S, 6.56; Found: C, 48.85; H, 4.96; N, 14.00; S, 6.49.

(S)-2-({2-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid (G119): Compound G119 was prepared using the general method described for the preparation of G114-G123, from 338 (100 mg, 0.19 mmol) to give 84 mg (95%) of G119 as a light yellow powder. mp 175-176° C.; ¹H NMR (DMSO-d₆): δ 1.91-2.13 (m, 2H, β-CH₂), 2.34-2.37 (t, J=7 Hz, 2H, γ-CH₂), 2.81 (m, 4H, CH₂CH₂), 4.41-4.45 (m, 1H, α-CH), 6.01 (s, 3H, C5-CH, 2-NH, exch), 7.37-7.38 (d, J=5.5 Hz, 1H, Ar), 7.53-7.54 (d, J=5.5 Hz, 1H, Ar), 8.25-8.26 (d, J=7.5 Hz, 1H, CONH, exch), 10.19 (s, 1H, 3-NH, exch), 10.88 (s, 1H, 7-NH, exch), 12.47 (br, 2H, COOH, exch). Anal. calcd for (C₂₀H₁₉N₅O₆S.1.0CH₃COOH): C, 51.06; H, 4.48; N, 13.53; S, 6.20; Found: C, 50.66; H, 4,22; N, 13.57; S, 6.25.

(S)-2-({3-[4-(2-Amino-4-oxo-4,7-dihydro-3H-pyrrolo[2,3-d]pyrimidin-6-yl)-but-1-ynyl]-thiophene-2-carbonyl}-amino)-pentanedioic acid (G120): Compound G120 was prepared using the general method described for the preparation of G114-G123, from 339 (100 mg, 0.19 mmol) to give 83 mg (94%) of G120 as a light yellow powder. mp 177-178° C.; ¹H NMR (DMSO-d₆): δ 1.96-2.18 (m, 2H, β-CH₂), 2.31-2.34 (t, J=7.5 Hz, 2H, γ-CH₂), 2.84 (m, 4H, CH₂CH₂), 4.41-4.53 (m, 1H, α-CH), 6.01 (s, 3H, C5-CH, 2-NH₂, exch), 7.16-7.17 (d, J=5.0 Hz, 1H, Ar), 7.80-7.81 (d, J=5.0 Hz, 1H, Ar), 8.22-8.23 (d, J=7.5 Hz, 1H, CONH, exch), 10.20 (s, 1H, 3-NH, exch), 10.89 (s, 1H, 7-NH, exch), 12.30 (br, 2H, COOH, exch). Anal. calcd for (C₂₀H₁₉N₅O₆S.0.3CHCl₃): C, 49.43; H, 3.94; N, 14.20; S, 6.50; Found: C, 49.71; H, 4.30; N, 14.01; S, 6.31.

(S)-2-({4-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid (G121): Compound G121 was prepared using the general method described for the preparation of G114-G123, from 340 (100 mg, 0.19 mmol) to give 84 mg (95%) of G121 as a light yellow powder. mp 181-182° C.; ¹H NMR (DMSO-d₆): δ 1.92-2.14 (m, 2H, β-CH₂), 2.33-2.37 (t, J=8.0 Hz, 2H, γ-CH₂), 2.70-2.80 (m, 4H, CH₂CH₂), 4.45-4.50 (m, 1H, α-CH), 6.00 (s, 3H, C5-CH, 2-NH₂, exch), 7.74-7.75 (d, J=3.0 Hz, 1H, Ar), 8.10-8.11 (d, J=3.0 Hz, 1H, Ar), 8.30-8.32 (d, J=8.0 Hz, 1H, CONH, exch), 10.17 (s, 1H, 3-NH, exch), 10.86 (s, 1H, 7-NH, exch), 12.44 (br, 2H, COOH, exch). Anal, calcd for (C₂₀H₁₉N₅O₆S.0.3CHCl₃): C, 49.43; H, 3.94; N, 14.20; S, 6.50; Found: C, 49.80; H, 4.14; N, 13.95; S, 6.15.

(S)-2-({4-[4-(2-Amino-4-oxo-4,7-dihydro-3H-pyrrolo[2,3-d]pyrimidin-6-yl)-but-1-ynyl]-thiophene-2-carbonyl}-amino)-pentanedioic acid (G122): Compound G122 was prepared using the general method described for the preparation of G114-G123, from 341 (100 mg, 0.19 mmol) to give 85 mg (96%) of G122 as a light yellow powder. mp 178-179° C.; ¹H NMR (DMSO-d₆): δ 1.91-2.11 (m, 2H, β-CH₂), 2.34-2.37 (t, J=7.5 Hz, 2H, γ-CH₂), 2.71-2.79 (m, 4H, CH₂CH₂), 4.32-4.36 (m, 1H, α-CH), 6.01 (s, 3H, C5-CH, 2-NH₂, exch), 7.86-7.88 (m, 2H, Ar), 8.64-8.65 (d, J=7.5 Hz, 1H, CONH, exch), 10.16 (s, 1H, 3-NH, exch), 10.88 (s, 1H, 7-NH, exch), 12.46 (br, 2H, COOH, exch). Anal. calcd for (C₂₀H₁₉N₅O₆S.0.37CH₂Cl₂): C, 50.04; H, 4.07; N, 14.33; S, 6.56; Found: C, 50.40; H, 4.13; N, 13.95; S, 6.53.

(S)-2-({5-[4-(2-Amino-4-oxo-4,7-dihydro-3H-pyrrolo[2,3-d]pyrimidin-6-yl)-but-1-ynyl]-thiophene-3-carbonyl}-amino)-pentanedioic acid (G123): Compound G123 was prepared using the general method described for the preparation of G114-G123, from 342 (100 mg, 0.19 mmol) to give 84 mg (95%) of G123 as a light yellow powder, mp 176-177° C.; ¹H NMR (DMSO-d₆): δ 1.92-2.14 (m, 2H, β-CH₂), 2.33-2.37 (t, J=8.0 Hz, 2H, γ-CH₂), 2.70-2.80 (m, 4H, CH₂CH₂), 4.45-4.50 (m, 1H, α-CH), 6.00 (s, 3H, C5-CH, 2-NH₂, exch), 7.74-7.75 (d, J=3.0 Hz, 1H, Ar), 8.10-8.11 (d, J=3.0 Hz, 1H, Ar), 8.30-8.32 (d, J=8.0 Hz, 1H, CONH, exch), 10.17 (s, 1H, 3-NH, exch), 10.86 (s, 1H, 7-NH, exch), 12.44 (br, 2H, COOH, exch). Anal. calcd for (C₂₀H₁₉N₅O₆S.0.41CH₂Cl₂): C, 49.80; H, 4.06; N, 14.23; S, 6.51; Found: C, 50.02; H, 4.21; N, 13,83; S, 6.41.

4-(4-Hydroxy-but-1-ynyl)-thiophene-2-carboxylic acid methyl ester (408): To a solution 4-bromo-thiophene-2-carboxylic acid methyl ester (4.42 g, 20 mmol) in anhydrous acetonitrile (20 mL), was added palladium chloride (142 mg, 0.8 mmol), triphenylphosphine (261 mg, 0.8 mmol), copper iodide (608 mg, 3.2 mmol), triethylamine (20.2 g, 0.2 mol) and but-3-yn-1-ol, 280 (2.1 g, 30 mmol). The reaction mixture was heated to 100° C. for 6 h. Then, silica gel (10 g) was added, and the solvent was evaporated to afford a plug under reduced pressure. The resulting plug was loaded on to a silica gel column (3.5×12 cm) and eluted with hexane followed by 50% EtOAc in hexane. The desired fractions (TLC) were pooled and evaporated to afford 2.8 g (67%) of 408 as a light yellow oil. TLC R_{f} 0.33 (hexane/EtOAc 1:1); ¹H NMR (DMSO-d₆); δ 2.52-2.55 (t, J=6.8 Hz, 2H, CH₂), 3.56-3.59 (t, J=6.8 Hz, 2H, CH₂), 3.83 (s, 3H, COOCH₃), 7.73 (s, 1H, Ar), 8.03 (s, 1H, Ar).

5-(4-Hydroxy-but-1-ynyl)-thiophene-3-carboxylic acid methyl ester (409): To a solution 5-bromo-thiophene-3-carboxylic acid methyl ester (4.42 g, 20 mmol) in anhydrous acetonitrile (20 mL), was added palladium chloride (142 mg, 0.8 mmol), triphenylphosphine (261 mg, 0.8 mmol), copper iodide (608 mg, 3.2 mmol), triethylamine (20.2 g, 0.2 mol) and but-3-yn-1-ol, 280 (2.1 g, 30 mmol). The reaction mixture was heated to 100° C. for 6 h. Then, silica gel (10 g) was added, and the solvent was evaporated to afford a plug under reduced pressure. The resulting plug was loaded on to a silica gel column (3.5×12 cm) and eluted with hexane followed by 50% EtOAc in hexane. The desired fractions (TLC) were pooled and evaporated to afford 2.96 g (71%) of 409 as a light yellow oil. TLC R_{f} 0.33 (hexane/EtOAc 1:1); NMR (DMSO-d₆): δ 2.58-2.61 (t, J=6.5 Hz, 2H, CH₂), 3.56-3.59 (t, J=6.5 Hz, 2H, CH₂), 3.80 (s, 3H, COOCH₃), 4.93-4.95 (t, J=5.5 Hz, 1H, OH, exch), 7.47 (s, J=1.5 Hz, 1H, Ar), 8.28 (d, J=1.5 Hz, 1H, Ar).

4-(4-Hydroxy-butyl)-thiophene-2-carboxylic acid methyl ester (410): To a Parr flask was added 408 (2.8 g, 13 mmol), 10% palladium on activated carbon (1.4 g), and MeOH (50 mL). Hydrogenation was carried out at 55 psi of H₂ for 4 h. The reaction mixture was filtered through Celite, washed with MeOH/CHCl₃ (1:1) (100 mL), passed through a short silica gel column (3×5 cm), and concentrated under reduced pressure to give 2.78 g (99%) of 410 as a yellow oil. TLC R_{f} 0.34 (hexane/EtOAc 1:1); NMR (DMSO-d₆): δ 1.38-1.45 (m, 2H, CH₂), 1.56-1.64 (m, 2H, CH₂), 2.58-2.62 (t, J=7.2 Hz, 2H, CH₂), 3.39-3.42 (t, J=6.8 Hz, 2H, CH₂), 3.81 (s, 3H, COOCH₃), 7.57 (s, 1H, Ar), 7.67 (s, 1H, Ar).

5-(4-Hydroxy-butyl)-thiophene-3-carboxylic acid methyl ester (411): To a Parr flask was added 409 (2.96 g, 13.7 mmol), 10% palladium on activated carbon (1.5 g), and MeOH (50 mL). Hydrogenation was carried out at 55 psi of H₂ for 4 h. The reaction mixture was filtered through Celite, washed with MeOH/CHCl₃ (1:1) (100 mL), passed through a short silica gel column (3×5 cm), and concentrated under reduced pressure to give 2.78 g (91%) of 411 as a yellow oil, TLC R_{f} 0.34 (hexane/EtOAc 1:1); ¹H NMR (DMSO-d₆): δ 1.43-1.49 (m, 2H, CH₂), 1.60-1.68 (m, 2H, CH₂), 2.79-2.82 (t, J=7.2 Hz, 2H, CH₂), 3.40-3.42 (t, J=6.5 Hz, 2H, CH₂), 3.78 (s, 3H, COOCH₃), 4.41 (s, 1H, OH, exch), 7.18 (s, 1H, Ar), 8.13 (s, 1H, Ar).

4-(3-Carboxy-propyl)-thiophene-2-carboxylic acid methyl ester (412): To acetonitrile (40 mL) was added periodic acid (6.52 g, 28.6 mmol) and the mixture was stirred vigorously for 15 min. Compound 410 (2.78 g, 13 mmol) was the added (in ice-water bath) followed by addition of PCC (56 mg, 0.26 mmol) and the reaction mixture was stirred for 1 h. The solvent was evaporated under reduce pressure to afford a residue. The resulting residue was then diluted with EtOAc (100 mL) and washed with bine-water, sat. aq NaHSO₃ solution, and brine, respectively, dried over anhydrous Na₂SO₄ and concentrated to give 2.34 g (79%) of 412 as a light yellow oil. TLC R_{f} 0.58 (hexane/EtOAc 1:1); ¹HNMR (DMSO-d₆): δ 1.78-1.84 (m, 2H, CH₂), 2.20-2.23 (t, J=7.5 Hz, 2H, CH₂), 2.60-2.63 (t, J=7.5 Hz, 2H, CH₂), 3.81 (s, 3H, COOCH₃), 7.59 (s, 1H, Ar), 7.68 (s, 1H, Ar), 12.07 (br, 1H, COOH, exch).

5-(3-Carboxy-propyl)-thiophene-3-carboxylic acid methyl ester (413): To acetonitrile (40 mL) was added periodic acid (6.52 g, 28.6 mmol) and the mixture was stirred vigorously for 15 min. Compound 411 (2.78 g, 13 mmol) was the added (in ice-water bath) followed by addition of PCC (56 mg, 0.26 mmol) and the reaction mixture was stirred for 1 h. The solvent was evaporated under reduce pressure to afford a residue. The resulting residue was then diluted with EtOAc (100 mL) and washed with bine-water, sat, aq NaHSO₃ solution, and brine, respectively, dried over anhydrous Na₂SO₄ and concentrated to give 3.01 g (99%) of 413 as a light yellow oil. TLC R_{f} 0.58 (hexane/EtOAc 1:1); ¹H NMR (DMSO-d₆): δ 1.80-1.87 (m, 2H, CH₂), 2.25-2.28 (t, J=7.2 Hz, 2H, CH₂), 2.81-2.84 (t, J=7.2 Hz, 214, CH₂), 3.78 (s, 3H, COOCH₃), 7.20 (s, 1H, Ar), 8.15 (s, 1H, Ar), 12.15 (br, 1H, COOH, exch).

4-[3-(2-Amino-4-oxo-4,7-dihydro-3H-pyrrolo[2,3-d]pyrimidin-6-yl)-propyl]-thiophene-2-carboxylic acid methyl ester (420): To 412 (2.28 g, 10 mmol) in a 100 mL flask were added oxalyl chloride (7.6 g, 60 mmol) and anhydrous CH₂Cl₂ (20 mL). The resulting solution was refluxed for 1 h and then cooled to room temperature. After evaporating the solvent under reduced pressure, the residue was dissolved in 20 mL of Et₂O. The resulting solution was added dropwise to an ice-cooled diazomethane (generated in situ from 10 g of diazald by using Aldrich Mini Diazald Apparatus) in an ice bath over 10 min. The resulting mixture was allowed to stand for 30 min and then stirred for an additional 1 h. To this solution was added conc. HCl (20 mL). The resulting mixture was refluxed for 1.5 h. After cooling to room temperature, the organic layer was separated and the aqueous layer extracted with Et₂O (50 mL×2). The combined organic layer and Et₂O extract was washed with two portions of 10% Na₂CO₃ solution and dried over Na₂SO₄. Evaporation of the solvent afforded a light yellow residue. To this residue in anhydrous DMF (15 mL) was added 2,6-diamino-3H-pyrimidin-4-one, 24 (1.26 g, 10 mmol). The resulting mixture was stirred under N₂ at 50-60° C. for 3 days. Silica gel (2 g) was then added, and the solvent was evaporated under reduced pressure. The resulting plug was loaded on to a silica gel column (2.5×12 cm) and eluted with CHCl₃ followed by 2% MeOH in CHCl₃ and then 4% MeOH in CHCl₃. Fractions with an R_{f} 0.17 (CHCl₃/MeOH 10:1) were pooled and evaporated to afford 920 mg (28%) of 420 as a light yellow powder, mp 182-183° C.; ¹H NMR (DMSO-d₆): δ 1.87-1.94 (m, 2H, CH₂), 2.49-2.52 (t, J=7.6 Hz, 2H, CH₂), 2.60-2.64 (t, J=7.6 Hz, 2H, CH₂), 3.81 (s, 3H, COOCH₃), 5.95 (d, J=2.0 Hz, 1H, C5-CH), 6.43 (s, 2H, 2-NH₂, exch), 7.60 (d, J=1.6 Hz, 1H, Ar), 7.70 (d, J=1.6 Hz, 1H, Ar), 10.55 (s, 1H, 3-NH, exch), 11.08 (s, 1H, 7-NH, exch).

4-[3-(2,4-Diamino-furo[2,3-d]pyrimidin-5-yl)-propyl]-thiophene-2-carboxylic acid methyl ester (422): Fractions with an R_{f} 0.25 (CHCl₃/MeOH 10:1) were pooled and evaporated to afford 320 mg (9.6%) of 422 as a light yellow powder. mp 156-157° C.; ¹H NMR (DMSO-d₆): δ 1.81-1.88 (m, 2H, CH₂), 2.63-2.69 (m, 4H, CH₂, CH₂), 3.81 (s, 3H, COOCH₃), 5.97 (s, 2H, 2-NH₂, exch), 6.41 (s, 2H, 4-NH₂, exch), 7.14 (s, 1H, C6-CH), 7.61 (d, J=1.2 Hz, 1H, Ar), 7.71 (d, J=1.2 Hz, 1H, Ar).

5-[3-(2-Amino-4-oxo-4,7-dihydro-3H-pyrrolo[2,3-d]pyrimidin-6-yl)-propyl]-thiophene-3-carboxylic acid methyl ester (421): To 413 (2.28 g, 10 mmol) in a 100 mL flask were added oxalyl chloride (7.6 g, 60 mmol) and anhydrous CH₂Cl₂ (20 mL). The resulting solution was refluxed for 1 h and then cooled to room temperature. After evaporating the solvent under reduced pressure, the residue was dissolved in 20 mL of Et₂O. The resulting solution was added dropwise to an ice-cooled diazomethane (generated in situ from 10 g of diazald by using Aldrich Mini Diazald Apparatus) in an ice bath over 10 min. The resulting mixture was allowed to stand for 30 min and then stirred for an additional 1 h. To this solution was added conc. HCl (20 mL). The resulting mixture was refluxed for 1.5 h. After cooling to room temperature, the organic layer was separated and the aqueous layer extracted with Et₂O (50 mL×2). The combined organic layer and Et₂O extract was washed with two portions of 10% Na₂CO₃ solution and dried over Na₂SO₄. Evaporation of the solvent afforded a light yellow residue. To this residue in anhydrous DMF (15 mL) was added 2,6-diamino-3H-pyrimidin-4-one, 24 (1.26 g, 10 mmol). The resulting mixture was stirred under N₂ at 50-60° C. for 3 days. Silica gel (2 g) was then added, and the solvent was evaporated under reduced pressure. The resulting plug was loaded on to a silica gel column (2.5×12 cm) and eluted with CHCl₃ followed by 2% MeOH in CHCl₃ and then 4% MeOH in CHCl₃. Fractions with an R_{f} 0.17 (CHCl₃/MeOH 10:1) were pooled and evaporated to afford 986 mg (30%) of 421 as a light yellow powder. mp 182-183° C.; ¹H NMR (DMSO-d₆): δ 1.90-1.97 (m, 2H, CH₂), 2.51-2.56 (t, J=7.2 Hz, 2H, CH₂), 2.80-2.83 (t, J=7.2 Hz, 2H, CH₂), 3.78 (s, 3H, COOCH₃), 5.90 (d, J=1.6 Hz, 1H, C5-CH), 5.98 (s, 2H, 2-NH₂, exch), 7.21 (s, 1H, Ar), 8.14 (s, 1H, Ar), 10.15 (s, 1H, 3-NH, exch), 10.84 (s, 1H, 7-NH, exch),

5-[3-(2,4-Diamino-furo[2,3-d]pyrimidin-5-yl)-propyl]-thiophene-3-carboxylic acid methyl ester (423): Fractions with an R_{f} 0.25 (CHCl₃/MeOH 10:1) were pooled and evaporated to afford 327 mg (9.8%) of 423 as a light yellow powder. mp 156-157° C.; ¹H NMR (DMSO-d₆): δ 1.83-1.92 (m, 2H, CH₂), 2.66-2.70 (t, J=7.6 Hz, 2H, CH₂), 2.86-2.89 (t, J=7.6 Hz, 2H, CH₂), 3.78 (s, 3H, COOCH₃), 5.99 (s, 2H, 2-NH₂, exch), 6.44 (s, 2H, 4-NH₂, exch), 7.15 (s, 1H, C6-CH), 7.22 (d, J=1.2 Hz, 1H, Ar), 8.14 (d, J=1.2 Hz, 1H, Ar).

4-[3-(2-Amino-4-oxo-4,7-dihydro-3H-pyrrolo[2,3-d]pyrimidin-6-yl)-propyl]-thiophene-2-carboxylic acid (424): To a solution of 420 (460 mg, 1.4 mmol) in MeOH (10 mL) was added 1 N NaOH (10 mL) and the mixture was stirred under N₂ at 40° C. for 16 h. TLC showed the disappearance of the starting material (R_{f} 0.16) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 367 mg (83%) of 424 as a yellow powder. mp 181-182° C.; ¹H NMR (DMSO-d₆): δ 1.86-1.93 (m, 2H, CH₂), 2.48-2.52 (t, J=7.6 Hz, 2H, CH₂), 2.59-162 (t, J=7.6 Hz, 2H, CH₂), 5.90 (s, 1H, C5-CH), 6.01 (s, 2H, 2-NH₂, exch), 7.53 (s, 1H, Ar), 7.62 (s, 1H, Ar), 10.17 (s, 1H, 3-NH, exch), 10.84 (s, 1H, 7-NH, exch), 12.98 (br, 1H, COOH, exch).

5-[3-(2-Amino-4-oxo-4,7-dihydro-3H-pyrrolo[2,3-d]pyrimidin-6-yl)-propyl]-thiophene-3-carboxylic acid (425): To a solution of 421 (460 mg, 1.4 mmol) in MeOH (10 mL) was added 1 N NaOH (10 mL) and the mixture was stirred under N₂ at 40° C. for 16 h. TLC showed the disappearance of the starting material (R_{f} 0.16) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 376 mg (85%) of 425 as a yellow powder. mp 181-182° C.; ¹H NMR (DMSO-d₆): δ 1.89-1.97 (m, 2H, CH₂), 2.51-2.56 (t, J=7.6 Hz, 2H, CH₂), 2.78-2.82 (t, J=7.6 Hz, 2H, CH₂), 5.90 (s, 1H, C5-CH), 6.00 (s, 2H, 2-NH₂, exch), 7.17 (s, J=1.2 Hz, 1H, Ar), 8.05 (s, J=1.2 Hz, 1H, Ar), 10.17 (s, 1H, 3-NH, exch), 10.85 (s, 1H, 7-NH, exch), 12.62 (br, 1H, COOH, exch).

4-[3-(2,4-Diamino-furo[2,3-d]pyrimidin-5-yl)-propyl]-thiophene-2-carboxylic acid (426): To a solution of 422 (320 mg, 0.96 mmol) in MeOH (10 mL) was added 1 N NaOH (10 mL) and the mixture was stirred under N₂ at 40° C. for 16 h. TLC showed the disappearance of the starting material (R_{f} 0.25) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 280 mg (92%) of 426 as a light yellow powder. mp 156-157° C.; ¹H NMR (DMSO-d₆): δ 1.80-1.88 (m, 2H, CH₂), 2.63-2.68 (m, 4H, CH₂, CH₂), 5.97 (s, 2H, 2-NH₂, exch), 6.41 (s, 2H, 4-NH₂, exch), 7.14 (s, 1H, C6-CH), 7.52 (d, J=1.6 Hz, 1H, Ar), 7.61 (d, J=1.6 Hz, 1H, Ar), 12.99 (br, 1H, COOH, exch), 4-[3-(2,4-Diamino-furo[2,3-d]pyrimidin-5-yl)-propyl]-thiophene-2-carboxylic acid (427): To a solution of 423 (320 mg, 0.96 mmol) in MeOH (10 mL) was added 1 N NaOH (10 mL) and the mixture was stirred under N₂ at 40° C. for 16 h. TLC showed the disappearance of the starting material (R_{f} 25) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 283 mg (93%) of 427 as a light yellow powder. mp 156-157° C.; ¹H NMR (DMSO-d₆): δ 1.81-1.91 (m, 2H, CH₂), 2.66-2.70 (t, J=7.6 Hz, 2H, CH₂), 2.85-2.88 (t, J=7.6 Hz, 2H, CH₂), 5.98 (s, 2H, 2-NH₂, exch), 6.44 (s, 2H, 4-NH₂, exch), 7.15 (s, 1H, C6-CH), 7.17 (d, J=1.6 Hz, 1H, Ar), 8.03 (d, J=1.2 Hz, 1H, Ar), 12.63 (br, 1H, COOH, exch).

(S)-2-({4-[3-(2-Amino-4-oxo-4,7-dihydro-3H-pyrrolo[2,3-d]pyrimidin-6-yl)-propyl]-thiophene-2-carbonyl}-amino)-pentanedioic acid diethyl ester (428): To a solution of 424 (159 mg, 0.5 mmol) in anhydrous DMF (10 mL) were added N-methylmorpholine (91 mg, 0.9 mmol) and 2-chloro-4,6-dimethoxy-1,3,5-triazine (158 mg, 0.9 mmol). The resulting mixture was stirred at room temperature for 2 h. To this mixture were added N-methylmorpholine (91 mg, 0.9 mmol) and L-glutamate diethyl ester hydrochloride (180 mg, 0.75 mmol). The reaction mixture was stirred for an additional 4 h at room temperature and then evaporated to dryness under reduced pressure. The residue was dissolved in the minimum amount of CHCl₃/MeOH (4:1) and chromatographed on a silica gel column (1.5×15 cm) and with 5% CHCl₃ in MeOH as the eluent. Fractions that showed the desired spot (TLC) were pooled and the solvent evaporated to dryness to afford 220 mg (87%) of 428 as a yellow powder, mp 81-82° C.; TLC R_{f} 0.13 (CHCl₃/MeOH 10:1); ¹H NMR (DMSO-d₆): δ 1.15-1.21 (m, 6H, COOCH₂CH₃), 1.87-2.14 (m, 4H, β-CH₂, CH₂), 2.41-2.45 (t, J=7.6 Hz, 2H, γ-CH₂), 2.51-2.55 (t, J=7.6 Hz, 2H, CH₂), 2.59-2.62 (t, J=7.6 Hz, 2H, CH₂), 4.02-4.14 (m, 4H, COOCH₂CH₃), 4.36-4.41 (m, 1H, α-CH), 5.92-5.93 (d, J=2.0 Hz, 1H, C5-CH), 6.14 (s, 2H, 2-NH₂, exch), 7.44 (d, J=1.2 Hz, 1H, Ar), 7.78 (d, J=1.2 Hz, 1H, Ar), 8.68-8.70 (d, J=7.2 Hz, 1H, CONH, exch), 10.28 (s, 1H, 3-NH, exch), 10.90 (s, 1H, 7-NH, exch).

(S)-2-({5-[3-(2-Amino-4-oxo-4,7-dihydro-3H-pyrrolo[2,3-d]pyrimidin-6-yl)-propyl]-thiophene-3-carbonyl}-amino)-pentanedioic acid diethyl ester (429): To a solution of 425 (159 mg, 0.5 mmol) in anhydrous DMF (10 mL) were added N-methylmorpholine (91 mg, 0.9 mmol) and 2-chloro-4,6-dimethoxy-1,3,5-triazine (158 mg, 0.9 mmol). The resulting mixture was stirred at room temperature for 2 h. To this mixture were added N-methylmorpholine (91 mg, 0.9 mmol) and L-glutamate diethyl ester hydrochloride (180 mg, 0.75 mmol). The reaction mixture was stirred for an additional 4 h at room temperature and then evaporated to dryness under reduced pressure. The residue was dissolved in the minimum amount of CHCl₃/MeOH (4:1) and chromatographed on a silica gel column (1.5×15 cm) and with 5% CHCl₃ in MeOH as the eluent. Fractions that showed the desired spot (TLC) were pooled and the solvent evaporated to dryness to afford 223 mg (88%) of 429 as a yellow powder. mp 81-82° C.; TLC R_{f} 0.13 (CHCl₃/MeOH 10:1); ¹H NMR (DMSO-d₆): δ 1.15-1.21 (m, 6H, COOCH₂CH₃), 1.87-2.11 (m, 4H, β-CH₂, CH₂), 2.41-2.44 (t, J=7.6 Hz, 2H, γ-CH₂), 2.54-2.58 (t, J=7.6 Hz, 2H, CH₂), 2.78-2.82 (t, J=7.6 Hz, 2H, CH₂), 4.02-4.13 (m, 4H, COOCH₂CH₃), 4.36-4.41 (m, 1H, α-CH), 5.94 (d, J=2.0 Hz, 1H, C5-CH), 6.34 (s, 2H, 2-NH₂, exch), 7.29 (d, J=1.2 Hz, 1H, Ar), 8.01 (d, J=1.2 Hz, 1H, Ar), 8.49-8.51 (d, J=7.6 Hz, 1H, CONH, exch), 10.46 (s, 1H, 3-NH, exch), 1.18 (s, 1H, 7-NH, exch).

(S)-2-({4-[3-(2,4-Diamino-furo[2,3-d]pyrimidin-5-yl)-propyl]-thiophene-2-carbonyl}-amino)-pentanedioic acid diethyl ester (430): To a solution of 426 (159 mg, 0.5 mmol) in anhydrous DMF (10 mL) were added N-methylmorpholine (91 mg, 0.9 mmol) and 2-chloro-4,6-dimethoxy-1,3,5-triazine (158 mg, 0.9 mmol). The resulting mixture was stirred at room temperature for 2 h. To this mixture were added N-methylmorpholine (91 mg, 0.9 mmol) and L-glutamate diethyl ester hydrochloride (180 mg, 0.75 mmol). The reaction mixture was stirred for an additional 4 h at room temperature and then evaporated to dryness under reduced pressure. The residue was dissolved in the minimum amount of CHCl₃/MeOH (4:1) and chromatographed on a silica gel column (1.5×15 cm) and with 2% CHCl₃ in MeOH as the eluent. Fractions that showed the desired spot (TLC) were pooled and the solvent evaporated to dryness to afford 200 mg (80%) of 430 as a yellow powder. mp 79-80° C.; TLC R_{f} 0.26 (CHCl₃/MeOH 10:1); ¹H NMR (DMSO-d₆): δ 1.15-1.21 (m, 6H, COOCH₂CH₃), 1.82-2.12 (m, 4H, 18-CH₂, CH₂), 2.41-2.45 (t, J=7.2 Hz, 2H, γ-CH₂), 2.64-2.70 (m, 4H, CH₂, CH₂), 4.02-4.14 (m, 4H, COOCH₂CH₃), 4.36-4.42 (m, 1H, α-CH), 5.97 (s, 2H, 2-NH₂, exch), 6.41 (s, 2H, 4-NH₂, exch), 7.16 (s, 1H, C6-CH), 7.44 (d, J=1.2 Hz, 1H, Ar), 7.76 (d, J=1.2 Hz, 1H, Ar), 8.65-8.67 (d, J=7.6 Hz, 1H, CONH, exch).

(S)-2-({5-[3-(2,4-Diamino-furo[2,3-d]pyrimidin-5-yl)-propyl]-thiophene-3-carbonyl}-amino)-pentanedioic acid diethyl ester (431): To a solution of 427 (159 mg, 0.5 mmol) in anhydrous DMF (10 mL) were added N-methylmorpholine (91 mg, 0.9 mmol) and 2-chloro-4,6-dimethoxy-1,3,5-triazine (158 mg, 0.9 mmol). The resulting mixture was stirred at room temperature for 2 h. To this mixture were added N-methylmorpholine (91 mg, 0.9 mmol) and glutamate diethyl ester hydrochloride (180 mg, 0.75 mmol). The reaction mixture was stirred for an additional 4 h at room temperature and then evaporated to dryness under reduced pressure. The residue was dissolved in the minimum amount of CHCl₃/MeOH (4:1) and chromatographed on a silica gel column (1.5×15 cm) and with 2% CHCl₃ in MeOH as the eluent. Fractions that showed the desired spot (TLC) were pooled and the solvent evaporated to dryness to afford 205 mg (80%) of 431 as a yellow powder. mp 79-80° C.; TLC R_{f} 026 (CHCl₃/MeOH 10:1); ¹H NMR (DMSO-d₆): δ 1.15-1.21 (m, 6H, COOCH₂CH₃), 1.82-2.13 (m, 4H, β-CH₂, CH₂), 2.40-2.44 (t, J=7.6 Hz, 2H, γ-CH₂), 2.68-2.71 (t, J=7.2 Hz, 2H, CH₂), 2.84-2.88 (t, J=7.2 Hz, 2H, CH₂), 4.02-4.13 (m, 4H, COOCH₂CH₃), 4.36-4.41 (m, 1H, α-CH), 5.99 (s, 2H, 2-NH₂, exch), 6.45 (s, 2H, 4-NH₂, exch), 7.16 (s, 1H, C6-CH), 7.29 (d, J=1.2 Hz, 1H, Ar), 7.98 (d, J=1.2 Hz, 1H, Ar), 8.65-8.47 (d, J=7.6 Hz, 1H, CONH, exch).

(S)-2-({4-[3-(2-Amino-4-oxo-4,7-dihydro-3H-pyrrolo[2,3-d]pyrimidin-6-yl)-propyl]-thiophene-2-carbonyl}-amino)-pentanedioic acid (G150): To a solution of 428 (220 mg, 0.45 mmol) in MeOH (10 mL) was added 1 N NaOH (10 mL) and the mixture was stirred under N₂ at room temperature for 16 h. TLC showed the disappearance of the starting material (R_{f} 0.13) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 125 mg (62%) of G150 as yellow powder, mp 191-192° C.; ¹H NMR (DMSO-d₆): δ 1.87-2.12 (m, 4H, β-CH₂, CH₂), 2.33-2.37 (t, J=7.2 Hz, 2H, γ-CH₂), 2.51-2.53 (t, J=7.2 Hz, 2H, CH₂), 2.59-2.62 (t, J=7.2 Hz, 2H, CH₂), 4.32-4.37 (m, 1H, α-CH), 5.92 (d, J=2.0 Hz, 1H, C5-CH), 5.98 (s, 2H, 2-NH₂, exch), 7.43 (d, J=1.2 Hz, 1H, Ar), 7.77 (d, J=1.2 Hz, 1H, Ar), 8.55-8.57 (d, J=7.6 Hz, 1H, CONH, exch), 10.14 (s, 1H, 3-NH, exch), 10.84 (s, 1H, 7-NH, exch) 12.45 (br, 2H, COOH, exch). Anal. calcd for (C₁₉H₂₁N₅O₆S.1.7H₂O): C, 47.73; H, 5.14; N, 14.65; S, 6.71; Found: C, 47.75; H, 4.95; N, 14.38; S, 6.59.

(S)-2-({5-[3-(2-Amino-4-oxo-4,7-dihydro-3H-pyrrolo[2,3-d]pyrimidin-6-yl)-propyl]-thiophene-3-carbonyl}-amino)-pentanedioic acid (G154): To a solution of 429 (220 mg, 0.45 mmol) in MeOH (10 mL) was added 1 N NaOH (10 mL) and the mixture was stirred under N₂ at room temperature for 16 h. TLC showed the disappearance of the starting material (R_{f} 0.13) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 131 mg (65%) of G154 as yellow powder. mp 191-192° C.; ¹H NMR (DMSO-d₆): δ 1.87-2.10 (m, 4H, β-CH₂, CH₂), 2.33-2.36 (t, J=7.2 Hz, 2H, γ-CH₂), 2.55-2.57 (t, J=7.2 Hz, 2H, CH₂), 2.79-2.82 (t, J=7.2 Hz, 2H, CH₂), 4.35 (m, 1H, α-CH), 5.92 (s, 1H, C5-CH), 6.03 (s, 2H, 2-NH₂, exch), 7.30 (s, 1H, Ar), 7.99 (s, 1H, Ar), 8.35-8.37 (d, J=7.2 Hz, 1H, CONH, exch), 10.19 (s, 1H, 3-NH, exch), 10.87 (s, 1H, 7-NH, exch) 12.41 (br, 2H, COOH, exch), Anal. calcd for (C₁₉H₂₁N₅O₆S.1.4H₂O): C, 48.28; H, 5.08; N, 14.82; S, 6.78; Found: C, 48.23; H, 4.90; N, 14.74; S, 6.73.

(S)-2-({4-[3-(2,4-Diamino-furo[2,3-d]pyrimidin-5-yl)-propyl]-thiophene-2-carbonyl}-amino)-pentanedioic acid (G152): To a solution of 430 (200 mg, 0.41 mmol) in MeOH (10 mL) was added 1 N NaOH (10 mL) and the mixture was stirred under N₂ at room temperature for 16 h. TLC showed the disappearance of the starting material (R_{f} 0,26) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 145 mg (79%) of G152 as a light yellow powder, mp 148-149° C.; ¹H NMR (DMSO-d₆): δ 1.82-2.12 (m, 4H, β-CH₂, CH₂), 2.33-2.37 (t, J=7.6 Hz, 2H, γ-CH₂), 2.64-2.70 (m, 4H, CH₂, CH₂), 4.32-4.37 (m, 1H, α-CH), 6.01 (s, 2H, 2-NH₂, exch), 6.45 (s, 2H, 4-NH₂, exch), 7.17 (s, 1H, C6-CH), 7.43 (d, J=1.2 Hz, 1H, Ar), 7.77 (d, J=1.2 Hz, 1H, Ar), 8.54-8.56 (d, J=7.6 Hz, 1H, CONH, exch), 12.44 (br, 2H, COOH, exch). Anal. calcd for (C₁₉H₂₁N₅O₆S.1.2HCOOH): C, 48.26; H, 4.69; N, 13.93; S, 6.39; Found: C, 48.33; H, 4.83; N, 13.55; S, 6.82. HRMS calcd for C₁₉H₂₁N₅O₆S (M+H)⁺, 448.1285; found: 448.1280.

(S)-2-({5-[3-(2,4-Diamino-furo[2,3-d]pyrimidin-5-yl)-propyl]-thiophene-3-carbonyl}-amino)-pentanedioic acid (G155): To a solution of 431 (200 mg, 0.41 mmol) in MeOH (10 mL) was added 1 N NaOH (10 mL) and the mixture was stirred under N₂ at room temperature for 16 h. TLC showed the disappearance of the starting material (R_{f} 026) and one major spot at the origin (CHCl₃/MeOH 10:1). The reaction mixture was evaporated to dryness under reduced pressure. The residue was dissolved in water (10 mL), the resulting solution was cooled in an ice bath, and the pH was adjusted to 3-4 with dropwise addition of 1 N HCl. The resulting suspension was frozen in a dry ice-acetone bath, thawed to 4-5° C. in the refrigerator, and filtered. The residue was washed with a small amount of cold water and dried in vacuum using P₂O₅ to afford 149 mg (81%) of G155 as a light yellow powder. mp 148-149° C.; ¹H NMR (DMSO-d₆): δ 1.82-2.11 (m, 4H, β-CH₂, CH₂), 2.32-2.36 (t, J=7.6 Hz, 2H, γ-CH₂), 2.68-2.71 (t, J=7.6 Hz, 2H, CH₂), 2.84-2.88 (t, J=7.6 Hz, 2H, CH₂), 4.32-4.38 (m, 1H, α-CH), 5.99 (s, 2H, 2-NH₂, exch), 6.44 (s, 2H, 4-NH₂, exch), 7.16 (s, 1H, C6-CH), 7.30 (d, J=1.2 Hz, 1H, Ar), 7.98 (d, J=1.2 Hz, 1H, Ar), 8.34-8.36 (d, J=7.6 Hz, 1H, CONH, exch), 12.42 (br, 2H, COOH, exch). Anal. calcd for (C₁₉H₂₁N₅O₆S.0.3CHCl₃): C, 47.97; H, 4.44; N, 14.49; S, 6.64; Found: C, 48.36; H, 4.58; N, 14.25; S, 6.45.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound comprising Formula I: 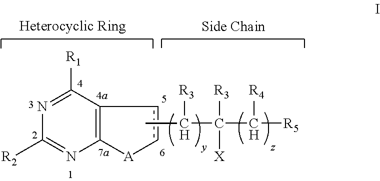
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7; and
z is an integer ranging from zero up to and including seven, wherein the sum total of integers y and z is equal to or less than seven.

2. The compound of claim 1 comprising wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

3. The compound of claim 1 comprising wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

4. The compound of claim 1 comprising wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

5. The compound of claim 1 comprising wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

6. The compound of claim 1 comprising wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

7. The compound of claim 1 comprising said Side Chain having one or more carbon to carbon double or triple bonds between the carbon atoms of (C)_{y} and (C)_{z}.

8. The compound of claim 1 comprising wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

9. The compound of claim 1 comprising wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

10. The compound of claim 1 wherein said Side Chain of Formula I comprises zero or one or more double bonds comprising E-isomers and Z-isomers.

11. The compound of claim 1 comprising pharmaceutically acceptable salts, prodrugs, solvates or hydrates thereof.

12. A pharmaceutical composition comprising a therapeutically effective amount of a compound comprising Formula I: 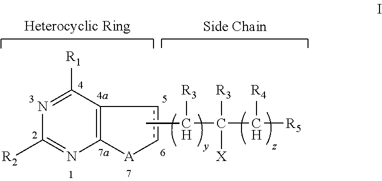
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A is one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7; and
z is an integer ranging from zero up to and including seven, wherein the sum total of integers y and z is equal to or less than seven.

13. The pharmaceutical composition of claim 12 comprising wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

14. The pharmaceutical composition of claim 12 comprising wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

15. The pharmaceutical composition of claim 12 comprising wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

16. The pharmaceutical composition of claim 12 comprising wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

17. The pharmaceutical composition of claim 12 comprising wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof. group consisting of a dihydrothiophene-L-glutamate group, a tetrahydrothiophene-L-glutamate group, a dihydrofuran-L-glutamate group, a tetrahydrofuran-L-glutamate group, a dihydropyrrole-L-glutamate group, and a tetrahydropyrrole-L-glutamate group.

18. The pharmaceutical composition of claim 12 comprising said Side Chain having one or more carbon to carbon double or triple bonds between the carbon atoms of (C)_{y} and (C)_{z}.

19. The pharmaceutical composition of claim 12 comprising wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

20. The pharmaceutical composition of claim 12 comprising wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

21. The pharmaceutical composition of claim 12 wherein said Side Chain of Formula I comprises zero or one or more double bonds comprising E-isomers and Z-isomers.

22. The pharmaceutical composition of claim 12 comprising pharmaceutically acceptable salts, prodrugs, solvates or hydrates thereof.

23. A method of treating a patient diagnosed with cancer comprising administering to the patient a therapeutically effective amount of a compound of Formula 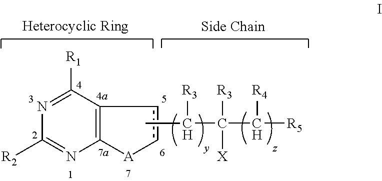
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A comprises one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7; and
z is an integer ranging from zero up to and including seven, wherein the sum total of integers y and z is equal to or less than seven.

24. The method of claim 23 including comprising said compound wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

25. The method of claim 23 including comprising said compound wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

26. The method of claim 23 including comprising said compound wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

27. The method of claim 23 including comprising said compound wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

28. The method of claim 23 including comprising wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

29. The method of claim 23 including comprising said compound wherein said Side Chain having one or more carbon to carbon double or triple bonds between the carbon atoms of (C)_{y} and (C)_{z}.

30. The method of claim 23 including comprising said compound wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

31. The method of claim 30 including comprising said compound wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

32. The method of claim 23 including comprising said compound wherein said Side Chain of Formula I comprises zero or one or more double bonds comprising E-isomers and Z-isomers.

33. The method of claim 23 including comprising pharmaceutically acceptable salts, prodrugs, solvates or hydrates of said compound.

34. A method for targeting cancerous cells via the proton coupled folate transporter pathway comprising:
(a) providing a compound comprising Formula I: 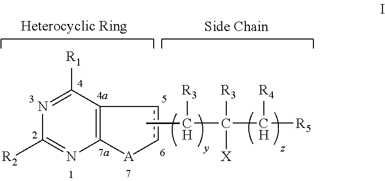
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A comprises one of (a) CR′, and
(b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7;
z is an integer ranging from zero up to and including seven, wherein the sum total of integers y and z is equal to or less than seven;
(b) subjecting cancerous cells expressing a human proton coupled folate transporter (PCFT) to said compound of Formula I;
(c) establishing selective binding of said compound of Formula Ito said human PCFT; and
(d) effecting the selective transport of said compound of Formula I bound to said human PCFT to a target cancerous cell wherein said compound of Formula I acts as a growth inhibitor of said target cancerous cells and inhibits GARFTase within said target cancerous cells.

35. The method of claim 34 including wherein said compound of Formula I is selective for receptors of FR alpha and human PCFT associated with expressing cancerous cells.

36. The method of claim 34 including wherein said Compound of Formula I is not significantly taken up by tissues or cells using the reduced folate carrier system.

37. The method of claim 34 including comprising said compound wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

38. The method of claim 34 including comprising said compound wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

39. The method of claim 34 including comprising the compound wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

40. The method of claim 34 including comprising said compound wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

41. The method of claim 34 including comprising said compound wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

42. The method of claim 34 comprising including said compound wherein said Side Chain has one or more carbon to carbon double or triple bonds between the carbon atoms of (C)_{y} and (C)_{z}.

43. The method of claim 34 including comprising said compound wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

44. The method of claim 43 including comprising wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

45. The method of claim 34 including comprising said compound wherein said Side Chain of Formula I comprises zero or one or more double bonds comprising E-isomers and Z-isomers.

46. The method of claim 34 including comprising a pharmaceutically acceptable salt, prodrug, solvate or hydrate of said compound.

47. The method of claim 34 including wherein said compound targets cancerous cells selected from the group consisting of ovarian, breast, cervical, kidney, and brain tumors.

48. A method for inhibiting GARFTase in cancerous cells comprising:
(a) providing a compound of Formula I having a cytotoxic affect: 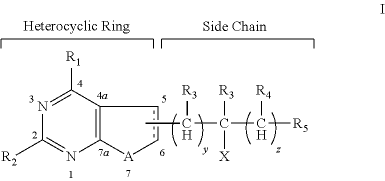
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A comprises one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7;
z is an integer ranging from zero up to and including seven, wherein the sum total of integers y and z is equal to or less than seven;
(b) selectively delivering said compound to said cancerous cell;
(c) effecting the entry of said compound into said cancerous cell;
(d) retaining said compound in said cancerous cell for a sufficient amount of time for effecting binding of said compound with a GARFTase enzyme; and
(e) lysing of said cancerous cell via said binding of said compound with said GARFTase enzyme and inhibiting the DNA replication of said cancerous cell.

49. The method of claim 48 including comprising wherein said compound is selective for receptors of FR alpha associated with expressing cancerous cells.

50. The method of claim 48 including comprising said compound wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

51. The method of claim 48 including comprising said compound wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

52. The method of claim 48 including comprising said compound wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

53. The method of claim 48 including comprising said compound wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

54. The method of claim 48 including comprising said compound wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

55. The method of claim 48 including comprising said compound wherein said Side Chain has one or more carbon to carbon double or triple bonds between the carbon atoms of (C)_{y} and (C)_{z}.

56. The method of claim 48 including comprising said compound wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

57. The method of claim 56 including comprising said compound wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

58. The method of claim 48 including comprising said compound wherein said Side Chain of Formula I comprises zero or one or more double bonds comprising E-isomers and Z-isomers.

59. The method of claim 48 including comprising a pharmaceutically acceptable salt, prodrug, solvate or hydrate of said compound.

60. A method for selectively targeting activated macrophages in a patient having an autoimmune disease comprising:
(a) providing a compound comprising Formula I: 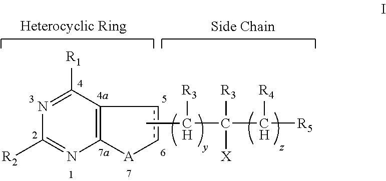
wherein R₁ is one of (a) a hydrogen (H)), (b) an OH, (c) a CH₃, and (d) NHR wherein R is either a H or an alkyl group having from 1 to 6 carbon atoms, and tautomers of said (b) and said (d);
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 may either be a single or a double bond;
wherein the five membered ring has a Side Chain attached at positions 5, 6 or 7, and wherein when said Side Chain attachment is at position 7 then A comprises one of (a) CR′, and (b) N, and optionally includes wherein the carbon atoms at positions 5 and 6, independently, have attached thereto either (a) two hydrogen atoms if the bond between carbon atoms 5 and 6 is a single bond or one hydrogen atom if the bond between carbon atoms 5 and 6 is a double bond, or (b) an alkyl group having from one to six carbon atoms and a hydrogen atom if the bond between carbon atoms at positions 5 and 6 is a single bond or an alkyl group having from one to six carbon atoms if the bond between carbon atoms 5 and 6 is a double bond, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
X is either a heterocycloalkyl-carbonyl-L-glutamate group, a heterocycloaryl-carbonyl-L-glutamate group, or a hydrogen (H), and wherein X is a hydrogen then R₄ is a heterocycloalkyl-carbonyl-L-glutamate group or a heterocycloaryl-carbonyl-L-glutamate group, and wherein X is a heterocycloalkyl-carbonyl-L-glutamate group or a heterocycloaryl-carbonyl-L-glutamate group then R₄ is a hydrogen or a bond;
wherein R₅ is the same as R₃ except that R₅ is not a bond;
y is an integer ranging from zero up to and including 7;
z is an integer ranging from zero up to and including seven, wherein the sum total of integers y and z is equal to or less than seven;
(b) subjecting an activated macrophage expressing a folate receptor (FR) to said compound of Formula I;
(c) establishing selective binding of said compound of Formula I to said FR; and
(d) effecting the selective transport of said compound of Formula I bound to said FR to a target activated macrophage of the autoimmune disease wherein said compound of Formula I acts as an inhibitor of said activated macrophage's release of destructive inflammatory mediators.

61. The method of claim 60 including wherein said compound of Formula I is selective for receptors of FR alpha and human proton coupled folate transporter associated with expressing macrophage cells.

62. The method of claim 60 including wherein the autoimmune disease having said activated macrophage cell expressing said FR is rheumatoid arthritis.

63. The method of claim 60 including comprising said compound wherein said Side Chain attachment is at carbon atom position 6 and wherein A is said CR′R″, and further comprising wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

64. The method of claim 60 including comprising said compound wherein said Side Chain attachment is at carbon atom position 6 and wherein A is NR′ wherein R′ is either a hydrogen atom or an alkyl group having from one to six carbon atoms, and wherein the carbon atom at position 5, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

65. The method of claim 60 including comprising the compound wherein said Side Chain attachment is at carbon atom position 5 and wherein and wherein A is said CR′R″, and further comprising wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

66. The method of claim 60 including comprising said compound wherein said Side Chain attachment is at carbon atom position 5 and wherein A is NR′ wherein R′ is either a hydrogen atom or an alkyl group having from one to six carbon atoms, and wherein the carbon atom at position 6, independently has attached thereto either (a) two hydrogen atoms if the bond between carbon atoms at positions 5 and 6 is a single bond or one hydrogen atom if the bond between carbon atoms at positions 5 and 6 is a double bond, or (b) an alkyl group having from one to six carbon atoms if the bond between carbon atoms of positions 5 and 6 is a double bond or an alkyl group having from one to six carbon atoms and a hydrogen atom if the bond between carbon atoms at positions 5 and 6 is a single bond, and combinations thereof.

67. The method of claim 60 including comprising said compound wherein said heterocycloalkyl-carbonyl-L-glutamate group is selected from the group consisting of a dihydrothiophene-carbonyl-L-glutamate group, a tetrahydrothiophene-carbonyl-L-glutamate group, a dihydrofuran-carbonyl-L-glutamate group, a tetrahydrofuran-carbonyl-L-glutamate group, a dihydropyrrole-carbonyl-L-glutamate group, a tetrahydropyrrole-carbonyl-L-glutamate group, a monohydropyridyl-carbonyl-L-glutamate group, a dihydropyridyl-carbonyl-L-glutamate group, and a piperidyl-carbonyl-L-glutamate group, and stereoisomers thereof.

68. The method of claim 60 comprising including said compound wherein said Side Chain has one or more carbon to carbon double or triple bonds between the carbon atoms of (C)_{y} and (C)_{z}.

69. The method of claim 60 including comprising said compound wherein A is NR′ and R′ is a hydrogen atom, and wherein y is from one to six carbon atoms, z is zero, R₃, and R₅ are each hydrogen atoms, and X is selected from the group consisting of a heterocycloalkyl-carbonyl-L-glutamate group and a heterocycloaryl-carbonyl-L-glutamate group.

70. The method of claim 60 including comprising wherein said heterocycloaryl-carbonyl-L-glutamate group is selected from the group consisting of a thiophene-carbonyl-L-glutamate group, a furan-carbonyl-L-glutamate group, a pyrrole-carbonyl-L-glutamate group, and a pyridine-carbonyl-L-glutamate group.

71. The method of claim 60 including comprising said compound wherein said Side Chain of Formula I comprises zero or one or more double bonds comprising E-isomers and Z-isomers.

72. The method of claim 60 including comprising a pharmaceutically acceptable salt, prodrug, solvate or hydrate of said compound of Formula I.

73. The method of claim 60 including delivering said compound of Formula I or a pharmaceutically acceptable salt, prodrug, solvate or hydrate of said compound of Formula I by injection into a joint or synovial fluid of a patient.

74. A compound comprising Formula 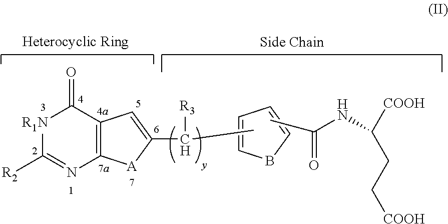
wherein R₁ is one of a hydrogen (H) or an alkyl group having from 1 to 6 carbon atoms;
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 is a double bond;
wherein the five membered ring of the Heterocyclic Ring of Formula II has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
B is one of (a) a sulfur (S) atom, (b) an oxygen (O) atom, (c) CH₂, or (d) a NR′; and
y is an integer ranging from zero up to and including 8; and
wherein the (CH)_{y} of said Side Chain of Formula II is attached to the five membered ring of said Side Chain of Formula II at any one of positions 2, 3, 4, and 5 of said five membered ring of said Side Chain of Formula II (numbering clockwise from element B as position 1 of said five membered ring of said Side Chain of Formula II), and wherein the carbonyl-L-glutamate substituent of the Side Chain of Formula II is attached to the five membered ring of said Side Chain of Formula II at any one of said positions 2, 3, 4, and 5, except that said (CH)_{y} and said carbonyl-L-glutamate are attached at different positions of said five membered ring of said Side Chain of Formula II.

75. The compound of claim 74 comprising said Side Chain having one or more carbon to carbon double or triple bonds between the carbon atoms of (C)_{y 1-8}.

76. The compound of claim 74 wherein said Side Chain of Formula II comprises zero or one or more double bonds comprising E-isomers and Z-isomers.

77. The compound of claim 74 comprising one of a pharmaceutically acceptable salt, prodrug, solvate, or hydrate thereof.

78. The compound of claim 74 including tautomers of said Heterocyclic Ring of Formula II.

79. The compound of claim 78 wherein said tautomers are of a keto-enol form, or a lactam-lactim form.

80. The compound of claim 74 including positional regioisomers, geometric isomers, optical isomers, and conformational isomers of said Formula II.

81. A pharmaceutical composition comprising a therapeutically effective amount of a compound of claim 74.

82. The method of claim 48 including inhibiting AICARFTase by providing the compound of Formula I wherein A is a sulfur atom.

83. A compound comprising Formula III: 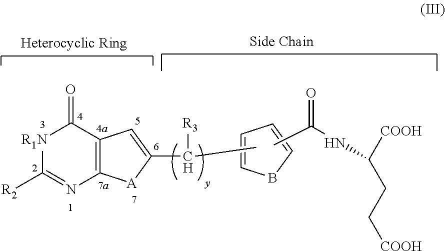
wherein R₁ is one of a hydrogen (H) or an alkyl group having from 1 to 6 carbon atoms;
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR, wherein R′ and R″ are the same or different and are either a H or an alkyl group having from 1 to 6 carbon atoms, (c) a sulfur (S), and (d) an oxygen (O);
wherein the bond at position 5-6 is a double bond;
wherein the five membered ring of the Heterocyclic Ring of Formula III has a Side Chain attached at position 6, and optionally includes wherein the carbon atoms at positions 5 and 6, independently, have attached thereto either (a) one hydrogen atom, or (b) an alkyl group having from one to six carbon atoms, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
B is one of (a) a sulfur (S) atom, (b) an oxygen (O) atom, (c) CH₂, or (d) a NR′; and
y is an integer ranging from zero up to and including 8; and
wherein the (CH)_{y} of said Side Chain of Formula III is attached to the five membered ring of said Side Chain of Formula III at any one of positions 2, 3, 4, and 5 of said five membered ring of said Side Chain of Formula III (numbering clockwise from element B as position 1 of said five membered ring of said Side Chain of Formula III), and wherein the carbonyl-L-glutamate substituent of the Side Chain of Formula III is attached to the five membered ring of said Side Chain of Formula III at any one of said positions 2, 3, 4, and 5, except that said (CH)_{y} and said carbonyl-L-glutamate are attached at different positions of said five membered ring of said Side Chain of Formula III.

84. The compound of claim 83 comprising said Side Chain having one or more carbon to carbon double or triple bonds between the carbon atoms of (C)_{y 1-8}.

85. The compound of claim 83 wherein said Side Chain of Formula III comprises zero or one or more double bonds comprising E-isomers and Z-isomers.

86. The compound of claim 83 comprising one of a pharmaceutically acceptable salt, prodrug, solvate, or hydrate thereof.

87. The compound of claim 83 including tautomers of said Heterocyclic Ring of Formula III.

88. The compound of claim 87 wherein said tautomers are of a keto-enol form, or a lactam-lactim form.

89. The compound of claim 83 including positional regioisomers, geometric isomers, optical isomers, and conformational isomers of said Formula III

90. A pharmaceutical composition comprising a therapeutically effective amount of a compound of claim 83.

91. A compound comprising Formula IV: 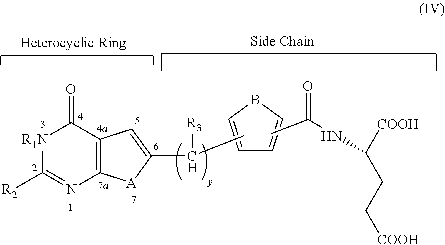
wherein R₁ is one of a hydrogen (H) or an alkyl group having from 1 to 6 carbon atoms;
R₂ is one of (a) a hydrogen (H), (b) a CH₃, (c) an OH, and (d) NHR wherein R is either a H or an alkyl group having from 1 to 6 carbon atoms;
A is one of (a) CR′R″, (b) NR′, wherein R′ and R″ are the same or different and are either a H or an alkyl group having from 1 to 6 carbon atoms, (c) a sulfur (S), and (d) an oxygen (O);
wherein the bond at position 5-6 is a double bond;
wherein the five membered ring of the Heterocyclic Ring of Formula IV has a side chain attached at position 6, and optionally includes wherein the carbon atoms at positions 5 and 6, independently, have attached thereto either (a) one hydrogen atom, or (b) an alkyl group having from one to six carbon atoms, and combinations thereof, and
R₃ is one of (a) a hydrogen (H), (b) CH₃, (c) trifluoromethyl, (d) difluoromethyl, (e) monofluoromethyl, (f) methyl ketone, (g) trifluoromethyl ketone, (h) difluoromethyl ketone, (i) monofluoromethyl ketone, (j) formyl, (k) methyl alcohol, (l) methylamine, or (m) a bond;
B is one of (a) a sulfur (S) atom, (b) an oxygen (O) atom, (c) CH₂, or (d) a NR′; and
y is an integer ranging from zero up to and including 8; and
wherein the (CH)_{y} of said Side Chain of Formula IV is attached to the five membered ring of said Side Chain of Formula IV at any one of positions 2, 3, 4, and 5 of said five membered ring of said Side Chain of Formula IV (numbering clockwise from element B as position 1 of said five membered ring of said Side Chain of Formula IV), and wherein the carbonyl-L-glutamate substituent of the Side Chain of Formula IV is attached to the five membered ring of said Side Chain of Formula IV at any one of said positions 2, 3, 4, and 5, except that said (CH)_{y} and said carbonyl-L-glutamate are attached at different positions of said five membered ring of said Side Chain of Formula IV.

92. The compound of claim 91 comprising said side chain having one or more carbon to carbon double or triple bonds between the carbon atoms of (C)_{y 1-8}.

93. The compound of claim 91 wherein said side chain of Formula IV comprises zero or one or more double bonds comprising E-isomers and Z-isomers.

94. The compound of claim 91 comprising one of a pharmaceutically acceptable salt, prodrug, solvate, or hydrate thereof.

95. The compound of claim 91 including tautomers of said Heterocyclic Ring of Formula IV.

96. The compound of claim 95 wherein said tautomers are of a keto-enol form, or a lactam-lactim form.

97. The compound of claim 91 including positional regioisomers, geometric isomers, optical isomers, and conformational isomers of said Formula IV.

98. A pharmaceutical composition comprising a therapeutically effective amount of a compound of claim 91.

99. A compound comprising Formula V: 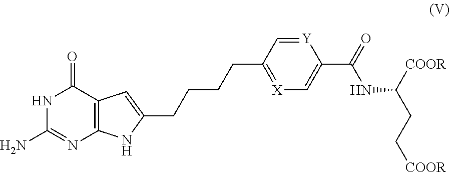
wherein X is N or CH;
Y is N when X is CH or Y is CH when X is N; and
R is H.

100. The compound of claim 99 comprising one of a pharmaceutically acceptable salt, prodrug, solvate, or hydrate thereof.

101. The compound of claim 99 including tautomers of said heterocyclic ring of Formula V.

102. The compound of claim 101 wherein said tautomers are of a keto-enol form, or a lactam-lactim form.

103. The compound of claim 99 including positional regioisomers, geometric isomers, optical isomers, and conformational isomers of said Formula V.

104. A pharmaceutical composition comprising a therapeutically effective amount of a compound of claim 99.

105. A compound comprising Formula VI: 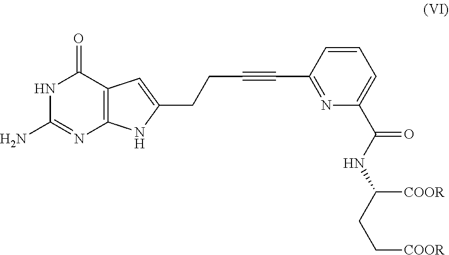
wherein R is H.

106. The compound of claim 105 comprising one of a pharmaceutically acceptable salt, prodrug, solvate, or hydrate thereof.

107. The compound of claim 105 including tautomers of the heterocyclic ring of Formula VI.

108. The compound of claim 107 wherein said tautomers are of a keto-enol form, or a lactam-lactim form.

109. The compound of claim 105 including positional regioisomers, geometric isomers, optical isomers, and conformational isomers of said Formula VI.

110. A pharmaceutical composition comprising a therapeutically effective amount of a compound of claim 105.

111. A compound comprising Formula VII: 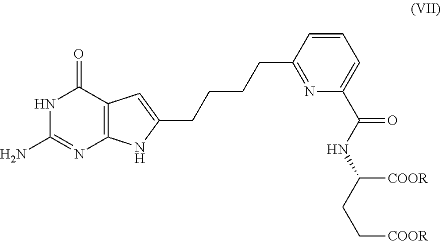
wherein R is H.

112. The compound of claim 111 comprising one of a pharmaceutically acceptable salt, prodrug, solvate, or hydrate thereof.

113. The compound of claim 111 including tautomers of said heterocyclic ring of Formula VII.

114. The compound of claim 113 wherein said tautomers are of a keto-enol form, or a lactam-lactim form.

115. The compound of claim 111 including positional regioisomers, geometric isomers, optical isomers, and conformational isomers of said Formula VII.

116. A pharmaceutical composition comprising a therapeutically effective amount of a compound of claim 111.

117. A compound comprising Formula VIII: 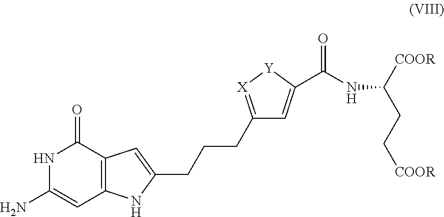
wherein X is CH or S;
Y is S when X is CH and Y is CH when X is S; and
R is H.

118. The compound of claim 117 comprising one of a pharmaceutically acceptable salt, prodrug, solvate, or hydrate thereof.

119. The compound of claim 117 including tautomers of the heterocyclic ring of Formula VIII.

120. The compound of claim 119 wherein said tautomers are of a keto-enol form, or a lactam-lactim form.

121. The compound of claim 117 including positional regioisomers, geometric isomers, optical isomers, and conformational isomers of said Formula VIII.

122. A pharmaceutical composition comprising a therapeutically effective amount of a compound of claim 117.

123. A compound comprising Formula IX: 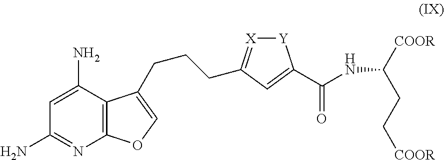
wherein X is CH or S;
Y is S when X is CH and Y is CH when X is S; and
R is H.

124. The compound of claim 123 comprising one of a pharmaceutically acceptable salt, prodrug, solvate, or hydrate thereof.

125. The compound of claim 123 including tautomers of the heterocyclic ring of Formula IX.

126. The compound of claim 125 wherein said tautomers are of a keto-enol form, or a lactam-lactim form.

127. The compound of claim 123 including positional regioisomers, geometric isomers, optical isomers, and conformational isomers of said Formula IX.

128. A pharmaceutical composition comprising a therapeutically effective amount of a compound of claim 123.

129. A compound comprising the Formula X: 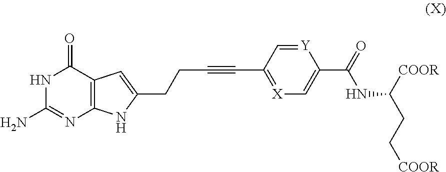
wherein X is CH or N; wherein Y is CH when X is N and Y is N when X is CH; and wherein R is H.

130. The compound of claim 129 comprising one of a pharmaceutically acceptable salt, prodrug, solvate, or hydrate thereof.

131. The compound of claim 129 including tautomers of the heterocyclic ring of Formula X.

132. The compound of claim 129 wherein said tautomers are of a keto-enol form, or a lactam-lactim form.

133. The compound of claim 129 including positional regioisomers, geometric isomers, optical isomers, and conformational isomers of said Formula X.

134. A pharmaceutical composition comprising a therapeutically effective amount of a compound of claim 129.
